(19) United States
(12) Patent Application Publication
(10) Pub. No.: US 2013/0143235 A1
(43) Pub. Date: Jun. 6, 2013

(54) COMPARATIVE LIGAND MAPPING FROM MHC CLASS I POSITIVE CELLS

(71) Applicant: THE BOARD OF REGENTS OF THE UNIVERSIT, Norman, OK (US)

(72) Inventors: William H. Hildebrand, Edmond, OK (US); Oriana Hawkins, Shawnee, OK (US)

(73) Assignee: THE BOARD OF REGENTS OF THE UNIVERSITY OF OKLAHOMA, Norman, OK (US)

(21) Appl. No.: 13/705,957

(22) Filed: Dec. 5, 2012

Related U.S. Application Data
(63) Continuation of application No. 13/186,567, filed on Jul. 20, 2011, now abandoned.
Application No. 13/186,567 is a division of application No. 12/214,348, filed on Jun. 18, 2008, now abandoned.
Application No. 12/214,348 is a continuation-in-part of application No. 11/591,118, filed on Nov. 1, 2006, now abandoned.
Application No. 11/591,118 is a continuation-in-part of application No. 10/845,391, filed on May 13, 2004, now abandoned.
Application No. 10/845,391 is a continuation-in-part of application No. 09/974,366, filed on Oct. 10, 2001, now Pat. No. 7,541,429.
(60) Provisional application No. 60/936,050, filed on Jun. 18, 2007.
(60) Provisional application No. 60/732,183, filed on Nov. 1, 2005.
(60) Provisional application No. 60/800,134, filed on May 12, 2006.
(60) Provisional application No. 60/469,995, filed on May 13, 2003.
(60) Provisional application No. 60/518,132, filed on Nov. 7, 2003.
(60) Provisional application No. 60/240,143, filed on Oct. 10, 2000.
(60) Provisional application No. 60/299,452, filed on Jun. 20, 2001.
(60) Provisional application No. 60/256,410, filed on Dec. 18, 2000.
(60) Provisional application No. 60/256,409, filed on Dec. 18, 2000.
(60) Provisional application No. 60/327,907, filed on Oct. 9, 2001.

(51) Int. Cl. G01N 33/68

(52) U.S. Cl. CPC G01N 33/6878
USPC 435/7.21; 530/394

(57) ABSTRACT

Compositions that include isolated, functionally active, recombinantly produced class I HLA trimolecular complexes that include epitopes unique to breast cancer cells are disclosed.

### INCORPORATION BY REFERENCE

The entire contents of the below referenced patents and patent applications are hereby expressly incorporated herein by reference: U.S. Ser. No. 13/186,567, filed Jul. 20, 2011; U.S. Ser. No. 12/214,348, filed Jun. 18, 2008; U.S. Ser. No. 60/936,050, filed Jun. 18, 2007; U.S. Ser. No. 11/591,118, filed Nov. 1, 2006; U.S. Ser. No. 60/732,183, filed Nov. 1, 2005; U.S. Ser. No. 60/800,134, filed May 12, 2006; U.S. Ser. No. 10/845,391, filed May 13, 2004; U.S. Ser. No. 60/469,995, filed May 13, 2003; U.S. Ser. No. 60/518,132, filed Nov. 7, 2003; U.S. Ser. No. 09/974,366, filed Oct. 10, 2001; U.S. Ser. No. 60/240,143, filed Oct. 10, 2000; U.S. Ser. No. 60/299,452, filed Jun. 20, 2001; U.S. Ser. No. 60/256,410, filed Dec. 18, 2000; U.S. Ser. No. 60/256,409, filed Dec. 18, 2000; and U.S. Ser. No. 60/327,907, filed Oct. 9, 2001.

### STATEMENT REGARDING FEDERALLY SPONSORED RESEARCH OR DEVELOPMENT

This invention was made with government support under NIST Grant No. 70NANB4H3048 awarded by the Advanced Technology Program and Grant Research Fellowship No. 2006036207 from the National Science Foundation. The government has certain rights in this invention.

### BACKGROUND OF THE INVENTION

1. Field of the Invention

The presently disclosed and claimed inventive concept(s) relates generally to a methodology of epitope testing for the identification of peptides that bind to an individual soluble MHC Class I or Class II molecule as well as to peptides identified by such methodology.

2. Description of the Background Art

Class I major histocompatibility complex (MHC) molecules, designated HLA class I in humans, bind and display peptide antigen ligands upon the cell surface. The peptide antigen ligands presented by the class I MHC molecule are derived from either normal endogenous proteins (“self”) or foreign proteins (“non-self”) introduced into the cell. Non-self proteins may be products of malignant transformation or intracellular pathogens such as viruses. In this manner, class I MHC molecules convey information regarding the internal fitness of a cell to immune effector cells including but not limited to, CD8⁺ cytotoxic T lymphocytes (CTLs), which are activated upon interaction with “non-self” peptides, thereby lysing or killing the cell presenting such “non-self” peptides.

Class II MHC molecules, designated HLA class II in humans, also bind and display peptide antigen ligands upon the cell surface. Unlike class I MHC molecules which are expressed on virtually all nucleated cells, class II MHC molecules are normally confined to specialized cells, such as B lymphocytes, macrophages, dendritic cells, and other antigen presenting cells which take up foreign antigens from the extracellular fluid via an endocytic pathway. The peptides they bind and present are derived from extracellular foreign antigens, such as products of bacteria that multiply outside of cells, wherein such products include protein toxins secreted by the bacteria that often times have deleterious and even lethal effects on the host (e.g. human). In this manner, class II molecules convey information regarding the fitness of the extracellular space in the vicinity of the cell displaying the class II molecule to immune effector cells, including but not limited to, CD4⁺ helper T cells, thereby helping to eliminate such pathogens the examination of such pathogens is accomplished by both helping B cells make antibodies against microbes, as well as toxins produced by such microbes, and by activating macrophages to destroy ingested microbes.

Class I and class II HLA molecules exhibit extensive polymorphism generated by systematic recombinatorial and point mutation events; as such, hundreds of different HLA types exist throughout the world's population, resulting in a large immunological diversity. Such extensive HLA diversity throughout the population results in tissue or organ transplant rejection between individuals as well as differing susceptibilities and/or resistances to infectious diseases. HLA molecules also contribute significantly to autoimmunity and cancer. Because HLA molecules mediate most, if not all, adaptive immune responses, large quantities of pure isolated HLA proteins are required in order to effectively study transplantation, autoimmunity disorders, and for vaccine development.

There are several applications in which purified, individual class I and class II MHC proteins are highly useful. Such applications include using MHC-peptide multimers as immunodiagnostic reagents for disease resistance/autoimmunity; assessing the binding of potentially therapeutic peptides; elution of peptides from MHC molecules to identify vaccine candidates; screening transplant patients for preformed MHC specific antibodies; and removal of anti-HLA antibodies from a patient. Since every individual has differing MHC molecules, the testing of numerous individual MHC molecules is a prerequisite for understanding the differences in disease susceptibility between individuals. Therefore, purified MHC molecules representative of the hundreds of different HLA types existing throughout the world's population are highly desirable for unraveling disease susceptibilities and resistances, as well as for designing therapeutics such as vaccines.

Class I HLA molecules alert the immune response to disorders within host cells. Peptides, which are derived from viral- and tumor-specific proteins within the cell, are loaded into the class I molecule's antigen binding groove in the endoplasmic reticulum of the cell and subsequently carried to the cell surface. Once the class I HLA molecule and its loaded peptide ligand are on the cell surface, the class I molecule and its peptide ligand are accessible to cytotoxic T lymphocytes (CTL). CTL survey the peptides presented by the class I molecule and destroy those cells harboring ligands derived from infectious or neoplastic agents within that cell.

While specific CTL targets have been identified, little is known about the breadth and nature of ligands presented on the surface of a diseased cell. From a basic science perspective, many outstanding questions have percolated through the art regarding peptide exhibition. For instance, it has been demonstrated that a virus can preferentially block expression of HLA class I molecules from a given locus while leaving expression at other loci intact. Similarly, there are numerous reports of cancerous cells that fail to express class I HLA at particular loci. However, there is no data describing how (or if) the three classical HLA class I loci differ in the immunoregulatory ligands they bind. It is therefore unclear how class I molecules from the different loci vary in their interaction with viral- and tumor-derived ligands and the number of peptides each will present.

Discerning virus- and tumor-specific ligands for CTL recognition is an important component of vaccine design. Ligands unique to tumorigenic or infected cells can be tested and incorporated into vaccines designed to evoke a protective CTL response. Several methodologies are currently employed to identify potentially protective peptide ligands. One approach uses T cell lines or clones to screen for biologically active ligands among chromatographic fractions of eluted peptides (Cox et al., Science, vol. 264, 1994, pages 716-719, which is expressly incorporated herein by reference in its entirety). This approach has been employed to identify peptide ligands specific to cancerous cells. A second technique utilizes predictive algorithms to identify peptides capable of binding to a particular class I molecule based upon previously determined motif and/or individual ligand sequences (DeGroot et al., Emerging Infectious Diseases, (7) 4, 2001, which is expressly incorporated herein by reference in its entirety). Peptides having high predicted probability of binding from a pathogen of interest can then be synthesized and tested for T cell reactivity in various assays, such as but not limited to, precursor, tetramer and ELISpot assays.

However, there has been no readily available source of individual HLA molecules. The quantities of HLA protein available have been small and typically consist of a mixture of different HLA molecules. Production of HLA molecules traditionally involves growth and lysis of cells expressing multiple HLA molecules. Ninety percent of the population is heterozygous at each of the HLA loci; codominant expression results in multiple HLA proteins expressed at each HLA locus. To purify native class I or class II molecules from mammalian cells requires time-consuming and cumbersome purification methods, and since each cell typically expresses multiple surface-bound HLA class I or class II molecules, HLA purification results in a mixture of many different HLA class I or class II molecules. When performing experiments using such a mixture of HLA molecules or performing experiments using a cell having multiple surface-bound HLA molecules, interpretation of results cannot directly distinguish between the different HLA molecules, and one cannot be certain that any particular HLA molecule is responsible for a given result. Therefore, prior to the presently disclosed and claimed inventive concept(s), a need existed in the art for a method of producing substantial quantities of individual HLA class I or class II molecules so that they can be readily purified and isolated independent of other HLA class I or class II molecules. Such individual HLA molecules, when provided in sufficient quantity and purity as described herein, provides a powerful tool for studying and measuring immune responses.

Therefore, there exists a need in the art for improved methods of assaying binding of peptides to class I and class II MHC molecules to identify epitopes that bind to specific individual class I and class II MHC molecules. The presently disclosed and claimed inventive concept(s) solves this need by coupling the production of soluble HLA molecules with epitope isolation, discovery, and testing methodology.

### BRIEF DESCRIPTION OF THE DRAWINGS

FIG. 1. Overview of 2 stage PCR strategy to amplify a truncated version of the human class I MHC.

FIG. 2. Flow chart of the epitope discovery of C-terminal-tagged sHLA molecules. Class I positive transfectants are infected with a pathogen of choice, and sHLA is preferentially purified utilizing the tag. Subtractive comparison of MS ion maps yields ions present only in infected cell, which are then MS/MS sequenced to derive class I epitopes.

FIG. 3. MS ion map showing a unique +2 peak at 536.32 m/z in corresponding peptide fractions from (A) MCF-7, (B) MDA-MB-231, (C) BT-20, and (D) the non-tumorigenic MCF10A. Note: The ion peak at 532.79 m/z is shared by all four cell lines and corresponds to a peptide derived from RPL5.

FIG. 4. Product-ion spectra of an ESI produced +2 ion, 536.32 m/z, in corresponding peptide fractions from (A) MCF-7, (B) MDA-MB-231, (C) BT-20, (D) the non-tumorigenic MCF10A, and (E) Synthetic. Sequence of A-C and E is peptide 23-31 (ILDQKINEV; SEQ ID NO:317) of ODC1.

FIG. 5. MS ion map showing a unique +2 peak at 539.8 m/z in corresponding peptide fractions from (A) MCF-7, (B) MDA-MB-231, (C) BT-20, and (D) the non-tumorigenic MCF10A. Note: Peak 539.76 m/z in panel C is an isotope of 539.26 m/z.

FIG. 6. Product-ion spectra of an ESI produced +2 ion, 539.8 m/z, in corresponding peptide fractions from (A) MCF-7, (B) MDA-MB-231, (C) BT-20, (D) the non-tumorigenic MCF10A, and (E) Synthetic. Sequence of A, B, and E is peptide 19-27 (FLSELTQQL; SEQ ID NO:319) of MIF.

FIG. 7. Western blot showing 75, 53, 32, 28, and 12 kDa bands representing KNTC2, ODC1, Cdk2, EXOSC6, and MIF, respectively. β-Actin is a loading control. Lanes (1) MDA-MB-231, (2) BT-20, (3) MCF-7, and (4) MCF10A cell lysates.

FIG. 8. Tetramer vs. CD8 staining of PBMC from Subject 6. (A) EBV BMLF1-A*0201 tetramer, (B) Cdk2-A*0201 tetramer, (C) ODC1-A*0201 tetramer, (D) EXOSC6-A*0201 tetramer, (E) KNTC2-A*0201 tetramer, and (F) MIF-A*0201 tetramer.

FIG. 9. IFN-γ ELISPOT. Subject PBMC were stimulated with peptide and IL-2 1 week prior to ELISPOT. A total of 1×10⁵ cells/well was plated with 2 μg of peptide or PHA-P as a positive control.

### DETAILED DESCRIPTION OF THE INVENTIVE CONCEPT(S)

Before explaining at least one embodiment of the presently disclosed and claimed inventive concept(s) in detail by way of exemplary drawings, experimentation, results, and laboratory procedures, it is to be understood that the presently disclosed and claimed inventive concept(s) is not limited in its application to the details of construction and the arrangement of the components set forth in the following description or illustrated in the drawings, experimentation and/or results. The presently disclosed and claimed inventive concept(s) is capable of other embodiments or of being practiced or carried out in various ways. As such, the language used herein is intended to be given the broadest possible scope and meaning; and the embodiments are meant to be exemplary—not exhaustive. Also, it is to be understood that the phraseology and terminology employed herein is for the purpose of description and should not be regarded as limiting.

The presently disclosed and claimed inventive concept(s) combines methodologies for assaying the binding of peptide epitopes to individual, soluble MHC molecules with methodologies for the production of individual, soluble MHC molecules and with a method of epitope discovery and comparative ligand mapping (including methods of distinguishing infected/tumor cells from uninfected/non-tumor cells). The method of production of individual, soluble MHC molecules has previously been described in detail in parent application U.S. Publication No. 2003/0166057, filed Dec. 18, 2001, entitled “METHOD AND APPARATUS FOR THE PRODUCTION OF SOLUBLE MHC ANTIGENS AND USES THEREOF,” the contents of which are hereby expressly incorporated herein in their entirety by reference. The method of epitope discovery and comparative ligand mapping has previously been described in detail in parent application U.S. Publication No. 2002/0197672, filed Oct. 10, 2001, entitled “COMPARATIVE LIGAND MAPPING FROM MHC CLASS I POSITIVE CELLS”, the contents of which have previously been expressly incorporated in their entirety by reference. A brief description of each of these methodologies is included herein below for the purpose of exemplification and should not be considered as limiting.

In addition, the methods of the presently disclosed and claimed inventive concept(s) may be combined with methods of epitope testing as described in U.S. Publication No. 2003/0124613, filed Mar. 11, 2002, entitled “EPITOPE TESTING USING SOLUBLE HLA”, the contents of which are hereby expressly incorporated herein by reference.

To produce the individual soluble class I molecule-endogenous peptide complexes, genomic DNA or cDNA encoding at least one class I molecule is obtained, and an allele encoding an individual class I molecule in the genomic DNA or cDNA is identified. The allele encoding the individual class I molecule is PCR amplified in a locus specific manner such that a PCR product produced therefrom encodes a truncated, soluble form of the individual class I molecule. The PCR product is then cloned into an expression vector, thereby forming a construct that encodes the individual soluble class I molecule, and the construct is transfected into a cell line to provide a cell line containing a construct that encodes an individual soluble class I molecule. The cell line must be able to naturally process proteins into peptide ligands capable of being loaded into antigen binding grooves of class I molecules.

The cell line is then cultured under conditions which allow for expression of the individual soluble class I molecules from the construct, and these conditions also allow for endogenous loading of a peptide ligand into the antigen binding groove of each individual soluble class I molecule prior to secretion of the individual soluble class I molecules from the cell. The secreted individual soluble class I molecules having the endogenously loaded peptide ligands bound thereto are then isolated.

The construct that encodes the individual soluble class I molecule may further encode a tag, such as a HIS tail or a FLAG tail, which is attached to the individual soluble class I molecule and aids in isolating the individual soluble class I molecule.

The peptide of interest may be chosen based on several methods of epitope discovery known in the art. Alternatively, the peptide of interest may be identified by a method for identifying at least one endogenously loaded peptide ligand that distinguishes an infected cell from an uninfected cell. Such method includes providing an uninfected cell line containing a construct that encodes an individual soluble class I molecule, wherein the uninfected cell line is able to naturally process proteins into peptide ligands capable of being loaded into antigen binding grooves of class I molecules. A portion of the uninfected cell line is infected with at least one of a microorganism (such as HIV, HBV or influenza), a gene from a microorganism or a tumor gene, thereby providing an infected cell line, and both the uninfected cell line and the infected cell line are cultured under conditions which allow for expression of individual soluble class I molecules from the construct. The culture conditions also allow for endogenous loading of a peptide ligand in the antigen binding groove of each individual soluble class I molecule prior to secretion of the individual soluble class I molecules from the cell. The secreted individual soluble class I molecules having the endogenously loaded peptide ligands bound thereto are isolated from the uninfected cell line and the infected cell line, and the endogenously loaded peptide ligands are separated from the individual soluble class I molecules from both the uninfected cell line and the infected cell line. The endogenously loaded peptide ligands are then isolated from both the uninfected cell line and the infected cell line, and the two sets of endogenously loaded peptide ligands are compared to identify at least one endogenously loaded peptide ligand presented by the individual soluble class I molecule on the infected cell line that is not presented by the individual soluble class I molecule on the uninfected cell line, or to identify at least one endogenously loaded peptide ligand presented by the individual soluble class I molecule in a substantially greater amount on the infected cell line when compared to the uninfected cell line. In addition, the comparison described herein above may also identify at least one endogenously loaded peptide ligand presented by the individual soluble class I molecule on the uninfected cell line that is not presented by the individual soluble class I molecule on the infected cell line, or that is presented in a substantially greater amount on the uninfected cell line when compared to the infected cell line.

The term “substantially greater amount” as used herein refers to an amount that is detectably greater than another amount; for example, the term “presented in a substantially greater amount” as used herein refers to an at least 1-fold increase in a first amount of presentation when compared to a second amount of presentation. The tables provided herein disclose “Fold Increase” amounts for the peptides identified by the methods of the presently disclosed and claimed inventive concept(s).

Optionally, proteomics may eventually allow for sequencing all epitopes from a diseased cell so that comparative mapping, i.e., comparison of infected cells to healthy cells, would no longer be required. Microarrays and other proteomic data should provide insight as to the healthy cell.

Following identification of the peptide ligand that distinguishes an infected cell from an uninfected cell, a source protein from which the endogenously loaded peptide ligand is obtained can be identified. Such source protein may be encoded by at least one of the microorganism, the gene from a microorganism or the tumor gene with which the cell line was infected to form the infected cell line, or the source protein may be encoded by the uninfected cell line. When the source protein is encoded by the uninfected cell line, such protein may also demonstrate increased expression in a tumor cell line.

The methods described herein above may also be utilized to identify peptide ligands that distinguish a tumor cell from a non-tumor cell. Such methods will be performed exactly as described herein above, except that a non-tumorigenic cell may be transformed to become tumorigenic, and the peptide ligands presented by MHC on the surface of both cell types compared as described herein. Optionally, readily available cancer cell line(s) may be utilized and compared with readily available, immortalized, non-tumorigenic cell line(s) from the same tissue/organ as the cancer cell lines.

Therefore, the presently disclosed and claimed inventive concept(s) is also directed to isolated peptide ligands for an individual class I molecule isolated by the methods described herein. In one embodiment, the isolated peptide ligand has a length of from about 7 to about 13 amino acids and consists essentially of a sequence selected from the group consisting of SEQ ID NOS:1-326. In another embodiment, the isolated peptide ligand has a length of from about 7 to about 13 amino acids and consists essentially of a sequence selected from the group consisting of SEQ ID NOS:99-301. In yet another embodiment, the isolated peptide ligand has a length of from about 7 to about 13 amino acids and consists essentially of a sequence selected from the group consisting of SEQ ID NOS:302-315. In yet another embodiment, the isolated peptide ligand has a length of from about 7 to about 13 amino acids and consists essentially of a sequence selected from the group consisting of SEQ ID NOS:316-326.

The isolated peptide ligand described herein above may be an endogenously loaded peptide ligand presented by an individual class I molecule in a substantially greater amount on an infected/tumorigenic cell when compared to an uninfected/non-tumorigenic cell.

The peptide ligands of the presently disclosed and claimed inventive concept(s) may be isolated by a method that includes providing a cell line containing a construct that encodes an individual soluble class I molecule, wherein the cell line is able to naturally process proteins into peptide ligands capable of being loaded into antigen binding grooves of class I molecules. The cell line is cultured under conditions which allow for expression of the individual soluble class I molecules from the construct, and also allowing for endogenous loading of a peptide ligand into the antigen binding groove of each individual soluble class I molecule prior to secretion of the individual soluble class I molecules from the cell. Secreted individual soluble class I molecules having the endogenously loaded peptide ligands bound thereto are then isolated, and the peptide ligands are then separated from the individual soluble class I molecules.

In another embodiment, the isolated peptide ligands of the presently disclosed and claimed inventive concept(s) may be identified by a method that includes providing an uninfected cell line containing a construct that encodes an individual soluble class I molecule, wherein the cell line is able to naturally process proteins into peptide ligands capable of being loaded into antigen binding grooves of class I molecules. A portion of the uninfected cell line is infected with at least one of a microorganism, a gene from a microorganism or a tumor gene, thereby providing an infected cell line. The uninfected cell line and the infected cell line are cultured under conditions which allow for expression of the individual soluble class I molecules from the construct, and also allow for endogenous loading of a peptide ligand in the antigen binding groove of each individual soluble class I molecule prior to secretion of the individual soluble class I molecules from the cell. The secreted individual soluble class I molecules having the endogenously loaded peptide ligands bound thereto are isolated from both the uninfected cell line and the infected cell line; then, the endogenously loaded peptide ligands are separated from the individual soluble class I molecules from the uninfected cell, and the endogenously loaded peptide ligands are separated from the individual soluble class I molecules from the infected cell. The endogenously loaded peptide ligands from the uninfected cell line and the endogenously loaded peptide ligands from the infected cell line are then isolated and compared. Finally, at least one endogenously loaded peptide ligand presented by the individual soluble class I molecule in a substantially greater amount on the infected cell line when compared to the uninfected cell line is identified.

The uninfected cell line containing the construct that encodes the individual soluble class I molecule may be produced by a method that includes obtaining genomic DNA or cDNA encoding at least one class I molecule and identifying an allele encoding an individual class I molecule in the genomic DNA or cDNA. The allele encoding the individual class I molecule is PCR amplified in a locus specific manner such that a PCR product produced therefrom encodes a truncated, soluble form of the individual class I molecule. The PCR product is cloned into an expression vector to form a construct that encodes the individual soluble class I molecule, and the construct is transfected into an uninfected cell line. The construct may further encode a tag, such as but not limited to, a HIS tail or a FLAG tail, which is attached to the individual soluble class I molecule, and the tag aids in isolating the individual soluble class I molecule. The tag may be encoded by a PCR primer utilized in the PCR step, or the tag may be encoded by the expression vector into which the PCR product is cloned.

The at least one endogenously loaded peptide ligand may be obtained from a protein encoded by at least one of the microorganism, the gene from the microorganism or the tumor gene with which the portion of the uninfected cell line is infected to form the infected cell line. Alternatively, the at least one endogenously loaded peptide ligand may be obtained from a protein encoded by the uninfected cell line.

In another embodiment, the isolated peptide ligands of the presently disclosed and claimed inventive concept(s) may be identified by a method similar to that described above, except that rather than providing a cell line and infecting a portion of the cell line to provide an uninfected cell line, two cell lines may be provided. Such cell lines include an immortal, non-tumorigenic cell line and a cancer cell line, wherein both cell lines contain a construct that encodes an individual soluble class I molecule, and wherein both cell lines are able to naturally process proteins into peptide ligands capable of being loaded into antigen binding grooves of class I molecules. The non-tumorigenic cell line and the cancer cell line are cultured under conditions which allow for expression of the individual soluble class I molecules from the construct, and also allow for endogenous loading of a peptide ligand in the antigen binding groove of each individual soluble class I molecule prior to secretion of the individual soluble class I molecules from the cell. The secreted individual soluble class I molecules having the endogenously loaded peptide ligands bound thereto are isolated from both the non-tumorigenic cell line and the cancer cell line; then, the endogenously loaded peptide ligands are separated from the individual soluble class I molecules from the non-tumorigenic cell, and the endogenously loaded peptide ligands are separated from the individual soluble class I molecules from the cancer cell. The endogenously loaded peptide ligands from the non-tumorigenic cell line and the endogenously loaded peptide ligands from the cancer cell line are then isolated and compared. Finally, at least one endogenously loaded peptide ligand presented by the individual soluble class I molecule in a substantially greater amount on the cancer cell line when compared to the non-tumorigenic cell line is identified.

### Production of Individual, Soluble MHC Molecules

The methods of the presently disclosed and claimed inventive concept(s) may, in one embodiment, utilize a method of producing MHC molecules (from genomic DNA or cDNA) that are secreted from mammalian cells in a bioreactor unit. Substantial quantities of individual MHC molecules are obtained by modifying class I or class II MHC molecules so that they are capable of being secreted, isolated, and purified. Secretion of soluble MHC molecules overcomes the disadvantages and defects of the prior art in relation to the quantity and purity of MHC molecules produced. Problems of quantity are overcome because the cells producing the MHC do not need to be detergent lysed or killed in order to obtain the MHC molecule. In this way the cells producing secreted MHC remain alive and therefore continue to produce MHC. Problems of purity are overcome because the only MHC molecule secreted from the cell is the one that has specifically been constructed to be secreted. Thus, transfection of vectors encoding such secreted MHC molecules into cells which may express endogenous, surface bound MHC provides a method of obtaining a highly concentrated form of the transfected MHC molecule as it is secreted from the cells. Greater purity is assured by transfecting the secreted MHC molecule into MHC deficient cell lines.

Production of the MHC molecules in a hollow fiber bioreactor unit allows cells to be cultured at a density substantially greater than conventional liquid phase tissue culture permits. Dense culturing of cells secreting MHC molecules further amplifies the ability to continuously harvest the transfected MHC molecules. Dense bioreactor cultures of MHC secreting cell lines allow for high concentrations of individual MHC proteins to be obtained. Highly concentrated individual MHC proteins provide an advantage in that most downstream protein purification strategies perform better as the concentration of the protein to be purified increases. Thus, the culturing of MHC secreting cells in bioreactors allows for a continuous production of individual MHC proteins in a concentrated form.

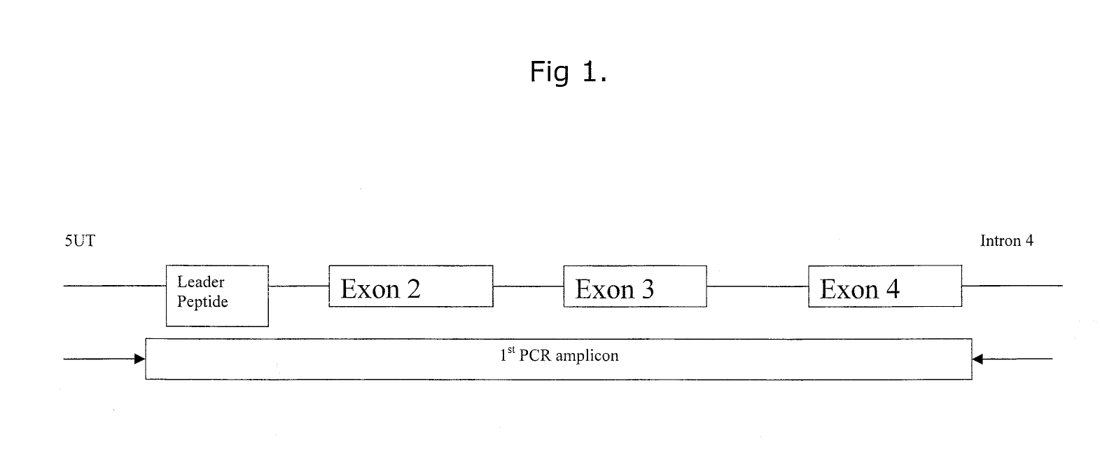

The method of producing MHC molecules utilized in the presently disclosed and claimed inventive concept(s) and described in detail in U.S. Ser. No. 10/022,066 begins by obtaining genomic or complementary DNA which encodes the desired MHC class I or class II molecule. Alleles at the locus which encode the desired MHC molecule are PCR amplified in a locus specific manner. These locus specific PCR products may include the entire coding region of the MHC molecule or a portion thereof. In one embodiment a nested or hemi-nested PCR is applied to produce a truncated form of the class I or class II gene so that it will be secreted rather than anchored to the cell surface. FIG. 1 illustrates the PCR products resulting from such nested PCR reactions. In another embodiment the PCR will directly truncate the MHC molecule.

Locus specific PCR products are cloned into a mammalian expression vector and screened with a variety of methods to identify a clone encoding the desired MHC molecule. The cloned MHC molecules are DNA sequenced to ensure fidelity of the PCR. Faithful truncated clones of the desired MHC molecule are then transfected into a mammalian cell line. When such cell line is transfected with a vector encoding a recombinant class I molecule, such cell line may either lack endogenous class I MHC molecule expression or express endogenous class I MHC molecules. One of ordinary skill of the art would note the importance, given the presently disclosed and claimed inventive concept(s), that cells expressing endogenous class I MHC molecules may spontaneously release MHC into solution upon natural cell death, infection, transformation, etc. In cases where this small amount of spontaneously released MHC is a concern, the transfected class I MHC molecule can be “tagged” such that it can be specifically purified away from spontaneously released endogenous class I molecules in cells that express class I molecules. For example, a DNA fragment encoding a HIS tail may be attached to the protein by the PCR reaction or may be encoded by the vector into which the PCR fragment is cloned, and such HIS tail, therefore, further aids in the purification of the class I MHC molecules away from endogenous class I molecules. Tags beside a histidine tail have also been demonstrated to work, and one of ordinary skill in the art of tagging proteins for downstream purification would appreciate and know how to tag a MHC molecule in such a manner so as to increase the ease by which the MHC molecule may be purified.

Cloned genomic DNA fragments contain both exons and introns as well as other non-translated regions at the 5′ and 3′ termini of the gene. Following transfection into a cell line which transcribes the genomic DNA (gDNA) into RNA, cloned genomic DNA results in a protein product thereby removing introns and splicing the RNA to form messenger RNA (mRNA), which is then translated into an MHC protein. Transfection of MHC molecules encoded by gDNA therefore facilitates reisolation of the gDNA, mRNA/cDNA, and protein. Production of MHC molecules in non-mammalian cell lines such as insect and bacterial cells requires cDNA clones, as these lower cell types do not have the ability to splice introns out of RNA transcribed from a gDNA clone. In these instances the mammalian gDNA transfectants of the presently disclosed and claimed inventive concept(s) provide a valuable source of RNA which can be reverse transcribed to form MHC cDNA. The cDNA can then be cloned, transferred into cells, and then translated into protein. In addition to producing secreted MHC, such gDNA transfectants therefore provide a ready source of mRNA, and therefore cDNA clones, which can then be transfected into non-mammalian cells for production of MHC. Thus, the presently disclosed and claimed inventive concept(s) which starts with MHC genomic DNA clones allows for the production of MHC in cells from various species.

A key advantage of starting from gDNA is that viable cells containing the MHC molecule of interest are not needed. Since all individuals in the population have a different MHC repertoire, one would need to search more than 500,000 individuals to find someone with the same MHC complement as a desired individual—such a practical example of this principle is observed when trying to find a donor to match a recipient for bone marrow transplantation. Thus, if it is desired to produce a particular MHC molecule for use in an experiment or diagnostic, a person or cell expressing the MHC allele of interest would first need to be identified. Alternatively, in the method of the presently disclosed and claimed inventive concept(s), only a saliva sample, a hair root, an old freezer sample, or less than a milliliter (0.2 ml) of blood would be required to isolate the gDNA. Then, starting from gDNA, the MHC molecule of interest could be obtained via a gDNA clone as described herein, and following transfection of such clone into mammalian cells, the desired protein could be produced directly in mammalian cells or from cDNA in several species of cells using the methods of the presently disclosed and claimed inventive concept(s) described herein.

Current methodologies used by others to obtain an MHC allele for protein expression typically start from mRNA, which requires a fresh sample of mammalian cells that express the MHC molecule of interest. Working from gDNA does not require gene expression or a fresh biological sample. It is also important to note that RNA is inherently unstable and is not as easily obtained as is gDNA. Therefore, if production of a particular MHC molecule starting from a cDNA clone is desired, a person or cell line that is expressing the allele of interest must traditionally first be identified in order to obtain RNA. Then a fresh sample of blood or cells must be obtained; experiments using the methodology of the presently disclosed and claimed inventive concept(s) show that >5 milliliters of blood that is less than 3 days old is required to obtain sufficient RNA for MHC cDNA synthesis. Thus, by starting with gDNA, the breadth of MHC molecules that can be readily produced is expanded. This is a key factor in a system as polymorphic as the MHC system; hundreds of MHC molecules exist, and not all MHC molecules are readily available. This is especially true of MHC molecules unique to isolated populations or of MHC molecules unique to ethnic minorities. Starting class I or class II MHC molecule expression from the point of genomic DNA simplifies the isolation of the gene of interest and insures a more equitable means of producing MHC molecules for study; otherwise, one would be left to determine whose MHC molecules are chosen and not chosen for study, as well as to determine which ethnic population from which fresh samples cannot be obtained and therefore should not have their MHC molecules included in a diagnostic assay.

While cDNA may be substituted for genomic DNA as the starting material, production of cDNA for each of the desired HLA class I types will require hundreds of different, HLA typed, viable cell lines, each expressing a different HLA class I type. Alternatively, fresh samples are required from individuals with the various desired MHC types. The use of genomic DNA as the starting material allows for the production of clones for many HLA molecules from a single genomic DNA sequence, as the amplification process can be manipulated to mimic recombinatorial and gene conversion events. Several mutagenesis strategies exist whereby a given class I gDNA clone could be modified at either the level of gDNA or at the cDNA resulting from this gDNA clone. The process of producing MHC molecules utilized in the presently disclosed and claimed inventive concept(s) does not require viable cells, and therefore the degradation which plagues RNA is not a problem.

### Methods of Epitope Discovery and Comparative Ligand Mapping

Peptide epitopes unique to infected and cancerous cells can be directly identified by the methods of the presently disclosed and claimed inventive concept(s), which include producing sHLA molecules in cancerous and infected cells and then sequencing the epitopes unique to the cancerous or infected cells. Such epitopes can then be tested for their binding to various HLA molecules to see how many HLA molecules these epitopes might bind. This direct method of epitope discovery is described in detail in U.S. Ser. No. 09/974,366 and is briefly described herein below.

The method of epitope discovery included in the presently disclosed and claimed inventive concept(s) (and described in detail in U.S. Ser. No. 09/974,366) includes the following steps: (1) providing a cell line containing a construct that encodes an individual soluble class I or class II MHC molecule (wherein the cell line is capable of naturally processing self or nonself proteins into peptide ligands capable of being loaded into the antigen binding grooves of the class I or class II MHC molecules); (2) culturing the cell line under conditions which allow for expression of the individual soluble class I or class II MHC molecule from the construct, with such conditions also allowing for the endogenous loading of a peptide ligand (from the self or non-self processed protein) into the antigen binding groove of each individual soluble class I or class II MHC molecule prior to secretion of the soluble class I or class II MHC molecules having the peptide ligands bound thereto; and (3) separating the peptide ligands from the individual soluble class I or class II MHC molecules.

Class I and class II MHC molecules are really a trimolecular complex consisting of an alpha chain, a beta chain, and the alpha/beta chain's peptide cargo (i.e., the peptide ligand) which is presented on the cell surface to immune effector cells. Since it is the peptide cargo, and not the MHC alpha and beta chains, which marks a cell as infected, tumorigenic, or diseased, there is a great need to identify and characterize the peptide ligands bound by particular MHC molecules. For example, characterization of such peptide ligands greatly aids in determining how the peptides presented by a person with MHC-associated diabetes differ from the peptides presented by the MHC molecules associated with resistance to diabetes. As stated above, having a sufficient supply of an individual MHC molecule, and therefore that MHC molecule's bound peptides, provides a means for studying such diseases. Because the method of the presently disclosed and claimed inventive concept(s) provides quantities of MHC protein previously unobtainable, unparalleled studies of MHC molecules and their important peptide cargo can now be facilitated and utilized to distinguish infected/tumor cells from uninfected/non-tumor cells by unique epitopes presented by MHC molecules in the disease or non-disease state.

The method of the presently disclosed and claimed inventive concept(s) includes the direct comparative analysis of peptide ligands eluted from class I HLA molecules (as described previously in U.S. Publication No. 2002/097672). The teachings of U.S. Publication No. 2002/097672 demonstrates that the addition of a C-terminal epitope tag (such as a 6-HIS or FLAG tail) to transfected class I molecules has no effects on peptide binding specificity of the class I molecule and consequently has no deleterious effects on direct peptide ligand mapping and sequencing, and also does not disrupt endogenous peptide loading.

The method described in parent application U.S. Publication No. 2002/097672 further relates to a novel method for detecting those peptide epitopes which distinguish the infected/tumor cell from the uninfected/non-tumor cell. The results obtained from the present inventive methodology cannot be predicted or ascertained indirectly; only with a direct epitope discovery method can the unique epitopes described therein be identified. Furthermore, only with this direct approach can it be ascertained that the source protein is degraded into potentially immunogenic peptide epitopes. Finally, this unique approach provides a glimpse of which proteins are uniquely up and down regulated in infected/tumor cells.

The utility of such HLA-presented peptide epitopes which mark the infected/tumor cell are three-fold. First, diagnostics designed to detect a disease state (i.e., infection or cancer) can use epitopes unique to infected/tumor cells to ascertain the presence/absence of a tumor/virus. Second, epitopes unique to infected/tumor cells represent vaccine candidates. For example, the presently disclosed and claimed inventive concept(s) describes and claims epitopes which arise on the surface of cells infected with HIV. Such epitopes could not be predicted without natural virus infection and direct epitope discovery. The epitopes detected are derived from proteins unique to virus infected and tumor cells. These epitopes can be used for virus/tumor vaccine development and virus/tumor diagnostics. Third, the process indicates that particular proteins unique to virus infected cells are found in compartments of the host cell they would otherwise not be found in. Thus, uniquely upregulated or trafficked host proteins are identified for drug targeting to kill infected cells. Therefore, the conserved and unique infection/cancer epitopes identified by the methods described herein are useful in the development of antibody and T cell based immunotherapeutics.

While the epitopes detected as unique to infected/tumor cells may serve as direct targets (i.e., through diagnostic, vaccine or therapeutic means), such epitopes may also be utilized to influence the environment around a diseased cell so that these treatments and therapies are effective, and thus allowing the immune responses to see the diseased cell.

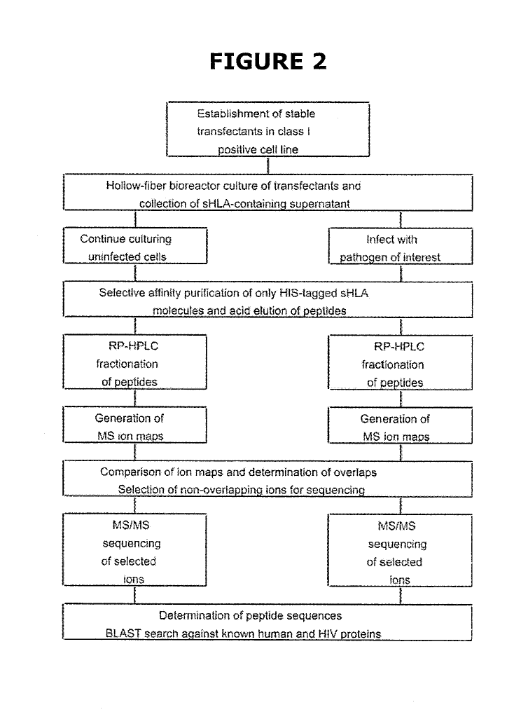

The presently disclosed and claimed inventive concept(s), as well as the parent application U.S. Publication No. 2002/097672, describe, in particular, peptide epitopes unique to HIV infected cells. Peptide epitopes unique to the HLA molecules of HIV infected cells were identified by direct comparison to HLA peptide epitopes from uninfected cells by the method illustrated in the flow chart of FIG. 2. Such method has been shown to be capable of identifying: (1) HLA presented peptide epitopes, derived from intracellular host proteins, that are unique to infected cells but not found on uninfected cells, and (2) that the intracellular source-proteins of the peptides are uniquely expressed/processed in HIV infected cells such that peptide fragments of the proteins can be presented by HLA on infected cells but not on uninfected cells.

The method of epitope discovery and comparative ligand mapping also, therefore, describes the unique expression of proteins in infected cells or, alternatively, the unique trafficking and processing of normally expressed host proteins such that peptide fragments thereof are presented by HLA molecules on infected cells. These HLA presented peptide fragments of intracellular proteins represent powerful alternatives for diagnosing virus infected cells and for targeting infected cells for destruction (i.e., vaccine development).

A group of the host source-proteins for HLA presented peptide epitopes unique to HIV infected cells represent source-proteins that are uniquely expressed in cancerous cells. For example, through using the methodology of the presently disclosed and claimed inventive concept(s) a peptide fragment (SEQ ID NO:12) of reticulocalbin is uniquely found on HIV infected cells. A literature search indicates that the reticulocalbin gene is uniquely upregulated in cancer cells (breast cancer, liver cancer, colorectal cancer). Thus, the HLA presented peptide fragment of reticulocalbin which distinguishes HIV infected cells from uninfected cells can be inferred to also differentiate tumor cells from healthy non-tumor cells. Thus, HLA presented peptide fragments of host genes and gene products that distinguish the tumor cell and virus infected cell from healthy cells have been directly identified. The epitope discovery method is also capable of identifying host proteins that are uniquely expressed or uniquely processed on virus infected or tumor cells. HLA presented peptide fragments of such uniquely expressed or uniquely processed proteins can be used as vaccine epitopes and as diagnostic tools.

The methodology of targeting and detecting virus infected cells is not meant to target the virus-derived peptides. Rather, the methodology of the presently disclosed and claimed inventive concept(s) indicates that the way to distinguish infected cells from healthy cells is through alterations in host encoded protein expression and processing. This is true for cancer as well as for virus infected cells. The methodology according to the presently disclosed and claimed inventive concept(s) results in data which indicates, without reservation, that proteins/peptides distinguish virus/tumor cells from healthy cells.

In a brief example of the methodology of comparative ligand mapping utilized in the methods of the presently disclosed and claimed inventive concept(s), a cell line producing individual, soluble MHC molecules is constructed as described herein before and in US Publication No. 2003/0166057. A portion of the transfected cell line is co-cultured with a virus of interest, resulting in high-titre virus and providing infected cells. In the case of influenza virus, the infection is not productive in the bioreactor and does not result in the production of high titer virus. Because of this, fresh influenza virus was added to the co-culture. In the example provided herein and in detail in US Publication No. 2003/0166057, the viruses of interest are HIV, influenza and WNV. Alternatively, a portion of the cell line producing individual, soluble MHC molecules may be transformed to produce a tumor cell line.

The non-infected cell line and the cell line infected with HIV are both cultured in hollow-fiber bioreactors as described herein above and in detail in US Publication No. 2003/0166057, and the soluble HLA-containing supernatant is then removed from the hollow-fiber bioreactors. The uninfected and infected harvested supernatants were then treated in an identical manner post-removal from the CELL-PHARM®.

MHC class I-peptide complexes were affinity purified from the infected and uninfected supernatants using W6/32 antibody. Following elution, peptides were isolated from the class I molecules and separated by reverse phase HPLC fractionation. Separate but identical (down to the same buffer preparations) peptide purifications were done for each peptide-batch from uninfected and infected cells.

Fractionated peptides were then mapped by mass spectrometry to generate fraction-based ion maps. Spectra from the same fraction in uninfected/infected cells were manually aligned and visually assessed for the presence of differences in the ions represented by the spectra. Ions corresponding to the following categories were selected for MS/MS sequencing: (1) upregulation in infected cells (at least 1.5 fold over the same ion in uninfected cells), (2) downregulation in infected cells (at least 1.5 fold over the same ion in the uninfected cells), (3) presence of the ion only in infected cells, or (4) absence of ion in infected cells that is present in uninfected cells. In addition, multiple parameters were established before peptides were assigned to one of the above categories, including checking the peptide fractions preceding and following the peptide fraction by MS/MS to ensure that the peptide of interest was not present in an earlier or later fraction as well as generation of synthetic peptides and subjection to MS/MS to check for an exact match. In addition, one early quality control step involves examining the peptide's sequence to see if it fits the “predicted motif” defined by sequences that were previously shown to be presented by the MHC molecule utilized.

After identification of the epitopes, literature searches were performed on source proteins to determine their function within the infected cell, and the source proteins were classified into groups according to functions inside the cell. Secondly, source proteins were scanned for other possible epitopes which may be bound by other MHC class I alleles. Peptide binding predictions were employed to determine if other peptides presented from the source proteins were predicted to bind, and proteasomal prediction algorithms were likewise employed to determine the likelihood of a peptide being created by the proteasome.

In accordance with the presently disclosed and claimed inventive concept(s), Table I lists peptide ligands that have been identified as being presented by the B*0702 and A*0201 or B*1801 class I MHC molecule in cells infected with the HIV MN-1 virus but not in uninfected cells, and also lists one peptide ligand that has been identified as not being presented by the B*0702 class I MHC molecule in cells infected with the HIV MN-1 virus that is presented in uninfected cells. One of ordinary skill in the art can appreciate the novelty and usefulness of the present methodology in directly identifying such peptide ligands and the importance such identification has for numerous therapeutic (vaccine development, drug targeting) and diagnostic tools.

As stated above, Table I identifies the sequences of peptide ligands identified to date as being unique to HIV infected cells. Class I sHLA B*0702, A*0201 or B*1801 was harvested from T cells infected and not infected with HIV. Peptide ligands were eluted from B*0702, A*0201 or B*1801 and comparatively mapped on a mass spectrometer so that ions unique to infected cells were apparent. Ions unique to infected cells (and one ligand unique to uninfected cells) were subjected to mass spectrometric fragmentation for peptide sequencing.

TABLE I
Peptides Identified on Infected Cells That Are Not Present on Uninfected Cells
Restricting allele for Sequences marked with a () is HLA-B*0702.
Restricting allele for Sequences marked with a (▪) is HLA-A*0201 or HLA-B*1801.

| | | | | SEQ |
|---|---|---|---|---|
| | | Source | | ID |
| | Sequence | Protein | Category | NO: |
| | EQMFEDIISL | HIV MN-1, ENV | HIV-DERIVED | 1 |
| | IPCLLISFL | Cholinergic Receptor, alpha-3 polypeptide | Signal transduction; ion channel | 2 |
| | STTAICATGL | Ubiquitin-specific protease 3 | Ubiquitin-protease activity; hydrolase | 3 |
| | | | activity | |
| | APAQNPEL | HLA-B associated transcript 3 (BAT3) | MHC gene product | 4 |
| | LVMAPRTVL | HLA-B heavy chain leader sequence | MHC gene product | 5 |
| | APFI[NS]PADX | Unknown, close to several cDNA's | UNKNOWN | 6 |
| | TPQSNRPVm | RNA polymerase II, polypeptide A | DNA binding; protein binding; | 7 |
| | | | transcription | |
| | AARPATSTL | Eukaryotic translation initiation factor 4GI | RNA binding; translation initiation | 8 |
| | | | factor | |
| | MAMMAALMA | Sparc-likek protein 1 | calcium ion binding; extracellular | 9 |
| | | | space | |
| | IATVDSYVI | Tenascin | protein binding; extracellular space | 10 |
| | SPNQARAQAAL | Polypyrimidine tract binding protein 1 | RNA binding | 11 |
| | GPRTAALGLL | Reticulocalbin 2 | calcium ion binding; protein binding | 12 |
| | NPNQNKNVAL | ELAV (HuR) | RNA binding; RNA catabolism | 13 |
| | RPYSNVSNL | Set-binding factor 1 | protein phosphatase activity | 14 |
| | LPQANRDTL | Rac GTPase activating protein 1 | electron transporter; iron binding; | 15 |
| | | | intracellular signalling | |
| | QPRYPVNSV | TCP-1 alpha | ATP binding; chaperone activity | 16 |
| | APAYSRAL | Heat shock protein 27 | protein binding; chaperone | 17 |
| | APKRPPSAF | High mobility group protein 1 or 2 | DNA binding; DNA unwinding | 18 |
| | AASKERSGVSL | Histone H1 family member | DNA binding | 19 |
| ▪ | FIISRTQAL | karyopherin beta 2; importin beta 2; | intracellular protein transport; | 20 |
| | | transportin | nuclear import | |
| ▪ | SLAGSLRSV | FLI00164 protein | no description | 21 |
| ▪ | YGMPRQIL | similar to Homo sapiens mRNA for KIAA0120 | muscle development | 22 |
| | | gene with GenBank Accession Number | | |
| | | D21261.1 | | |
| ▪ | MIIINKFV | hypothetical protein XP_103946 | no description | 23 |
| ▪ | ALWDIETGQQTV | G protein beta subunit | GTPase activity; signal transducer | 24 |
| ▪ | VLMTEDIKL | eukaryotic translation initiation factor 4 | calcium ion binding; extracellular | 25 |
| | | gamma, 1 | space | |
| ▪ | YIYDKDMEII | usp22 | Ubiquitin-protease activity; hydrolase | 26 |
| | | | activity | |
| ▪ | ALMPVLNQV | homolog of yeast mRNA transport regulator | exosome constituent | 27 |
| | | 3 | | |
| ▪ | DLIIKGISV | TAR DNA binding protein | RNA binding; transcription factor | 28 |
| | | | activity | |
| ▪ | QLVDIIEKV | proteasome activator 28-gamma; 11S | proteasome activator activity | 29 |
| | | regulator complex gamma subunit; | | |
| | | proteasome activator subunit 3 isoform 2; Ki | | |
| | | nuclear autoantigen | | |
| ▪ | IMLEALERV | snRNP polypeptide G | RNA binding; RNA splicing; | 30 |
| | | | spliceosome assembly | |
| ▪ | DAYIRIVL | engulfment and cell motility 1 isoform 1; | signal transduction; cell motility | 31 |
| | | ced-12 homolog 1 | | |
| ▪ | ILDPHVVLL | nucleoporin 88kDa | transporter activity; nuclear pore | 32 |
| | | | transport | |
| ▪ | DAKIRIFDL | laminin receptor homolog or ribosomal | ribosome constituent | 33 |
| | | protein L10 | | |
| ▪ | ALLDKLYAL | brms2 or mitochondrial ribosomal protein S4 | RNA binding; ribosome constituent | 34 |
| | | or | | |
| ▪ | FMFDEKLVTV | serine/threonine protein phosphatase | hydrolase activity; manganese ion | 35 |
| | | catalytic subunit | binding | |
| ▪ | SLAQYLINV | hnRNP E2 | DNA binding; RNA binding | 36 |
| ▪ | SLLQTLYKV | Similar to RAN GTPase activating protein 1 | GTPase activator activity; signal | 37 |
| | | | transducer | |
| ▪ | YMAELIERL | Geminin | cell cycle; DNA replication inhibitor | 38 |
| ▪ | FLYLIIISY | HIV-1 TAR RNA-binding protein B | no description | 39 |
| ▪ | SLLENLEKI | hnrnpC1/C2 | MHC gene product | 40 |
| ▪ | FLFNKVVNL | yippee protein | no description | 41 |
| ▪ | VLWDRTFSL | STAT-1 | transcription factor activity; signal | 42 |
| | | | transduction | |
| ▪ | SLASVFVRL | Similar to histone deacetylase 4 | no description | 43 |
| ▪ | FLMDFIHQV | Nuclear pore complex protein Nup133 | transporter activity; nuclear pore | 44 |
| | | (Nucleoporin Nup133) | transport | |
| ▪ | FLWDEGFHQL | glucosidase I | carbohydrate metabolism | 45 |
| ▪ | TALPRIFSL | TAP | ABC transporter | 46 |
| ▪ | KLWEMDNMLI | T-cell activation protein | ribosome constituent | 47 |
| ▪ | MVDGTLLLL | HLA-E leader sequence | MHC gene product | 48 |
| ▪ | SLLDEFYKL | membrane component, chromosome 11, | integral to plasma membrane | 49 |
| | | surface marker 1 | | |
| ▪ | YLLPAIVHI | P68 RNA helicase | ATP binding; RNA binding; RNAhelicase | 50 |
| | | | activity | |
| ▪ | SLASLHPSV | PLAG-LIKE 1 or ZAC delta 2 protein or zinc | nucleic acid binding; zinc ion | 51 |
| | | finger protein or lost on transformation LOT1 | binding | |
| ▪ | KLWDIINVNI | steroid-dehydrogenase like | oxidoreductase activity; metabolism | 52 |
| ▪ | KYPENFFLL | protein phosphatase I | protein phosphatase activity | 53 |
| ▪ | YLLIEEDIRDLAA | TdT binding protein | TdT binding | 54 |
| ▪ | DELQQPLEL | signal transducer and activator of | transcription factor activity; signal | 55 |
| | | transcription 2; signal transducer and | transduction | |
| | | activator of transcription 2, 113 kD; | | |
| | | interferon alpha induced transcriptional | | |
| | | activator | | |
| ▪ | DEYEKLQVL | Dynein heavy chain, cytosolic (DYHC) | ATP binding; nucleic acid binding; | 56 |
| | | (Cytoplasmic dynein heavy chain 1) (DHC1) | mitotic spindle assembly | |
| ▪ | EEYQSLIRY | Protein CGI-126 (Protein HSPC155) | ubiquitin-conjugating enzyme activity | 57 |
| ▪ | DDWKVIANY | c-myb protein | DNA binding | 58 |
| ▪ | DELLNKFV | adaptor-related protein complex 2, alpha 1 | protein transporter | 59 |
| | | subunit isoform 1; adaptin, alpha A; | | |
| | | clathrin-associated/assembly/adaptor | | |
| | | protein | | |
| ▪ | DEFKVVVV | COPG protein | vesicle coat complex | 60 |
| ▪ | LEGLTVVY | CGI-120 protein; likely ortholog of mouse | protein transporter activity | 61 |
| | | coatomer protein complex, subunit zeta 1 | | |
| ▪ | VEEILSVAY | RNA helicase II/Gu protein | ATP binding; RNA binding | 62 |
| ▪ | DEDVLRYQF | cyclophilin 60kDa; peptidylprolyl isomerase- | isomerase activity; protein folding | 63 |
| | | like 2 isoform b; cyclophilin-like protein | | |
| | | CyP-60; peptidylprolyl cis-trans isomerase; | | |
| ▪ | DEGTAFLVY | butyrylcholinesterase precursor | enzyme binding; hydrolase activity | 64 |
| ▪ | MEQVIFKY | ARP3 actin-related protein 3 homolog; ARP3 | constituent of cytoskeleton; cell | 65 |
| | | (actin-related protein 3, yeast) homolog | motility | |
| ▪ | NEQAFEEVF | replication protein A1, 70 kDa; replication | DNA binding; DNA recombination | 66 |
| | | protein A1 (70 kD) | | |
| ▪ | VEEYVYEF | heat shock 105 kD; heat shock 105 kD alpha; | ATP binding; chaperone activity | 67 |
| | | heat shock 105 kD beta; heat shock 105 kDa | | |
| | | protein 1 | | |
| ▪ | DEIQVPVL | rab3-GAP regulatory domain | GTPase activator; intracellular | 68 |
| | | | protein transporter | |
| ▪ | DEYQFVERL | mitochondrial ribosomal protein L49; | structural constituent of ribosomes | 69 |
| | | neighbor of FAU; next to FAU [Homo sapiens] | | |
| ▪ | DEYSIFPQTY | ras-related GTP-binding protein | GTP binding; signal tranducer | 70 |
| ▪ | DEYSLVREL | talin | actin binding; cytoskeleton | 71 |
| ▪ | EEVETFAF | HSP 90 | chaperone activity | 72 |
| ▪ | NENDIRVMF | elav-type RNA-binding protein; RNA-binding | RNA binding; RNA processing | 73 |
| | | protein BRUNOL3 | | |
| ▪ | DEYDFYRSF | polymyositis/scleroderma autoantigen 2, | RNA binding; hydrolase activity | 74 |
| | | 100 kDa; autoantigen PM-SCL; | | |
| | | polymyositis/scleroderma autoantigen 2 | | |
| | | (100 kD) | | |
| ▪ | DEFQLLQAQY | AES-1 or AES-2 | transcription factor activity | 75 |
| ▪ | DEFEFLEKA | zinc finger protein 147 (estrogen-responsive | transcription factor activity | 76 |
| | | finger protein) | | |
| ▪ | DEMKVLVL | beta-fodrin | actin binding | 77 |
| ▪ | DERVFVALY | similar to source of immunodominant MHC- | no description | 78 |
| | | associated peptides | | |
| ▪ | IENPFGETF | integral inner nuclear membrane protein | integral to inner nuclear membrane | 79 |
| ▪ | SEFELLRSY | sorting nexin 4 | protein transporter; intracellular | 80 |
| | | | signaling | |
| ▪ | DEGRLVLEF | Acyl-coA/cholesterol acyltransferase | no description | 81 |
| ▪ | DEGWFLIL | RNA helicase family | ATP binding; nucleic acid binding; | 82 |
| | | | hydrolase activity | |
| ▪ | DEISFVNF | structure specific recognition protein 1; | DNA binding; transcription regulator | 83 |
| | | recombination signal sequence recognition | activity | |
| | | protein; chromatin-specific transcription | | |
| | | elongation factor 80 kDa subunit | | |
| ▪ | SEVLSWQF | signal transducer and activator of | transcription factor activity; signal | 84 |
| | | transcription-1; | transduction | |
| ▪ | YEILLGKATLY | T cell receptor beta-chain | MHC binding; receptor activity | 85 |
| ▪ | YENLLAVAF | unnamed protein product | protein modification | 86 |
| ▪ | DETQIFSYF | nucleolar phosphoprotein Nopp34 | RNA binding; protein binding | 87 |
| ▪ | MEPLRVLEL | DNA methyltransferase 2 isoform d; DNA | DNA binding; DNA methylation | 88 |
| | | methyltransferase-2; DNA methyltransferase | | |
| | | homolog HsaIIP; DNA MTase homolog HsallP | | |
| ▪ | MPLGKTLPC | laminin | protein binding; structural molecule | 89 |
| | | | activity | |
| ▪ | VYMDWYEKF | U5 snrnp 200 kDa helicase | ATP binding; nucleic acid binding; | 90 |
| | | | RNA splicing | |
| ▪ | SELLIHVF | protein kinase c-iota | ATP binding; protein binding | 91 |
| ▪ | DEHLITFF | U5 snrnp 200 kDa helicase | ATP binding; nucleic acid binding; | 92 |
| | | | RNA splicing | |
| ▪ | DEFKIGELF | DNA-PKcs | DNA binding; transferase activity | 93 |
| ▪ | DELEIIEGMKF | (Heat shock protein 60) (HSP-60) | ATP binding; chaperone activity | 94 |
| ▪ | KYLLSATKLR | melanoma-derived leucine zipper, extra- | no description | 95 |
| | | nuclear factor | | |
| ▪ | SEIELFRVF | U5 small nuclear ribonucleoprotein 200 kDa | ATP binding; nucleic acid binding; | 96 |
| | | helicase | sRNA plicing | |
| ▪ | LEDVLPLAF | HP1-BP74 | DNA binding; nucleosome assembly | 97 |

In order to provide an analysis of peptides after HIV-infection under as-close-as possible conditions as those that would occur inside an infected person, a human T cell line was utilized for infection with HIV. This cell line, Sup-T1, possesses its own class I; HLA-A and -B types are A*2402, A*6801, B*0801, and B*1801. Although only the soluble class I specifically introduced into the cell should be secreted, under some conditions shedding of full-length class I molecules has been observed. It is believed that HLA-B*1801 is shed after HIV infection.

Analysis of soluble A*0201 produced a number of ligands that did not appear to fit the A*0201 peptide motif (an indication of which amino acids are preferred at particular positions of the peptide). For instance, A*0201 prefers peptides with an L at position 2 (P2) and an L or V at P9. Most of the peptides that did not match the A*0201 motif had an E at P2 and a Y or F at P9.

Upon inspection, these peptides were most likely derived from B*1801. To confirm, several peptides from B*1801 molecules in a class I negative cell line were sequenced, and several overlapping peptides were identified. Therefore, at this point, the peptides are labeled as either A*0201 or B*1801 restricted. Tests are currently being performed to delineate which of the two molecules binds each peptide. However, simple analysis of the peptide sequence (P2 and P9 amino acids) should be sufficient to determine the restricting molecule, and such simple analysis is within the ability of a person having ordinary skill in the art.

The methodology used herein is to use sHLA to determine what is unique to unhealthy cells as compared to healthy cells. Using sHLA to survey the contents of a cell provides a look at what is unique to unhealthy cells in terms of proteins that are processed into peptides. The data summarized in TABLE I shows that the epitope discovery technique described herein is capable of identifying sHLA bound epitopes and their corresponding source proteins which are unique to infected/unhealthy cells.

Likewise, peptide ligands presented in individual class I MHC molecules in an uninfected cell that are not presented by individual class I MHC molecules in an uninfected cell can also be identified. The peptide “GSHSMRY” (SEQ ID NO:98), for example, was identified by the method of the presently disclosed and claimed inventive concept(s) as being an individual class I MHC molecule which is presented in an uninfected cell but not in an infected cell. The source protein for this peptide is MHC Class I Heavy Chain, which could be derived from multiple alleles, i.e., HLA-B*0702 or HLA-G, etc.

The utility of this data is at least threefold. First, the data indicates what comes out of the cell with HLA. Such data can be used to target CTL to unhealthy cells. Second, antibodies can be targeted to specifically recognize HLA molecules carrying the ligand described. Third, realization of the source protein can lead to therapies and diagnostics which target the source protein. Thus, an epitope unique to unhealthy cells also indicates that the source protein is unique in the unhealthy cell.

The methods of epitope discovery and comparative ligand mapping described herein are not limited to cells infected by a microorganism such as HIV. Unhealthy cells analyzed by the epitope discovery process described herein can arise from virus infection or also from cancerous transformation. Unhealthy cells may also be produced following treatment of healthy cells with a cancer causing agent, such as but not limited to, nicotine, or by a disease state cytokine such as IL-4. In addition, the status of an unhealthy cell can also be mimicked by transfecting a particular gene known to be expressed during viral infection or tumor formation. For example, particular genes of HIV can be expressed in a cell line as described (Achour, A., et al., AIDS Res Hum Retroviruses, 1994. 10(1): p. 19-25; and Chiba, M., et al., CTL. Arch Virol, 1999. 144(8): p. 1469-85, all of which are expressly incorporated herein by reference) and then the epitope discovery process performed to identify how the expression of the transferred gene modifies epitope presentation by sHLA. In a similar fashion, genes known to be upregulated during cancer (Smith, E. S., et al., Nat Med, 2001. 7(8): p. 967-72, which is expressly incorporated herein by reference) can be transferred in cells with sHLA and epitope discovery then completed. Thus, epitope discovery with sHLA as described herein can be completed on cells infected with intact pathogens, cancerous cells or cell lines, or cells into which a particular cancer, viral, or bacterial gene has been transferred. In all these instances the sHLA described here will provide a means for detecting what changes in terms of epitope presentation and the source proteins for the epitopes.

The methods of the presently disclosed and claimed inventive concept(s) have also been applied to identifying epitopes unique or upregulated in influenza infected cells as well as West Nile virus infected cells. The methods for obtaining soluble HLA form cells infected with Influenza and West Nile Virus (WNV) are similar to those described hereinabove for HIV infection, except as described herein below. During the course of both the Influenza and WNV infection in the bioreactor, the viral infection was monitored to ensure that the cells secreting the HLA molecules were infected. For Influenza, this was accomplished by measuring intracellular infection using antibody staining combined with flow cytometry. For West Nile virus (WNV), this was accomplished by: (1) measuring viral titer in supernatant using reverse transcriptase real-time PCR; and/or (2) measuring intracellular infection using antibody staining and fluorescence in situ hybridization combined with flow cytometry.

Table II lists unique and upregulated peptide epitopes that have been identified by the A*0201 and B*0702 class I MHC molecules in cells infected with the PR8 strain of influenza A virus.

Table III lists unique peptide epitopes that have been identified by the A*0201 class I MHC molecules in cells infected with the West Nile virus. Both self and viral epitopes have been identified.

TABLE II
Peptides Identified on Influenza - Infected Cells.
PR8 A0201

| | | Fold | | SEQ ID |
|---|---|---|---|---|
| Peptide | Source Protein | Increase | Gene | NO: |
| NDHFVKL | Uracil DNA glycosylase/GAPDH | 7.75 | GAPDH | 99 |
| GLMTTVHAIT | Uracil DNA glycosylase/GAPDH | 2.5 | GAPDH | 100 |
| ALNDHFVKL | Uracil DNA glycosylase/GAPDH | 23.02 | GAPDH | 101 |
| RLTPKLMEV | eIF3-gamma | 2.2 | EIF3S3 | 102 |
| KLEEIIHQI | Hypothetical protein | 2.08 | | 103 |
| KLLEGEESRISL | Vimentin | 2.1 | VIM | 104 |
| ALNEKLVNL | eIF3-epsilon | 1.52 | EIF3S5 | 105 |
| LLDVPTAAV | GILT | 5.18 | IF130 | 106 |
| AVGKVIPEL | Uracil DNA glycosylase/GAPDH | 12.46 | GAPDH | 107 |
| GLMTTVHAITA | Uracil DNA glycosylase/GAPDH | 3.2 | GAPDH | 108 |
| TLAEVERLKGL | U2 snRNP | Unique | SNRPA1 | 109 |
| GLMTTVHAITATQ | Uracil DNA glycosylase/GAPDH | Unique | GAPDH | 110 |
| GVLDNIQAV | Histone | Unique | HIST1H2AE | 111 |
| ALDKATVLL | Programmed cell death 4 isoform 2 | 2.13 | PDCD4 | 112 |
| KVPEWVDTV | Ribosomal protein S19 | 5.94 | RPS19 | 113 |
| KMLEKLPEL | ABCF3 protein | 2.14 | ABCF3 | 114 |
| FLGRINEI | Suppressor of K+ transport defect-3 | 1.99 | CLPB | 115 |
| GLIEKNIEL | DNA methyl transferase | 1.58 | DNMT1 | 116 |
| KVFDPVPVGV | DEAH box polypeptide 9 | 1.74 | DHX9 | 117 |
| GLMTTVHAITAT | Uracil DNA glycosylase/GAPDH | Unique | GAPDH | 118 |
| FAITAIKGV | ribosomal protein S18 | 3.49 | RPS18 | 119 |
| SMTLAIHEI | Sphingolipid delta 4 desaturase protein DES1 | 2.11 | DEGS1 | 120 |
| LLDANLNIKI | KIAA0999 | 2.78 | | 121 |
| TLWDIQKDLK | Lactate dehydrogenase | 1.64 | LDHB | 122 |
| KMYEEFLSKV | c-AMP dependent protein kinase type 1-beta | 1.8 | PRKAR1B | 123 |
| | regulatory subunit | | | |
| FLASESLIKQIPR | Ribosomal Protein L10a | Unique | RPL10A | 124 |
| KLFDDDETGKISF | Caltractin | Unique | CETN2 | 125 |
| SLDQPTQTV | eIF3 subunit 8 | 9.84 | ElF3S8 | 126 |
| GIDSSSPEV | poly(rc) binding protein | Unique | PCBP1 | 127 |
| KAPPAPLAA | Inner nuclear membrane protein | Unique | MAN1 | 128 |
| ILDKKVEKV | HSP90 | Unique | HSP90AB1 | 129 |
| KLDEGNSL | DNA topisonnerase II | 4.32 | TOP2A | 130 |
| VVQDGIVKA | Peroxiredoxin 5 | Unique | PRDX5 | 131 |
| ALGNVRTV | Unknown protein | | | 132 |
| YLEAGGTKV | Homolog of yeast mRNA Transport Regulator | | | 133 |
| ALSDGVHKI | Fas apoptotic inhibitory molecule | 1.88 | FAIM | 134 |
| GLAEDSPKM | Chromosome 17 open reading frame 27 | 2 | c17orf27 | 135 |
| EAAHVAEQL | MHC A2 antigen | | | 136 |
| AQAPDLQRV | Noll | | NOL1 | 137 |
| GVYGDVHRV | Rod 1 regulator of differentiation | 2.9 | ROD1 | 138 |
| YLTHDSPSV | sNRPC | | snRPC | 139 |
| RLDDVSNDV | Heat repeat containing 2 | 2.55 | HEATR2 | 140 |
| KLMELHGEGSS | Ribosomal protein S3A | Unique | | 141 |
| KMWDPHNDPNA | U1 small ribonucleoprotein 70 kDa | Unique | SNRP70 | 142 |
| ALSDGVHKI | Fas apoptotic inhibitory molecule | 2.36 | FAIM | 143 |
| KLDPTKTTL | n-Myc downstream regulated gene 1 | 2.93 | DRG1 | 144 |
| RVPPPPPIA | hnRPC | 6.54 | HNRPC | 145 |
| FIQTQQLHAA | Pyruvate kinase | Unique | PKM2 | 146 |
| SLTGHISTV | Pleiotropic Regulator 1 | 3.12 | PLRG1 | 147 |
| KIAPNTPQL | Pm5 protein | 2.63 | PM5 | 148 |
| NLDPAVHEV | ATP(GTP) binding protein | | XAB1 | 149 |
| NMVAKVDEV | Ribosomal protein Ll0a | | | 150 |
| YLEDSGHTL | Peroxiredoxin 4 | | PRDX4 | 151 |
| TLDEYTTRV | Nuclear respiratory factor 1 | 3.74 | NRF1 | 152 |
| TLYEHNNEL | AAAS | | AAAS | 153 |
| GLATDVQTV | Proteasome subunit HsC 10-II | 3.5 | PSMB3 | 154 |
| QLLGSAHEV | Non-erythroid alpha-spectrin | 4.98 | SPTAN1 | 155 |
| GLDKQIQEL | ATP dependent 26s proteasome regulatory subunit | 4.09 | PSMC3 | 156 |
| YAYDGKDYIA | MHC-B antigen | 1.6 | | 157 |
| AVSDGVIKV | Cofilin 1 | 8.98 | CFL1 | 158 |
| VLEDPVHAV | Hypothetical protein | 3.91 | | 159 |
| VMDSKIVQV | Karyopherin alpha 1 | 22.84 | KPNA5 | 160 |
| ILGYTEHQV | GAPDH | 23.91 | GAPDH | 161 |
| SMMDVDHQI | Chaperonin containing TCP-1 subunit 5 | 3.58 | CCT5 | 162 |
| YAYDGKDYI | MHC-B antigen | Unique | | 163 |
| LMTTVHAITAT | GAPDH | Unique | GAPDH | 164 |
| AIVDKVPSV | Coatomer protein complex subunit gamma 1 | 1.88 | COPG | 165 |
| SLAKIYTEA | H1 histone family member X | 5.38 | H1FX | 166 |
| SMLEDVQRA | RNA binding motif protein 28 | 2.4 | RBM28 | 167 |
| VLLSDSNLHDA | Cytokine induced apoptosis inhibitor 1 | 10.95 | CIAPIN1 | 168 |
| YLDKVRALE | Keratin | Unique | KRT1 | 169 |
| LLDVVHPA | TCP-1 | 33.09 | CCT7 | 170 |
| LLDVVHPAA | TCP-1 | 3.43 | CCT7 | 171 |
| ALASHLIEA | EH domain containing 2 | 1.67 | EHD2 | 172 |
| ALMDEVVKA | Phosphoglycerate kinase | 2.59 | PGK1 | 173 |
| ILSGVVTKM | Ribosomal protein Sll | 1.74 | RPS11 | 174 |
| ILMEHIHKL | Ribosomal protein L19 | 5.46 | RPL19 | 175 |
| YMEEIYHRI | Farnesyl-diphosphate farnesyltransferase | 3.98 | FDFT1 | 176 |
| FLLEKGYEV | GDP-mannose-4,6-dehydratase | 1.81 | GMDS | 177 |
| TLLEDGTFKV | NmrA-like family domain | 1.67 | NMRAL1 | 178 |
| GLGPTFKL | BBS1 protein | unique | BBS1 | 179 |
| GLIDGRLTI | SPCS2 protein | 1.67 | SPCS2 | 180 |
| ALDEKLLNI | CPSF | 1.61 | CPSF3 | 181 |
| VLMTEDIKL | eIF4G | 1.69 | EIF4G | 182 |
| SLYEMVSRV | SSRP1 | 1.87 | SSRP1 | 183 |
| TLAEIAKVEL | p54nrb | 3.32 | NONO | 184 |
| GLDIDGIYRV | ARHGAP12 protein | 1.95 | ARHGAP12 | 185 |
| LLLDVPTAAVQA | GILT | 6.24 | IF130 | 186 |
| AIIGGTFTV | ERGIC1 | 4.17 | ERGIC1 | 187 |
| GMASVISRL | Tubulin gamma complex associated protein 2 | Unique | TUBGCP2 | 188 |
| TIAQLHAV | Unknown protein | Unique | | 189 |
| RLWPKIQGL | Unknown protein | Unique | | 190 |
| ALQELLSKGL | similar to 40s ribosomal protein s25 | 2.8 | RPS25 | 191 |
| TLWGIQKEL | Lactate dehydrogenase | 3.27 | LDHA | 192 |
| TLWPEVQKL | STATIP1(signal transducer and activator of | 2.97 | STATIP1 | 193 |
| | transcription 3 interacting protein 1) | | | |
| FLFNTENKL | Isopentenyl-diphosphate-delta-isomerase 1 | 1.85 | IDI1 | 194 |
| ALLSAVTRL | Alpha catenin | Unique | CTNNA1 | 195 |
| SLLEKSLGL | eukaryotic translation elongation factor 1 epsilon 1 | 1.64 | EEF1E1 | 196 |
| KIADFGWSV | Aurora kinase C | 2.26 | AURKC | 197 |
| KLQEFLQTL | Unknown protein | 2.3 | | 198 |
| ALWEAKEGGLL | Hypothetical protein | 1.54 | | 199 |
| KLIGDPNLEFV | Ras-related nuclear protein | 2.82 | RAN | 200 |
| GLIENDALL | Unknown protein | 1.71 | | 201 |
| GLAKLIADV | Flap structure-specific endonuclease 1 | 2.91 | FEN1 | 202 |
| TLIGLSIKV | Hypothetical protein | 2.28 | | 203 |
| LLLDVPTAAV | GILT | 1.95 | IF130 | 204 |
| IMLEALERV | SNRPG | 1.64 | SNRPG | 205 |
| TLIDLPGITKV | Dynamin | 6.48 | DNM2 | 206 |
| ALLAGSEYLKL | eIF3 zeta | 1.51 | EIF3S7 | 207 |
| KIIDEDGLLNL | replication factor C Irg subunit | 1.56 | LLDBP | 208 |
| TLQEVFERATF | Nucleolin | Unique | NCL | 209 |
| RLIDLGVGL | Hypothetical protein | 2.03 | | 210 |
| GIVEGLMTTV | Uracil DNA glycosylase | 3.1 | HNG | 211 |
| SMPDFDLHL | AHNAK nucleoprotein isoform 1 | 1.83 | AHNAK | 212 |
| VLFDVTGQVRL | Major vault protein | 2.48 | MVP | 213 |
| FLAEEGFYKF | Integral membrane protein 1 | 2.98 | STT3A | 214 |
| ALVSSLHLL | Coatomer protein complex subunit gamma 1 | 1.51 | IMP3 | 215 |
| ALLDKLYAL | U3 snoRNP protein 3 homolog | 3.1 | | 216 |
| GMYVFLHAV | ORMDL1 protein | 2.73 | ORMLD1 | 217 |
| AMIELVERL | DIPB protein | 1.81 | TRIM44 | 218 |
| VINDVRDIFL | TFIIA | 1.71 | GTF2A1 | 219 |
| FMFDEKLVTV | Protein phosphatase 6 | 1.99 | PPP6C | 220 |
| GVAESIHLWEV | WDR18 | 2.89 | WDR18 | 221 |
| GMYIFLHTV | ORM1-like 3 | 2.32 | ORMDL3 | 222 |
| GLLDPSVFHV | Noc4L protein | 2.17 | NOC4L | 223 |
| GLWDKFSEL | human retinoic acid receptor gamma bound | 2.59 | RARB | 224 |
| KLLDFGSLSNL | 40s ribosomal protein S17 | 3.57 | RPS17 | 225 |
| RLYPWGVVEV | Septin 2 | 2.79 | (SEPT2) | 226 |
| KLFPDTPLAL | ILF3 | Unique | ILF3 | 227 |
| GLQDFDLLRV | Protein kinase C iota | 2.29 | | 228 |
| ILYDIPDIRL | Phenylalanyl-tRNA synthetase alpha chain | 5.99 | FARS1 | 229 |
| LLDVTPLSL | HSP 70 | 9.68 | HSPA2 | 230 |
| TLAKYLMEL | Cyclin B1 | 6.81 | | 231 |
| ALVEIGPRFVL | Brix | 10.83 | BRIX | 232 |
| GIWGFIKGV | Hypothetical protein | 6.1 | | 233 |
| ILCPMIFNL | Unnamed protein product | 2.51 | | 234 |
| FLPSYIIDV | CPSF-1 | 2.57 | CPSF1 | 235 |
| NLAEDIMRL | Vimentin | 2.02 | VIM | 236 |
| YLDIKGLLDV | Skp1 | 2.44 | SKP1A | 237 |
| IIMLEALERV | SNRPG | 13.68 | SNRPG | 238 |
| SIIGRLLEV | Protein phosphatase 1 catalytic subunit alpha 1 | 56.92 | | 239 |
| SLLDIIEKV | Tuberin | 2.56 | TSC2 | 240 |
| KIFEMGPVFTL | Cytochrome C oxidase subunit II | 6.45 | COX2 | 241 |
| GVIAEILRGV | Serine hyroxymethyltransferase | 1.56 | SHMT2 | 242 |
| SLWSIISKV | Transmembrane protein 49EG | 3.06 | TMEM49/ | 243 |
| | | | TDC1 | |
| SLFEGTWYL | 3-hydroxy-3-methylglutaryl CoA synthase | 2.36 | HMGCS1 | 244 |

PR8 B0702

| | | | | |
|---|---|---|---|---|
| RPKANSA | Unknown protein product | 1.8 | | 245 |
| APRPPPKM | Ribosomal protein S26 | 2.9 | | 246 |
| KPQDYKKR | Catenin beta-1 | 2.9 | | 247 |
| RPTGGVGAV | Hydroxymethyl glutanyl CoA synthase | 2.7 | | 248 |
| ARPATSL | eIF4G | 2.2 | | 249 |
| NLGSPRPL | Tripeptidyl peptidase II | 5.6 | | 250 |
| AARPATSTL | eIF4G | 5.1 | | 251 |
| RPGLKNNL | Unknown protein product | 1.5 | | 252 |
| SPGPPTRKL | c14orf12 | 1.9 | | 253 |
| IPSIQSRGL | Influenza A/PR8/34 Hemagglutinin | 1.6 | | 254 |
| LPFDRTTVM | Influenza A/PR8/34 Nucleoprotein | 1.3 | | 255 |
| GPPGTGKTAL | TATA binding protein interacting protein | 1.5 | RPS2 | 256 |
| APRGTGIVSA | RPS2 protein | 2.2 | RPL8 | 257 |
| APAGRKVGL | RPL8 protein | 1.5 | NGRN | 258 |
| APGAPPRTL | Mesenchymal stem cell protein | 1.5 | | 259 |
| APPPPPKAL | MHC HLA B associated transcript 2 | 2.29 | BAG3 | 260 |
| LPSSGRSSL | BAG family molecular chaperone regulator 3 | 2 | FBXL6 | 261 |
| LPKPPGRGV | FBOX protein Fb16 | 1.9 | | 262 |
| NLPLSNLAI | Phosphatidylinositol phospholipase X domain | 4.3 | TYMS | 263 |
| | containing 2 | | | |
| EPRPPHGEL | Thymidylate Synthase | 2.7 | | 264 |
| APNRPPAAL | MHC antigen | 1.5 | HMGB1 | 265 |
| APKRPPSAF | HMG213 | 1.82 | TERF2 | 266 |
| SPPSKPTVL | Telomeric repeat factor 2 | 1.9 | CDKN1C | 267 |
| APRPVAVAV | p57 KIP2 | 1.5 | MCL1 | 268 |
| RPPPIGAEV | MC-1 delta SITM | 2.9 | CPNE3 | 269 |
| RPAGKGSITI | Copine III | 1.8 | GH2 | 270 |
| SPGIPNPGAPL | hGH-V2 human growth factor hormone variant | 1.84 | RUVBL1 | 271 |
| RPQGGQDIL | TATA binding protein interacting protein | 2.24 | ATP5J | 272 |
| PKFEVIEKPQA | ATP synthase H+Transporting mitochondrial FO | 3.6 | | 273 |
| | complex subunit F6 isoform A precursor | | | |
| VFLKPWI | Hypothetical protein | 1.62 | SCD | 274 |
| ITAPPSRVL | SCD Protein | 1.98 | | 275 |
| TPEQIFQN | Hypothetical protein | 1.51 | TGIF2 | 276 |
| LPRGSSPSVL | TGFB-induced factor 2 | 1.57 | | 277 |
| GPREAFRQL | SCAN related protein RAZ | 6.03 | | 278 |
| KPVIKKTL | Hypothetical protein | U | | 279 |
| SPRSGLIRV | glycyl-tRNA synthetase | 1.53 | SMG1 | 280 |
| LLPGENINLL | PI-3 kinases related kinase | 7.13 | | 281 |
| HLNEKRRF | HPV-18 E6 Protein | 2.02 | | 282 |
| TQFVRFDSD | MHC I antigen | 1.64 | DYNC1H1 | 283 |
| RVEPLRNEL | Dynein | 1.95 | | 284 |
| YQFTGIKKY | HCV F-Transactivated Protein 2 | 2.3 | SF3B3 | 285 |
| GPRSSLRVL | Splicing factor 3B subunit 3 | 3.16 | HNRPL | 286 |
| GPYPYTL | Human hnRPL protein | 2.01 | SND1 | 287 |
| SPAKIHVF | 100 kDA coativator | 2.8 | SRP9 | 288 |
| DPMKARVVL | SRP9 protein | 1.87 | | 289 |
| SPQEDKEVI | Novel protein | 4.19 | CLTC | 290 |
| NPASKVIAL | Clathrin heavy chain I | 1.64 | | 291 |
| RPSGKGIVEF | human mRNA gene product | 13.7 | | 292 |
| SPVPSRPL | putative GTP-binding protein Ray-like variant | 2.91 | ACTG1 | 293 |
| APEEHPVLL | Actin-like Protein | 1.92 | | 294 |
| SPKIRRL | Similar to putative membrane bound dipeptidase | 21.63 | PFKM | 295 |
| LVFQPVAEL | Phosphofructokinase | 4.33 | CDADR | 296 |
| GPLDIEWLI | Coxsackie-adenovirus receptor isoform CA R217 | 2.2 | | 297 |
| RIVPRFSEL | Unknown protein product | 1.54 | DDX3X | 298 |
| YPKRPLLGL | DEAD box polypeptide 24 variant | 1.61 | UBE2D3 | 299 |
| YPFKPPKVAF | Ubiquitin conjugating enzyme 1 | 3.27 | RPL12 | 300 |
| APKIGPLGL | 60s Ribosomal protein L12 LIKE protein | 1.54 | | 301 |

TABLE III
Peptides Identified on West Nile Virus Infected Cells.

| | | | | |
|---|---|---|---|---|
| | | | Fold | SEQ ID |
| Species | Sequence | Protein | increase | NO: |

SELF EPIOPES

| | | | | |
|---|---|---|---|---|
| Human | AVLDELKVA | carbamoyl-phosphate synthase | Unique | 302 |
| Human | NLMHISYEA | Argininosuccinate synthase | Unique | 303 |
| Human | LLDVPTAA | Ifn-g inducable protein 30 Kda | Unique | 304 |
| Human | FLKEPALNEA | Proteosome activating factor PA28 a-chain | Unique | 305 |
| Human | SLDQSVTHL | Intestinal alkaline phosphatase | Unique | 306 |
| Human | KIVVVTAGV | Lactate dehydrogenase B | Unique | 307 |
| Human | HLIEQDFPGM | HPAST | | 308 |
| Human | FGVEQDVDMV | Pyruvate kinase M2 | | 309 |

Viral Epitopes

| | | | | |
|---|---|---|---|---|
| WNV | RLDDDGNFQL | NS2b | Unique | 310 |
| WNV | ATWAENIQV | NS5 | Unique | 311 |
| WNV | SVGGVFTSV | Env | Unique | 312 |
| WNV | YTMDGEYRL | NS3 | Unique | 313 |
| WNV | SLTSINVQA | NS4b | Unique | 314 |
| WNV | SLFGQRIEN | NS4b | Unique | 315 |

The identification of novel, tumor-specific epitopes is a critical step in the development of T cell receptor mediated immunotherapeutics. Cells undergo a vast number of cellular changes during tumorigenesis, including genetic mutation, alterations in gene expression, and changes in protein processing. Some of these changes result in the secretion of biomarkers, such as the prostate specific antigen (PSA), 1 which serve as indicators of disease. Other cancer-related markers can be recognized on the outer surface of the cell by antibodies such as trastuzumab which binds the erbb2 growth factor receptor, specifically targeting HER-2/neu overexpressing tumors. Unfortunately, the vast majority of cellular changes associated with tumorigenesis are not secreted or found at the surface of cancerous cells; most cancer markers are intracellular in nature.

To convey intracellular health to the immune system, mammals utilize the major histocompatibility complex (MHC) class I molecule. Class I MHC molecules are nature's proteome scanning chip. The MHC I molecules gather many small peptides of intracellular origin, including the products of proteasomal processing and of defective translation, and carry these intracellular peptides to the cell surface. Intracellular peptides derived from proteins found in multiple compartments within the cell, and derived from proteins of many cellular functions, are sampled and presented at the cell surface by class I MHC. Immune cells including CD8+ cytotoxic T-lymphocytes (CTL) survey the peptides presented by class I MHC and target cells displaying cancer-specific peptides. Therefore, class I MHC presented peptides distinguish and promote the recognition of cancerous cells by the adaptive immune system.

Given that MHC class I distinguish cancerous cells from healthy cells, a number of studies have aimed to identify class I MHC presented cancer antigens. Because class I MHC molecules can be difficult to produce and purify, immune-based studies using CTL raised to autologous tumors have been utilized to identify cancer immune targets. Other immune-based methods have relied upon predictive algorithms and in vitro class I MHC peptide binding assays. Although these indirect approaches have identified putative tumor antigens, a direct proteomics based approach for identifying class I MHC tumor antigens is desirable. Proteomics-based methods are positioned to directly indicate the number of epitopes that uniquely decorate a cancer cell, serving to complement indirect immune-based methods for cancer epitope discovery.

Recognizing the protein production, isolation, and characterization challenges associated with the direct analysis of class I MHC proteome scanning chips, the inventors set out to obtain plentiful quantities of individual human class I MHC (HLA) from well-characterized cancer cell lines. Through expression of a secreted human class I MHC (sHLA) as described in the inventor's prior applications and discussed in detail herein above, the cell's own class I remain on the cell surface and only the transfected sHLA is harvested. Moreover, secretion of the human class I MHC molecule allows purification of the desired protein from tissue culture supernatants rather than isolating class I MHC from more complex detergent lysates.

Thus, the presently disclosed and claimed inventive concept(s) is directed to a method for producing and purifying plentiful class I from cancerous cell lines. Once the class I is harvested from cancerous cells, the sHLA is stripped of its peptide cargo, and comparative mass spectrometry is used to peruse cancer-specific class I peptide epitopes.

In the presently disclosed and claimed inventive concept(s), class I HLA A*0201 presented peptide epitopes of breast cancer cell lines are directly compared to those presented by a non-tumorigenic line. The class I HLA A*0201 allele was selected for its high frequency in the population. Tumorigenic cell lines, MDA-MB-231, MCF-7, BT-20, and the nontumorigenic cell line MCF10A were transfected with the sHLA-A*0201 construct. Peptides were purified from 25 mg of harvested sHLA-A*0201 produced by each cell line. Comparative mapping of thousands of sHLA-A*0201 derived peptides by mass spectrometry identified 5 previously uncharacterized epitopes unique to the tumorigenic cell lines (Table IV). Through characterization of protein expression, and by testing immune recognition of the epitopes, validation for these 5 breast cancer epitopes is provided herein. In addition, six peptides have been identified as up-regulated on breast cancer cells (Table V). The identification and characterization of these peptide epitopes are described in greater detail herein below.

TABLE IV
Peptide Epitopes Unique to Breast Cancer

| | | | SEQ ID | | | |
|---|---|---|---|---|---|---|
| | SEQ | | NO: for | | | |
| | ID | | source | Sequence | Presenting | Associated |
| SEQUENCE | NO: | SOURCE PROTEIN | protein | coverage | cell lines | Cancers |
| KIGEGTYGV | 316 | Cyclin Dependent | 327 | 9-17 | MCF-7, BT-20, | Breast, prostate, |
| | | Kinase 2 (CDK2) | | | MDA-MB-231 | lung, colon, |
| | | | | | | ovarian |
| ILDQKINEV | 317 | Ornithine | 328 | 23-31 | MCF-7, BT-20, | Breast, |
| | | Decarboxylase | | | MDA-MB-231 | pancreatic, |
| | | (ODC1) | | | | colon, liver, |
| | | | | | | lung, leukemia |
| GLNEEIARV | 318 | Kinetochore | 329 | 330-338 | MCF-7, BT-20, | Lung, prostate, |
| | | Associated 2 (KNTC2 | | | MDA-MB-231 | breast, ovarian, |
| | | or HEC1) | | | | lymphoma, |
| | | | | | | glioma |
| FLSELTQQL | 319 | Macrophage | 330 | 19-27 | MCF-7, MDA- | Breast, ovarian, |
| | | Migration Inhibitory | | | MB-231 | prostate, gastric, |
| | | Factor (MIF) | | | | colon, lung |
| ALMPVLNQV | 320 | Human mRNA | 331 | 214-222 | MCF-7, BT-20, | Unclear, RNA |
| | | Transport Regulator | | | MDA-MB-231 | processing could |
| | | (hMtr3p) | | | | lay a role in |
| | | | | | | many |

TABLE V
Peptide Epitopes Up-regulated in Breast Cancer

| | SEQ | | SEQ ID NO: | FOLD INCREASE | FOLD INCREASE | FOLD INCREASE |
|---|---|---|---|---|---|---|
| | ID | | for source | MCF-7 over | MDA-MB-231 | BT-20 over |
| SEQUENCE | NO: | SOURCE PROTEIN | protein | MCF10A | over MCF10A | MCF10A |
| KILDLETQL | 321 | ODF2/Cenexin | 332 | 9 | 1.4 | 7 |
| AQYEHDLEV | 322 | Ran GTPase | 333 | 34 | 2 | 8 |
| A | | | | | | |
| TLYEAVREV | 323 | RPL10a | 334 | None | 2.3 | 7.9 |
| SLLEKSLGL | 324 | P18 | 335 | 7.9 | 7.4 | None |
| SLFGGSVKL | 325 | PDCD6IP | 336 | 5 | 2.6 | 1.3 |
| SLFPGKLEV | 326 | Flightless Homolog | 337 | 5 | 2.2 | 2 |

Materials and Methods

Tissue Culture:

Tumorigenic breast cancer cell lines, MDA-MB-231, MCF-7, and BT-20 (ATCC), and a non-tumorigenic, immortalized cell line, MCF10A (ATCC), were cultured in DMEM/F12K (Caisson Laboratories, North Logan, Utah), 10% heat-inactivated FCS, and 100 units/mL Penn-Strep (Invitrogen, Carlsbad, Calif.). MCF10A culture medium was further supplemented with 20 ng/mL cholera toxin (Calbiochem, San Diego, Calif.), 0.5 μg/mL hydrocortisone (Sigma, St. Louis, Mo.), 10 μg/mL recombinant human insulin (Wisent, Saint-Jean-Baptiste de Rouville, Quebec, Canada), and 20 ng/mL recombinant human epidermal growth factor (Wisent).

Secreted HLA Production:

To produce secreted HLA molecules, α-chain cDNAs of the most common HLA allele, A*0201, were modified at the 3′ end by PCR mutagenesis to delete codons 5-7 encoding the transmembrane and cytoplasmic domains and to add a 30 base-pair tail encoding the 10 amino acid rat very low density lipoprotein receptor (VLDLr), SVVSTDDDLA, for purification purposes. 15 sHLA-VLDLr was cloned into the mammalian expression vector pcDNA3.1(−) Geneticin (Invitrogen) and then sequenced to ensure fidelity of each clone.

Cell Transfection:

Breast cancer cell lines (MCF-7, MDA-MB-231, and BT-20) and an immortal, non-tumorigenic breast epithelial cell line (MCF10A) were transfected with sHLA-A*0201 using the FuGENE® 6 Transfection Reagent kit (Roche Diagnostics Corp., Indianapolis, Ind.). Briefly, cells were grown in complete media to 80-85% confluency after which they were trypsinized and plated at 2×10⁵ cells/well in a 6-well tissue culture plate (Falcon, Becton Dickinson Labware, Franklin Lakes, N.J.) in 1 mL of serum-free media and grown overnight to reach 50-80% confluency before transfection. Cells were transfected by adding 100 μl of serum-free media containing 1 μg of DNA at 1:3 ratio DNA/FuGENE®. Plates were incubated after transfection for 24 h, and then received 1 mL of complete selective media containing the appropriate concentration of antibiotic.

VLDLr Capture ELISA:

Ninety-six well StarWell Maxisorp plates (Nalge Nunc International) were coated with 200 μl of mouse monoclonal anti-VLDLr (ATCC clone CRL-2197) antibody at 10.0 μg/mL in carbonate buffer, pH 9.0. Plates were incubated at 4° C. overnight and then blocked with 3% BSA in PBS for 2 h at room temperature. Standards were set in triplicates at 100, 80, 60, 40, 20, 10, 5, and 0 ng/mL (blank), using a known VLDLr-tagged sHLA molecule as a standard protein. Samples were incubated for 1 h at 37° C. Detection of sHLA molecules was performed using rabbit anti-β2 microglobulin (DAKO, Denmark) and HRP-donkey anti-rabbit (Jackson ImmunoResearch Laboratories) incubated 30 min each at room temperature. Following a 30 minute development with OPD (Sigma), the reaction was stopped with 3N H₂SO₄, and plates were read at 490 nm. Samples were quantified by comparing them to sHLA standards.

Subcloning and Large-Scale Production:

Transfected cells grown in selective antibiotic media were tested for production of sHLA molecules by ELISA. Positive wells were trypsinized and subcloned into 96-well plates (Falcon) by single cell sorting using the INFLUX™ Cell Sorter (Cytopeia, Seattle, Wash.). Individual wells with subcloned cells were tested for the production of sHLA, and positive wells were expanded for inoculation into bioreactors (Toray, Tokyo, Japan) in a CP2500 CELL PHARM® (Biovest International, Minneapolis, Minn.).

Peptide Purification.

Cell supernatants were passed over a SEPHAROSE® 4B precolumn to remove excess milk fat. Approximately 25 mg of A*0201VLDLr molecules from each cell line was purified over an affinity column composed of anti-VLDLr antibody coupled with CNBr activated SEPHAROSE® 4B (GE Healthcare, Piscataway, N.J.). sHLA molecules were then eluted in 0.2 N acetic acid, brought up to 10% acetic acid, and heated to 78° C. for 10 min. Peptides were separated from heavy and light chains by ultrafiltration in a stirred cell with a 3 kDa molecular weight cutoff cellulose membrane (Millipore, Bedford, Mass.). Each peptide batch was flash-frozen and lyophilized. The peptides were then reconstituted in 10% acetic acid. To be certain the peptides were derived from the HLA molecule of interest, 10% of the pooled peptides from each cell line was subjected to 14 rounds of Edman sequencing to confirm an A*0201 binding motif.

Reversed-Phase HPLC:

Peptides were reversed-phase HPLC fractionated with a 4 μm, 90 Å, 2×150 mm Jupiter Proteo C12 column (Phenomenex, Torrance, Calif.) on a Paradigm MG4 system (Michrom Bioresources, Auburn, Calif.) with a 1 mL stainless loop using an CH₃CN gradient as follows: 2% B for 11 min. (80 μL/min), 2-5% B in 0.02 min (80-160 μL/min), 5-40% B in 40 min (160 μL/min), and 40-80% B in 20 min (160 μL/min). Composition of solvents was as follows: solvent A, 98% H₂O, 2% CH₃CN, and 0.1% TFA (trifluoroacetic acid); and solvent B, 95% CH₃CN, 5% H20, and 0.08% TFA. Approximately 250 μg of total peptide was separated into 40 0.7-min fractions. UV absorption was monitored at 215 nm. Consecutive and identical peptide separations were performed for each peptide batch.

Mass Spectrometric Analysis:

Peptide fractions were concentrated to dryness by Speed-Vac and reconstituted in 20 μl of nanospray buffer composed of 50% methanol, 50% H₂O, and 0.5% acetic acid. Nanoelectrospray capillaries (Proxeon, Denmark) were loaded with 1 μl of each peptide fraction and infused at 1100 V on a Q-Star Elite quadrupole mass spectrometer with a TOF (time-of-flight) detector (Applied Biosystems, Foster City, Calif.) for 5 min. Triplicate ion maps were generated for each fraction in a mass range of 300-1200 amu. MS peak lists were generated with a threshold of 500 counts in Analyst QS 2.0 (ABI/MDS Sciex) and aligned for each fraction and each cell line using an internally generated Excel (Microsoft, 2003) script. Ions excluded from the alignment of tumorigenic versus nontumorigenic peak lists were selected as potentially unique. Spectra from corresponding fractions of each cell line were, also, aligned and visually assessed with a 20 amu window for the presence of unique ion peaks.

Unique peaks selected for further analysis were subjected to tandem mass spectrometry (MS/MS) and an amino acid sequence assigned to centroided, deisotoped data using the publicly available, Web-based MASCOT (Matrix Science Ltd., London, U.K.) and/or de novo sequencing. Search engine parameters were as follows: database NCBinr, human species, no enzyme, phosphorylation or sulfation allowed, and mass tolerance of 0.5 Da for both precursor and MS/MS data. Synthetic peptides, corresponding to each putative sequence, were produced and subjected to MS/MS under identical collision conditions as the naturally occurring peptide. The spectra produced were compared to confirm peptide sequence identity.

Synthetic Peptides.

Unmodified peptides were synthesized and purified by the Molecular Biology Resource Facility (The University of Oklahoma HSC, Oklahoma City, Okla.). Purity was determined to be greater than 95%. The composition was ascertained by mass spectrometric analysis.

IC₅₀.

The binding affinity of the individual peptides for the HLA A*0201 was determined using a competitive binding, fluorescence polarization based assay, PolyTest (Pure Protein, LLC, Oklahoma City, Okla.). In brief, the peptide of interest is incubated with a FITC (fluorescein isothiocyanate) labeled reference peptide and soluble HLA class I heavy and light chains. Displacement of the reference peptide by the competitor results in increased rotational mobility of the labeled peptide and decreased polarization. The IC₅₀ is determined by the concentration of the competing peptide required to inhibit 50% binding of the reference peptide to the HLA molecule. For this assay, an IC50<5000 nM is considered high affinity.

Western Blot:

Cell lysates were generated from cell lines using the RIPA buffer and HALT Protease Inhibitor Cocktail Kits (Pierce) according to manufacturer's instructions. SDSPAGE was performed with 10 μg of total protein loaded onto 4-12% NuPAGE Bis-Tris precast gels in MOPS buffer (Invitrogen). Protein was blotted on PVDF membrane and probed with mouse monoclonal anti-ODC1, anti-Cdk2, anti-KNTC2 (Novus Biologicals), or rabbit polyclonal anti-EXOSC6 (Abcam). Proteins were detected using HRP-conjugated Donkey anti-mouse IgG or Donkey anti-rabbit IgG (Jackson Immunoresearch) and SUPERSIGNAL® Chemiluminescent Substrate (Pierce). All blots were stripped with Restore Western Blot Stripping Buffer (Pierce) and reprobed with mouse monoclonal anti-βactin (Sigma).

MIF protein was immunoprecipitated from 1 mL of 3 day confluent cell culture supernatants using mouse monoclonal anti-MIF (Novus Biologicals) coated Protein G SEPHAROSE® beads (GE Healthcare). Electrophoresis and detection were performed as above.

Research Participants:

Subjects, with or without a prior history of breast cancer (Ductal Carcinoma In Situ or Infiltrating Ductal Carcinoma), were recruited according to OUHSC Institutional Review Board approved protocol number 13571. Participant HLA type was determined by Sequence Based Typing and confirmed by flow cytometry of PBMC stained with FITC labeled BB7.2 anti-HLA-A*0201 antibody. Nine HLAA*0201 positive subjects were identified from each group. Forty milliliters of whole blood was collected from each participant and processed for Peripheral Blood Mononuclear Cells (PBMC).

Tetramer Staining:

HLA-A*0201 positive PBMC were separated by Lymphoprep gradient (Axis-Shield). PBMC were resuspended in Cell Staining Buffer (Biolegend), and 1×10⁶ cells were stained for 30 min at 4° C. in a 1:100 dilution of Allophycocyanin (APC) labeled MHCI tetramer (NIH Tetramer Facility or Protein Chemistry Core, Baylor College of Medicine), FITC anti-CD8R (Biolegend), and PerCP-Cy5.5 anti-CD3 (Becton Dickenson). PBMC were washed, fixed in 1% paraformaldehyde (PFA) in phosphate buffered saline (PBS), and analyzed on a FACScaliber (Becton Dickenson).

ELISPOT:

Fresh PBMC were stimulated for 1 week with 2 μg of peptide in RPMI 1640 with 10% fetal bovine serum. Recombinant human IL-2 (Invitrogen) was added at 0.3 ng/mL on days 3, 5, and 7. PBMC were rested 48 h prior to ELISPOT. A total of 1×10⁵ cells/well was plated on antihuman IFN-γ coated plates (SeraCare) with 2 μg of peptide or Phytohemagglutinin-H (Sigma) as a positive control. Plates were developed according to kit instructions. Plates were read on an ImmunoSpot plate reader and analyzed using ImmunoSpot v. Four (Cellular Technology, Ltd.).

Results

MS Comparative Analysis. Peptides were eluted from 25 mg of purified sHLA A*0201 harvested from three tumorigenic breast epithelial cell lines (MCF-7, MDA-MB-231, and BT-20) and the non-tumorigenic breast epithelial line (MCF10A). The A*0201 peptide binding motif was confirmed by Edman sequencing 10% of pooled peptides from each cell line (data not shown). The peptide batches were consecutively fractionated by RP-HPLC. During mass spectrometric analysis, alignment of corresponding fractions was confirmed by the presence of identical peptides across the panel.

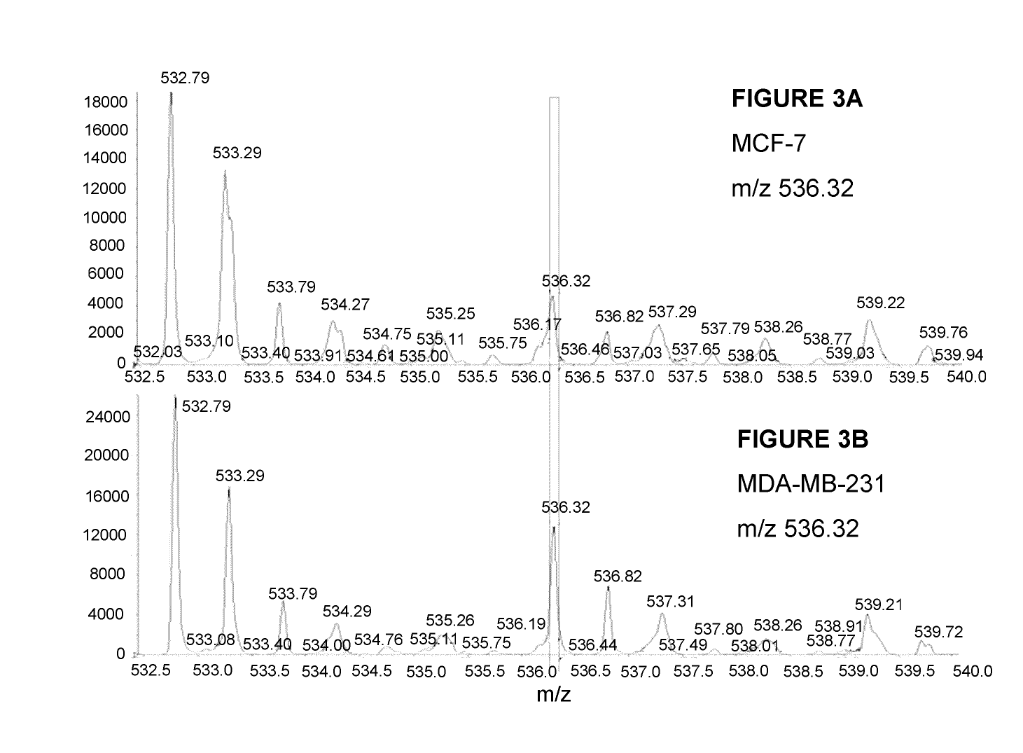

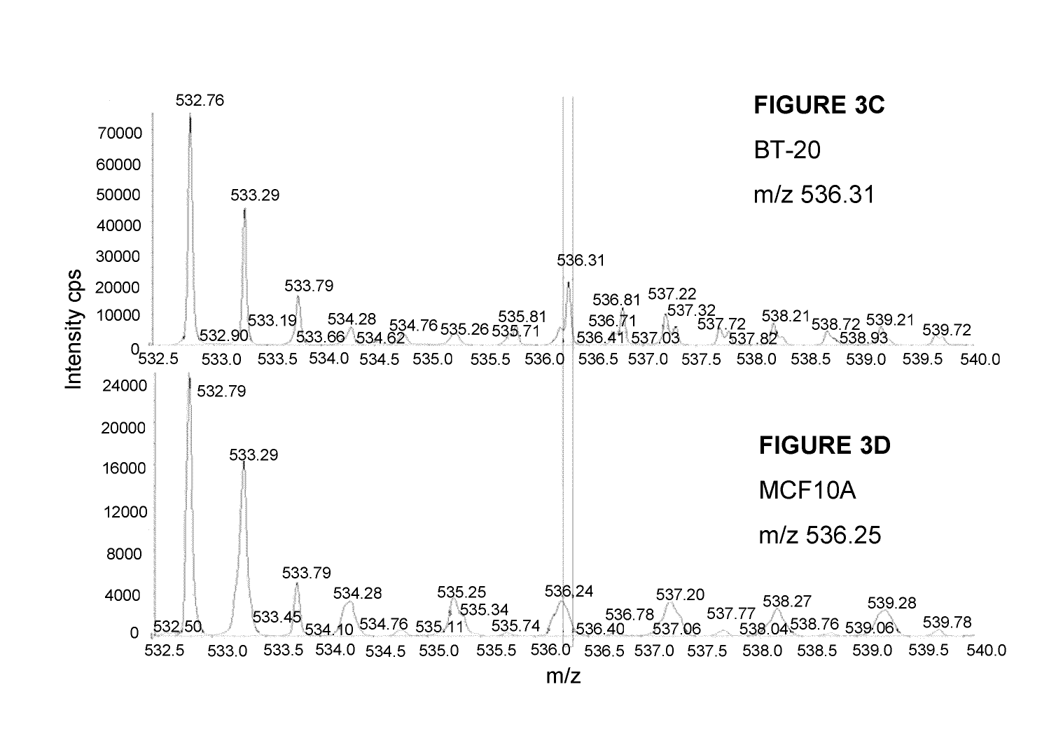

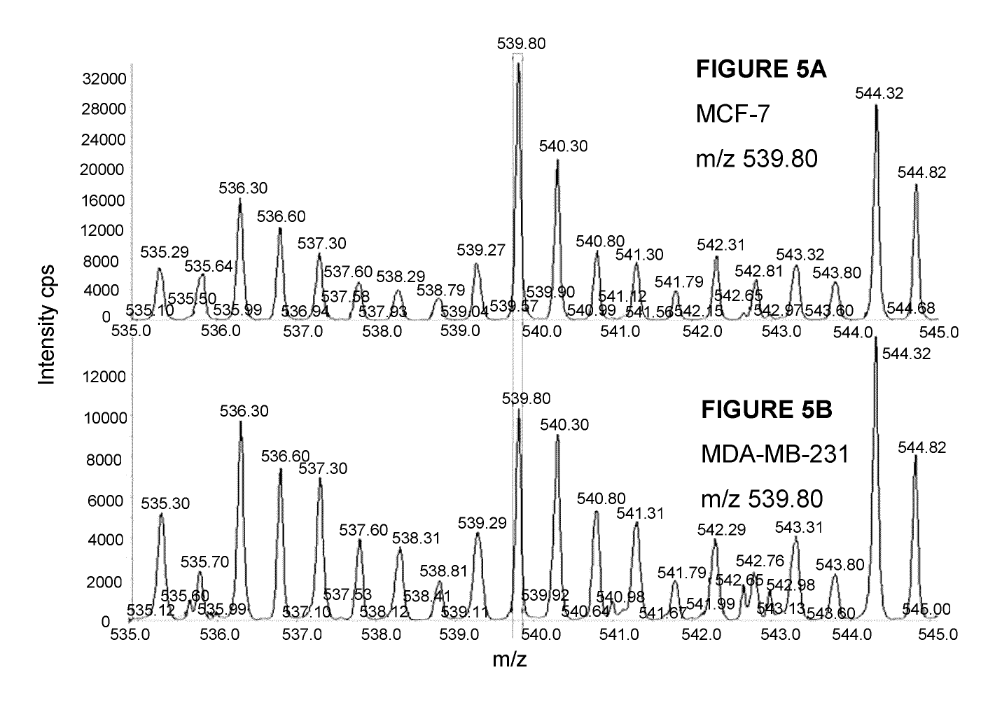

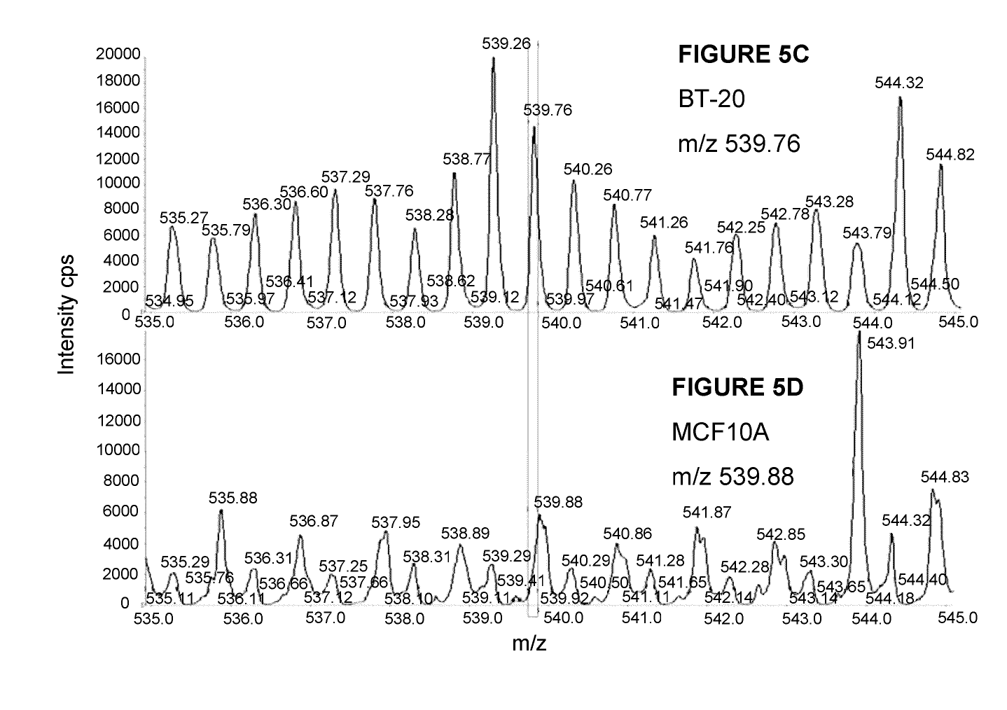

Triplicate MS ion maps were generated from each of 40 peptide containing fractions for each cell line. Peak lists from tumorigenic and non-tumorigenic peptide batches were aligned from corresponding fractions and excluded peaks were treated as potentially unique to the tumorigenic lines. Additionally, spectra from corresponding fractions were examined visually to identify or confirm the presence of ion peaks that could represent peptides unique to the tumorigenic lines. Five ion peaks were identified as being shared among tumorigenic cell lines and absent from the MCF10A. These peaks were +2 ions at 536.32 m/z (FIG. 3), 539.8 m/z (FIG. 5), 492.77 m/z, 462.24 m/z, and 500.77 m/z (data not shown).

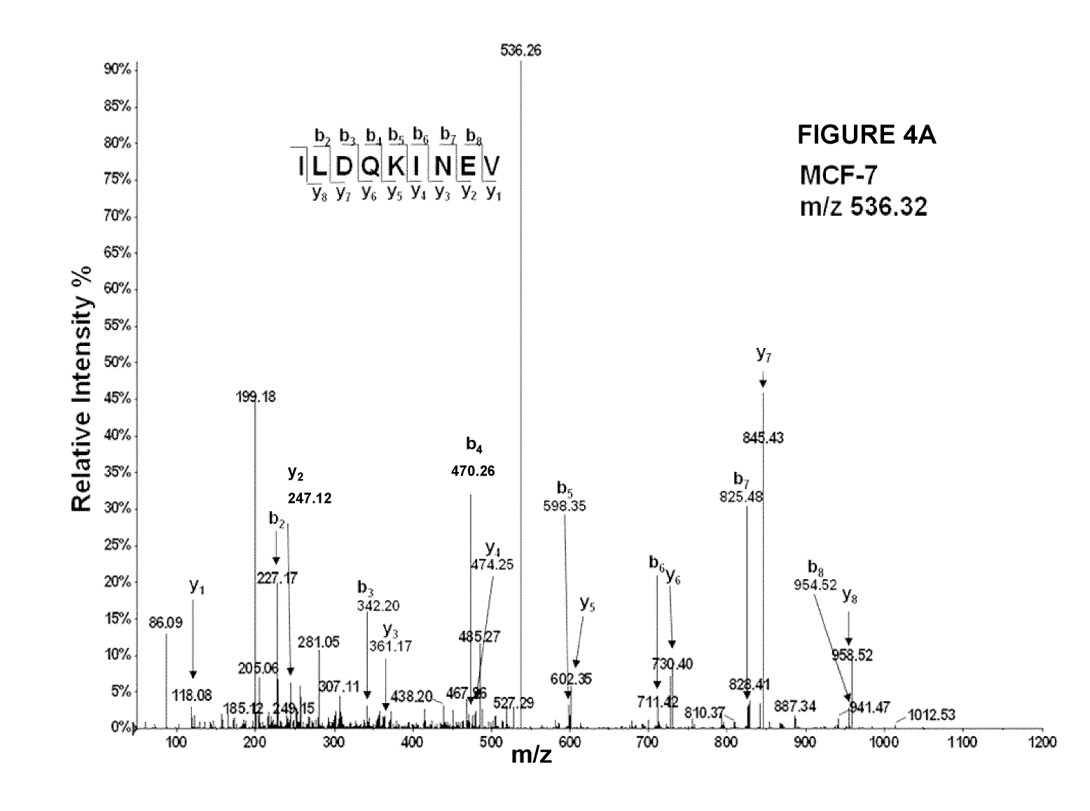

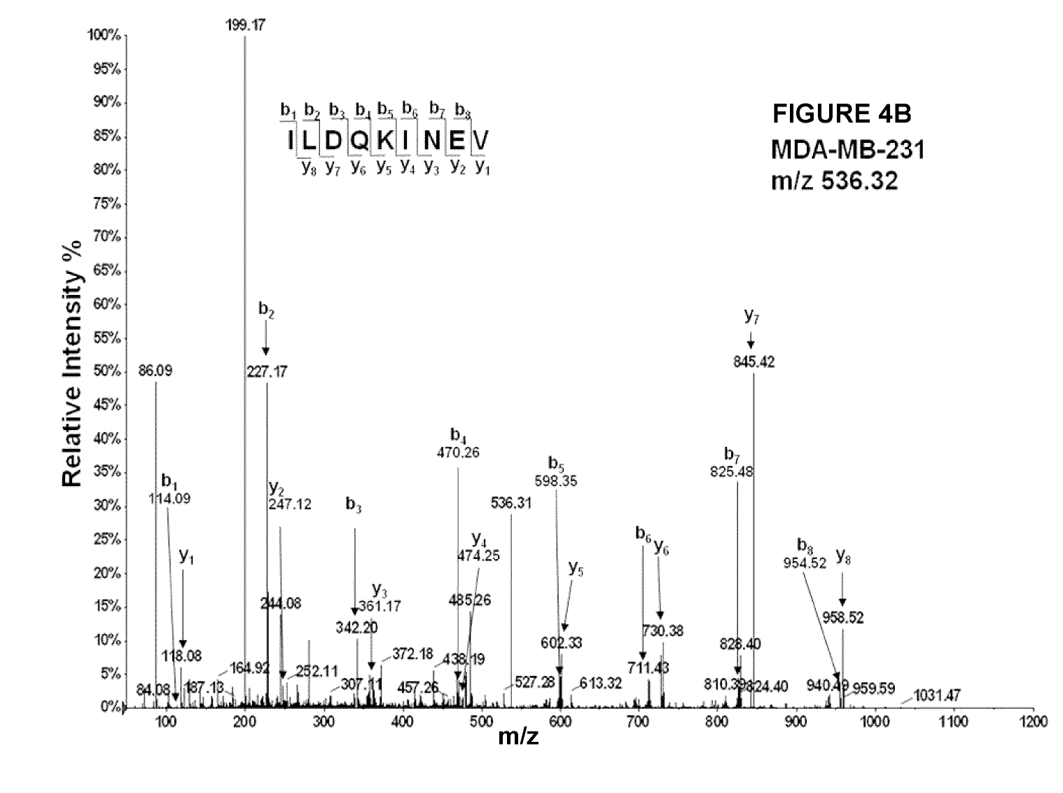

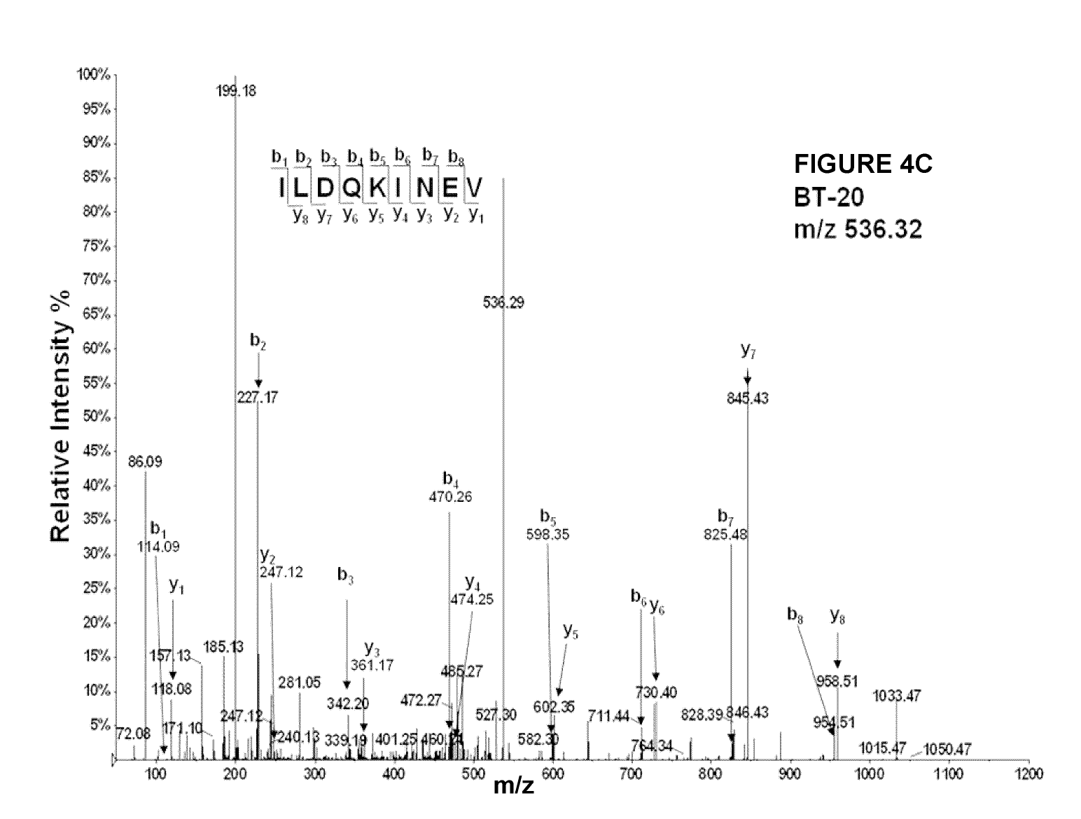

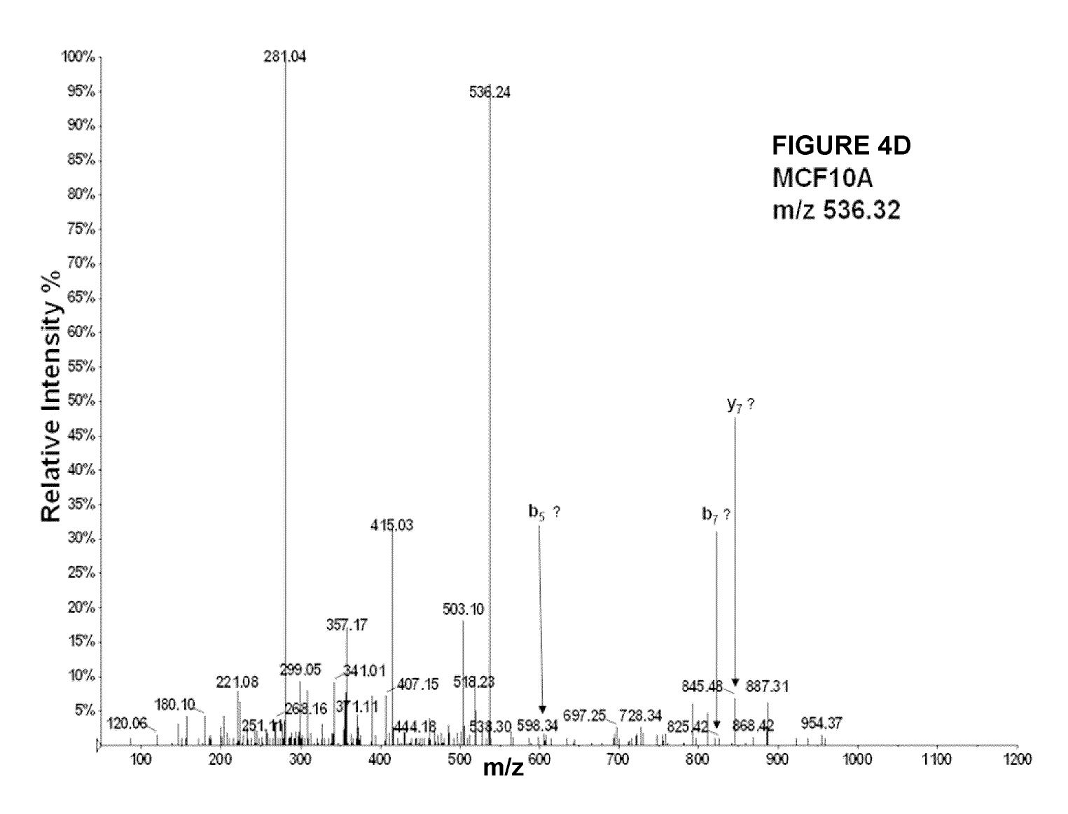

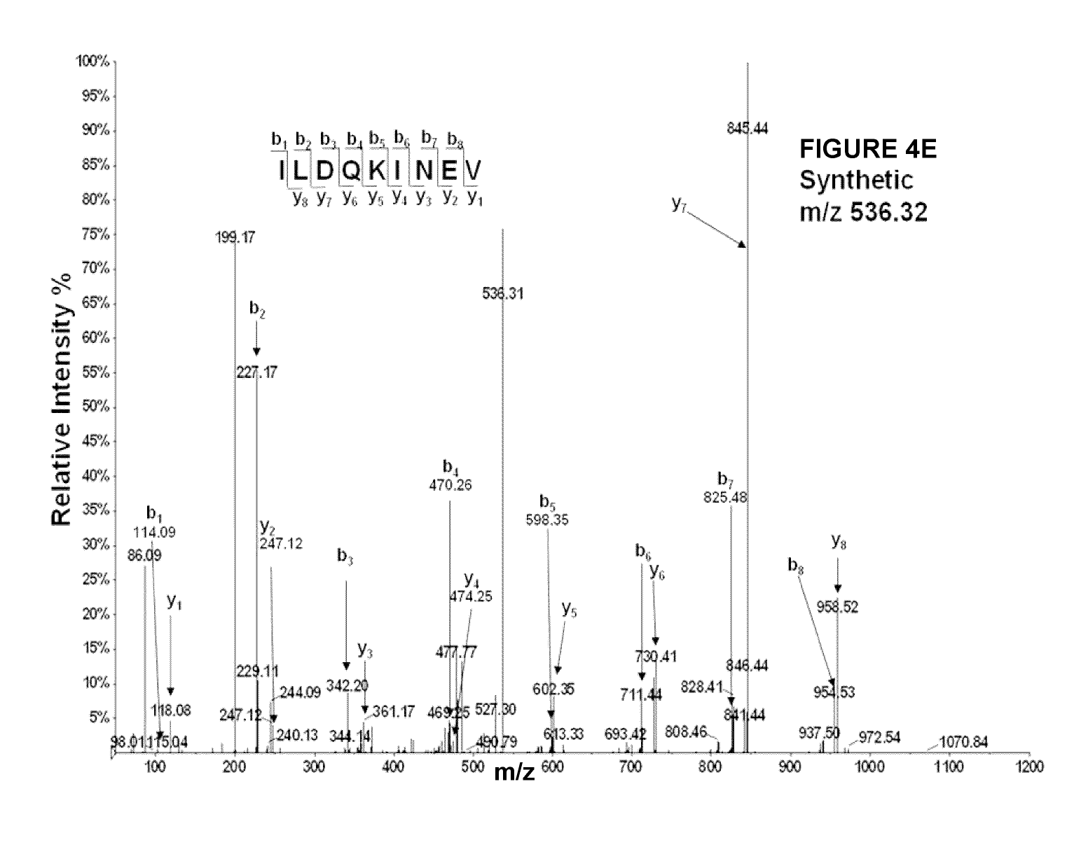

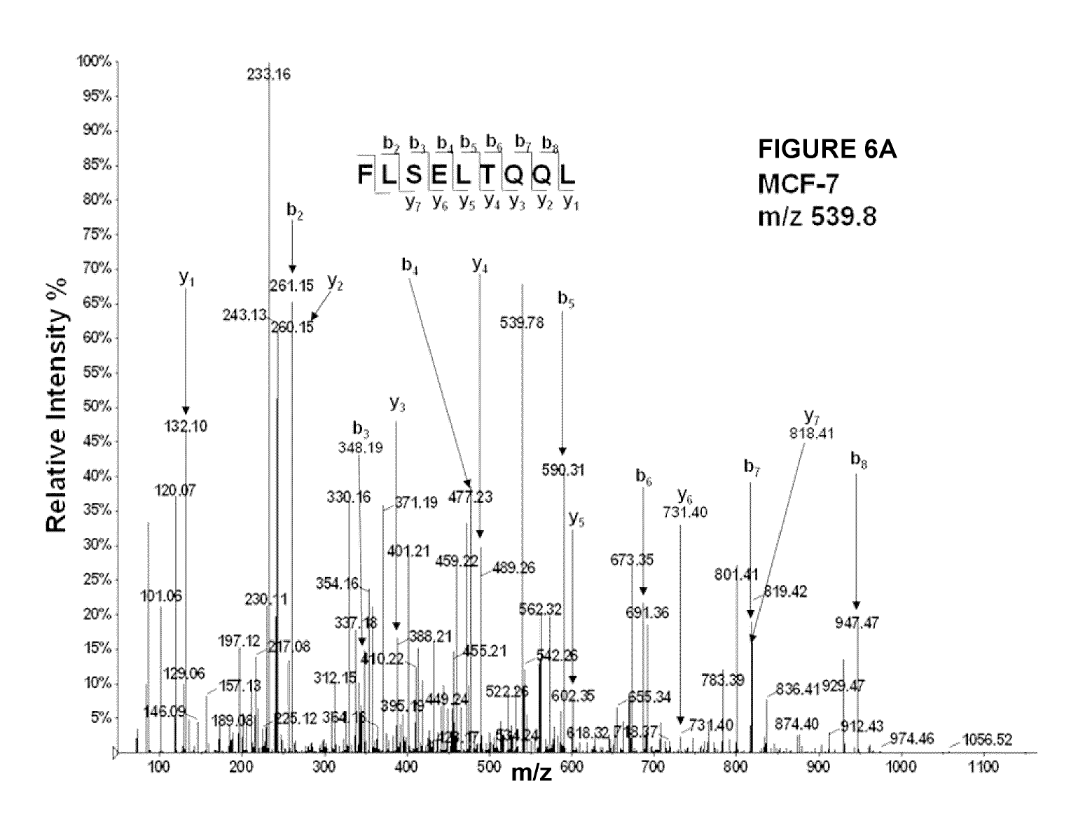

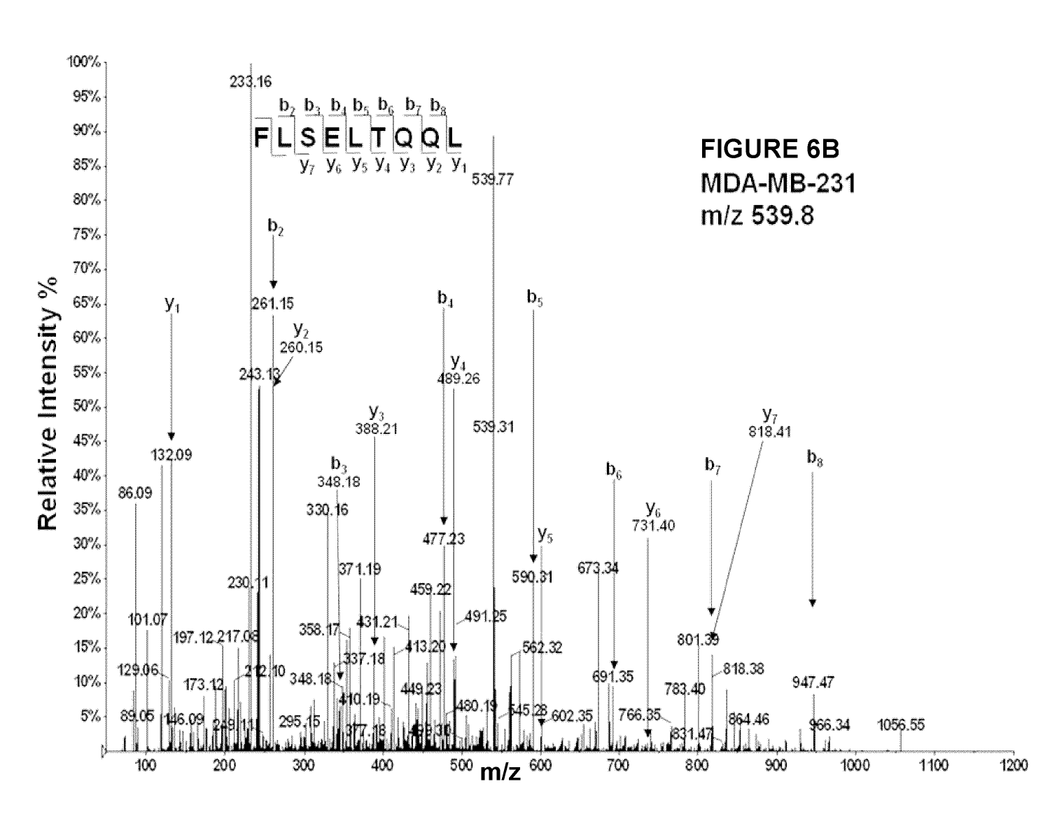

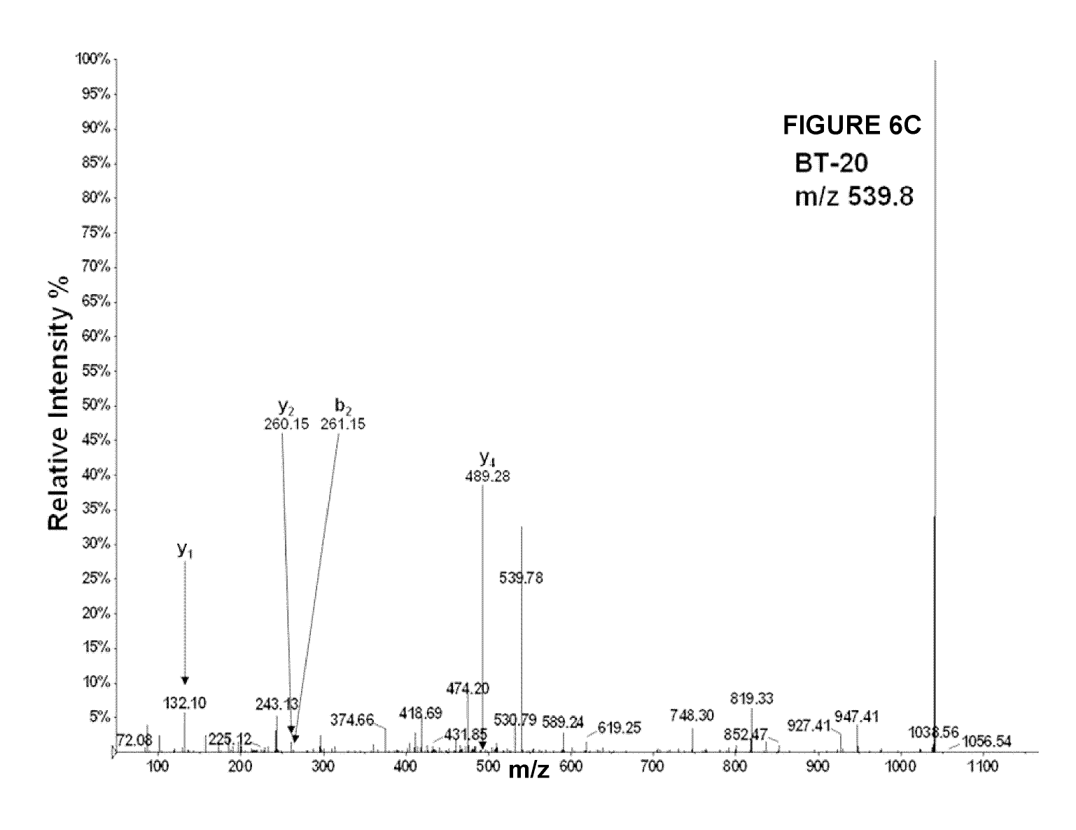

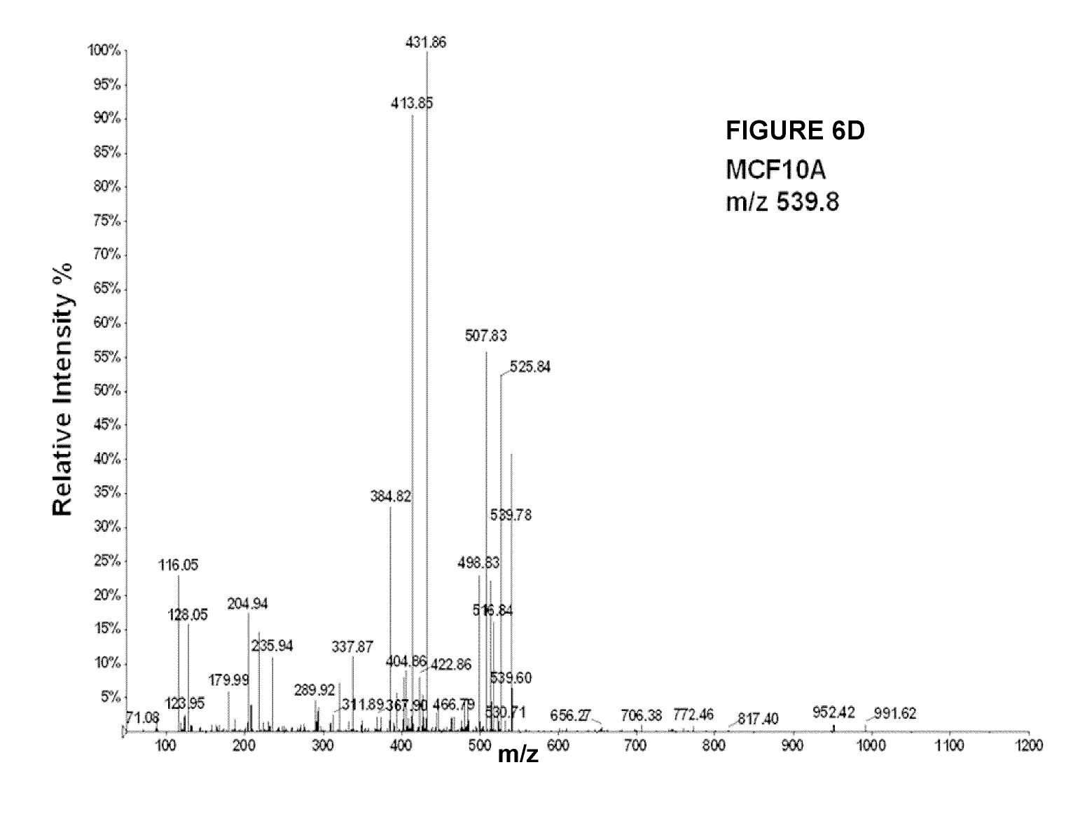

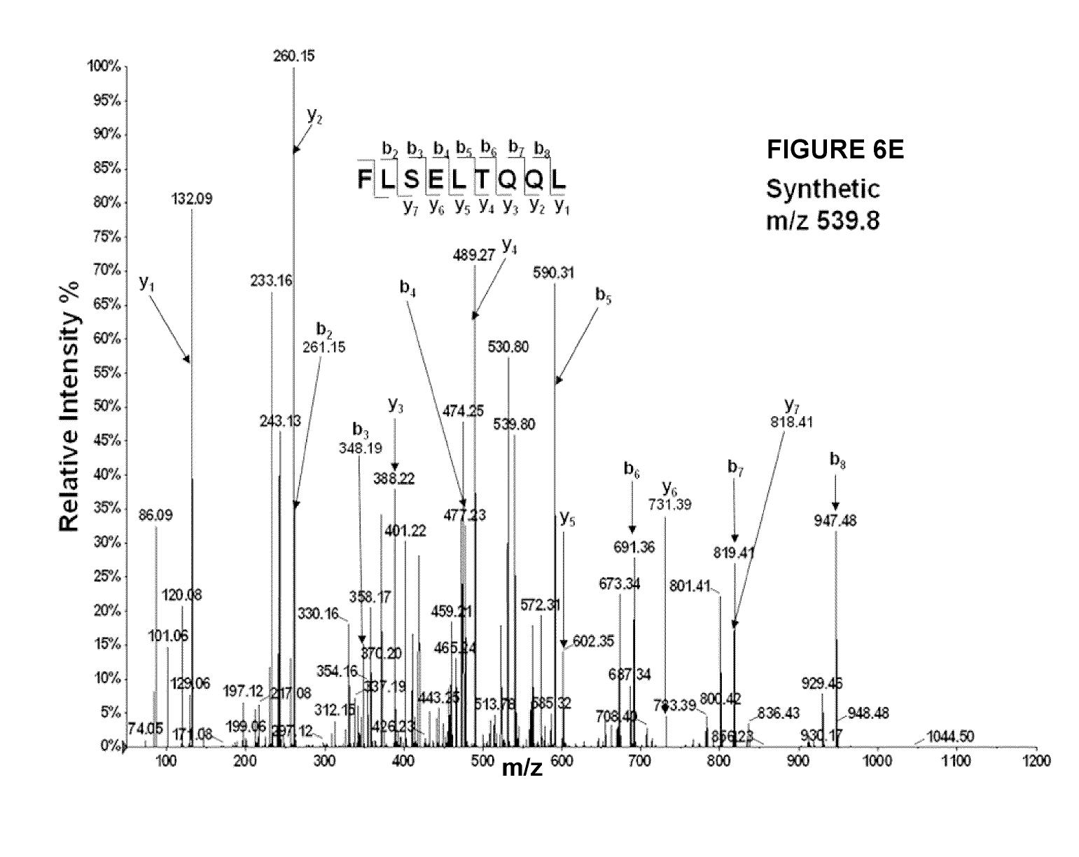

Identification of Peptides Uniquely Presented by HLA-A*0201. Potentially unique ions were selected for MS/MS fragmentation and a sequence assigned using MASCOT. Examples of the product ion spectra are shown in FIGS. 4 and 6. The peptide sequence of ion peak 536.32 m/z was ILDQKINEV (SEQ ID NO:317), which corresponds to positions 23-31 of Ornithine Decarboxylase (ODC1). The sequence of ion peak 539.8 m/z was FLSELTQQL (SEQ ID NO:319), which corresponds to positions 19-27 of Macrophage Migration Inhibitory Factor (MIF). The sequence of ion peak 492.77 m/z was ALMPVLNQV (SEQ ID NO:320), which corresponds to positions 214-222 of Exosome Component 6 (EXOSC6). The sequence of ion peak 462.24 m/z was KIGEGTYGV (SEQ ID NO:316), which corresponds to positions 9-17 of Cyclin Dependent Kinase 2 (Cdk2). The sequence of ion peak 500.77 m/z was GLNEEIARV (SEQ ID NO:318), which corresponds to positions 330-338 of Kinetochore Associated 2 (KNTC2 or HEC1). Table IV provides a more detailed description of the peptides. Most of the source proteins of these peptides, such as ODC, MIF, KNTC2, and Cdk2, have well defined roles in the development and progression of many cancers which are addressed in the Discussion. The role of EXOSC6 in tumor development is unclear, but putative associations are possible.

Over 150 peptides presented by the HLA A*0201 from the 3 tumorigenic cell lines and the non-tumorigenic line were sequenced. Most of these peptides were shared by all 4 cell lines and were used to ensure proper alignment of the fractions, including the bona fide CTL epitope, GLIEKNIEL from DNA methyl transferase 1 (SEQ ID NO:338; Berg et al., 2004). A few peptides were unique to an individual cell line, such as LLQEVEHQL (SEQ ID NO:339) from the E3 ubiquitin ligase TRIM37 found only in the MCF-7 peptide pool. The peptides corresponding to ODC1, Cdk2, EXOSC6, and KNTC2 were presented by the HLA A*0201 of all three tumorigenic lines and missing from the MCF10A pool. The MIF peptide was only identified in the MCF-7 and MDA-MB-231 batches. Although seemingly absent from the BT-20 batch, the MIF peptide may in fact be present at low concentration and therefore masked by the isotope of the overlapping peptide at 539.26 m/z. Further separation would be required to confirm or deny the possibility. However, the relevance of MIF to tumor development, progression, and metastasis makes it an attractive target even if its presentation is limited to a subset of tumors.

Validation of Unique Peptide Ligands. Three fractions preceding and following the fraction of interest were examined to confirm the unique nature of these peptides. Synthetic peptides were produced and subjected to MS/MS under identical collision conditions, and spectra were compared with native peptide to confirm peptide sequences (see, for example, FIGS. 4 and 6). The 5 peptides identified were determined to have high affinity for the HLA A*0201 using a competitive binding, fluorescence polarization based assay.

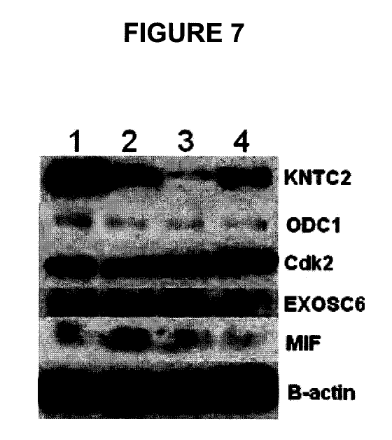

Confirmation of Protein Expression. Western blotting was performed to confirm expression of the peptide source proteins by the different cell lines (FIG. 7). A 75 kDa band was detected by the anti-KNTC2 antibody at various levels in lysates from all four cell lines. A 32 kDa band was detected by the anti-Cdk2 antibody at almost identical levels in all four cell lines. A 28 kDa band was present in all four lysates, corresponding to EXOSC6. The secreted protein MIF was not detected in any lysates but could be immunoprecipitated from tissue culture supernatants. In FIG. 7, immunoprecipitated MIF is visible as a band at 12 kDa. Interestingly, the BT20 cell line produced the highest level of MIF but did not present MIF peptide on the HLA molecule. ODC1 was faintly detected as a 53 kDa band in lysates from MDA-MB-231, BT-20, MCF-7, and MCF10A cell lysates (FIG. 5). The source proteins were expressed by all cell lines, suggesting a disconnection between expression and class I presentation.

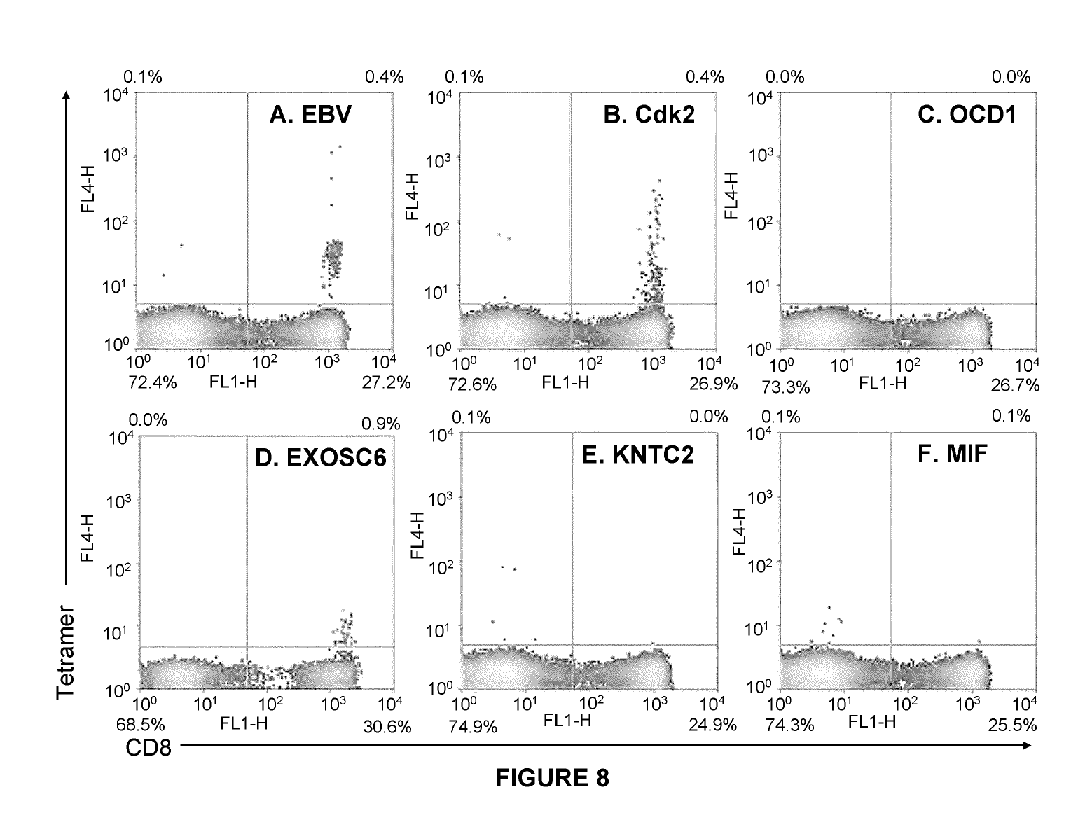

Immune Recognition of Breast Cancer Associated Peptides. Fresh PBMC from 11 HLA-A*0201 subjects with or without a history of breast cancer were stained with HLA-A*0201 tetramers comprised of ODC1, Cdk2, KNTC2, EXOSC6, MIF, and the Epstein-Barr Virus BMLF1 peptides. EBV BMLF1 represents a positive control. Subject 6, with a positive history of breast cancer, displayed CD8+ recognition of the Cdk2, EXOSC6, EBV control tetramers (FIG. 8).

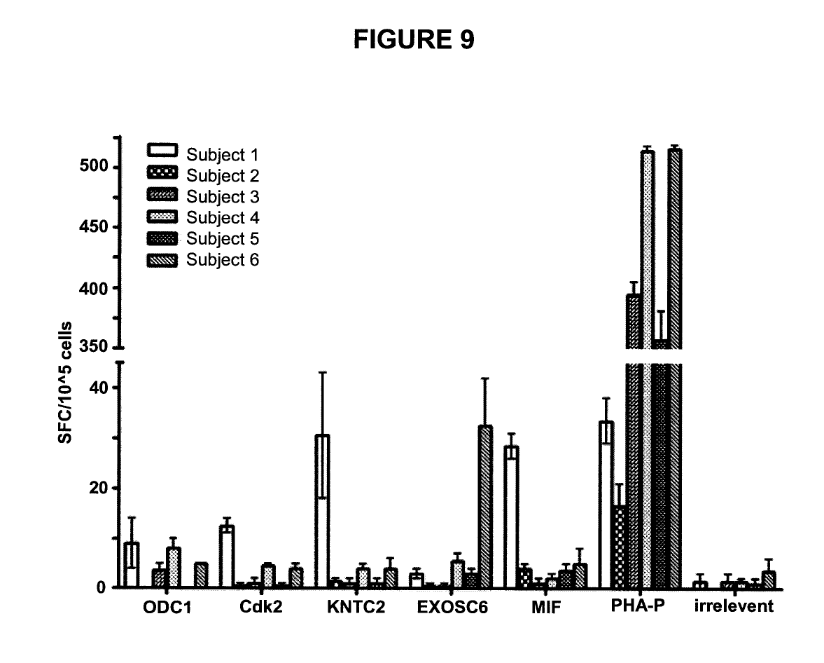

To test for functional immune recognition of the newly discovered breast cancer epitopes, PBMC from 6 subjects were stimulated in vitro for 1 week prior to IFN-γ ELISPOT testing. Subjects 1, 3, 4, 5, and 6 had a positive history of breast cancer, while subject 2 had no history of breast cancer. Subject 1 produced a relatively robust IFN-γ response to KNTC2 and MIF (FIG. 9). Subject 6 produced a robust response to EXOSC6, which recapitulates the tetramer staining (FIG. 8). Interestingly, subject 6 PBMC did not produce IFN-γ in response to Cdk2 (FIG. 9) despite staining of CD8+ cells with Cdk2 tetramers (FIG. 8). Further phenotypic characterization of these Cdk2 tetramer+/IFN-γ ELISPOT-cells is warranted.

To determine whether an immune response is generated to the peptides identified as specifically presented by tumorigenic cell lines, peripheral blood mononuclear cells were collected from a total of 6 HLA_A*0201+ control subjects and 7 HLA_A*0201+ breast cancer survivors for testing. Cells were tested for recognition of the identified peptides using 4 common immunologic assays: tetramer staining, Interferon Gamma (IFN-γ) ELISPOT, Intracellular cytokine staining for IFN-γ, and CD107a cytotoxicity staining. Three breast cancer

TABLE VI
SUMMARY OF IMMUNE RESPONSES TO IDENTIFIED
BREAST CANCER PEPTIDE EPITOPES

| | | Tetramer | IFN-γ | Intracellular | CD107a |
|---|---|---|---|---|---|
| Method: | | Stain | ELISpot | Cytokine IFN-γ | Cytotoxicity |
| Peptide | SEQ ID NO: | Patient #004 | | | |
| ILDQKINEV | 317 | − | ++ | ++ | − |
| KIGEGTYGV | 316 | − | − | + | − |
| GLNEEIARV | 318 | − | − | − | − |
| ALMPVLNQV | 320 | − | + | + | − |
| FLSELTQQL | 319 | − | + | − | − |
| Peptide | SEQ ID NO: | Patient #053 | | | |
| ILDQKINEV | 317 | − | − | − | − |
| KIGEGTYGV | 316 | ++ | − | + | + |
| GLNEEIARV | 318 | + | − | + | + |
| ALMPVLNQV | 320 | ++ | ++ | + | ++ |
| FLSELTQQL | 319 | − | − | ++ | ++ |
| Peptide | SEQ ID NO: | Patient #054 | | | |
| ILDQKINEV | 317 | + | ++ | ++ | + |
| KIGEGTYGV | 316 | ++ | + | ++ | ++ |
| GLNEEIARV | 318 | − | − | + | + |
| ALMPVLNQV | 320 | ++ | − | + | − |
| FLSELTQQL | 319 | + | ++ | − | + |

survivors had activated, memory, CD8+, cytotoxic T lymphocytes that recognized multiple identified peptides. These lymphocytes were capable of killing T2 cells pulsed with the specific peptide and were, additionally, capable of killing the MCF-7 cell line which naturally presents these peptides. A summary of these results are found in Table VI.

Discussion

Given that class I HLA molecules decorate the cell surface with intracellular peptide epitopes, a number of indirect methods have been used to identify HLA associated tumor rejection antigens. Purification of HLA associated peptides from cell lysates has also revealed a small number to tumor antigens. However, innate difficulties associated with protein production, purification, and peptide yield make direct analysis with the HLA molecule and its peptide ligands problematic when working from detergent cell lysates. Recognizing the power of HLA class I to distinguish cancerous cells, the inventor developed a method for producing plentiful class I without detergent lysis. The class I peptide cargo was then isolated, and cancerous and noncancerous peptide epitopes were compared by mass spectroscopy. This approach provides a direct proteomics view of the peptide epitopes that decorate well-characterized breast cancer cell lines. In the presently disclosed and claimed inventive concept(s), at least five epitopes were identified that represent intuitive targets for breast cancer therapies as well as therapies directed to a variety of other tumor types. Below, the characteristics of the parental proteins and their relevance to cancer are discussed.

ODC. Ornithine decarboxylase is an enzyme required for polyamine synthesis. It catalyzes the initial conversion of L-ornithine to putrescine, which is subsequently converted to spermidine and then spermine by S-adenosylmethionine decarboxylase. These polyamines act as organic cations and are required for cellular proliferation, differentiation, and transformation. The ODC gene promoter is a target of the oncogene myc, Ras activation pathways and estrogen mediated activation through cAMP/PKA. Through mRNA microarray, Western blot, enzymatic activity, and immunohistochemistry, a great deal is known about the expression patterns of ODC in primary tissues and numerous cell lines, including those examined herein. Expression of ODC tends to be very low in terminally differentiated tissues but very high and even prognostic in numerous tumors, including but not limited to breast, lung, and prostate cancer. Polyamine analogues and ODC-targeted siRNA have been shown to induce cell cycle arrest and inhibit proliferation and tumor invasiveness. ODC protein has a high turnover rate mediated by ubiquitin-dependent and -independent mechanisms that target the protein to the proteasome, the source of MHC class I peptides. The ILDQKINEV (SEQ ID NO:317) peptide was previously eluted from the TAP deficient, HLA-A*0201 positive T2 cell line, a Tx B cell hybridoma, transfected with TAP1 and either the TAP2*B or TAP2*Bky2 alleles (Kageyama et al., 2004). Overexpression coupled with a high rate of proteasomal degradation make ODC a prime target for HLA presentation on cancer cells. Although no immune recognition was detected by the cohort of study participants, the possibility of cellular recognition with immunization or targeting of the peptide/HLA complex by T cell Receptor mimic antibodies (TCRm) is recognized (see for example, US Patent Publication Nos. 2006/0034850, published Feb. 16, 2006; and 2007/00992530, published Apr. 26, 2007; the entire contents of both of which are hereby expressly incorporated herein by reference).

MIF. Macrophage Migration Inhibitory Factor is a multifunctional cytokine produced by a variety of normal and tumor cell types. MIF protein suppresses T and NK cell activity and may play at least a contributory role in maintaining immune privilege in the eye and the maternal/fetal interface. MIF binds the cell surface CD74 receptor which signals through the MAPK pathway to activate proliferation via cyclin D1, AP-1 mediated up-regulation of pro-inflammatory cytokines, and cellular adhesion molecules which play a role in tumor metastasis. In addition, MIF inhibits apoptosis by activation of Akt and by suppression of p53 mediated via E2F pathway modulation and COX-2 activation. MIF expression is important for neo-angiogenesis, proliferation, and invasiveness of neuroblastoma, hepatocellular, breast, prostate, and gastric carcinomas. Although the presentation of MIF-derived peptides by the HLA molecule has not been previously described, MIF protein expression by the four cell lines has been demonstrated here and by others. Protein expression data therefore suggests that MIF peptide presentation is plausible, while an IFN-γ response to MIF demonstrates that the immune system can respond to class I HLA presented MIF peptides.

EXOSC6. Exosome Component 6 is one of 11-16 exonucleases that make up the human exosome and is the homologue of the yeast mRNA Transport Regulator 3 (Mtr3p). The mammalian cell contains nuclear exosomes, responsible for processing of the 5.8 S rRNA, small nuclear RNAs (snRNA), and small nucleolar RNAs (snoRNA), and cytoplasmic exosomes, responsible for the 3′-5′ degradation of mRNAs containing AU rich elements (ARES) within the 3′ UTR. ARE containing mRNAs, generally, have short half-lives of 5-30 min including a large number of tumor associated transcripts, such as c-myc, cyclin D1, and COX-2. Degradation is mediated by the ARE binding protein, AUF1. These transcripts are often stabilized in cancer by the overexpression of Hu family ARE binding proteins, which displace AUF1. A direct role for the exosome and its components in tumorigenesis is unclear. However, deregulation of RNA turnover can result in cellular transformation, so a putative role for the exosome in tumorigenesis is reasonable. Interestingly, several exosome components are autoantigenic with a high degree of association to HLA-DR3. Patients suffering from poly-myositis and scleroderma, for which the complex was originally named (PM/Scl), have high titer antibodies primarily to the PM/Scl 100 component. In FIG. 7, expression of the EXOSC6 protein is demonstrated in all 4 cell lines, such that cancer-specific cellular mechanisms affecting protein decay may be involved in the class I HLA presentation of this peptide. The EXOSC6 peptide, ALMPVLNQV (SEQ ID NO:320), identified here as uniquely expressed by the tumorigenic breast epithelial lines, was previously eluted from an ovarian carcinoma line, UCI-107 (Milner et al., 2006). This peptide may be presented on a range of tumor cells. With ELISPOT and tetramer staining confirming immune recognition, the EXOSC6 epitope may act to distinguish a number of cancerous cells for immune surveillance mechanisms.

Cdk2. Cyclin Dependent Kinase 2 is a serine/threonine kinase that complexes with cyclin E to mediate the terminal phosphorylation and inactivation of retinoblastoma protein. This in turn releases sequestered E2F transcription factors, allowing transcription of genes required for G1 to S phase transition. Induction of cyclin D1, Cdk 4/6, cyclin E, and Cdk2 can be accomplished in tumors via loss of INK4 Cdk inhibitors, such as p16, or stimulation by mitogens, such as insulin, insulin-like growth factor I, and estrogen. Increased expression and increased activation of cyclin E and Cdk2 are reported in numerous tumor types, including breast, prostate, ovarian, and lung carcinomas. The presentation of Cdk2 derived peptides by the HLA molecule has not been previously described. However, protein expression in all 4 cell lines characterized in this study has been demonstrated, making peptide availability to the HLA molecule probable. Degradation of cell cycle associated proteins is tightly regulated such that disregulation of Cdk2 processing in tumorigenic cells may well result in presentation by an HLA molecule. In FIG. 8, it is shown that CD8+ cells in a breast cancer patient recognize the Cdk2 tetramer, although the lack of an IFN-γ response in the presence of CD8+ tetramer staining suggests a population of regulatory cells. How the immune response views CdK2 needs further exploration.

KNTC2. Kinetochore Associated 2, also known as Highly Expressed in Cancer (HEC1), is required for proper chromosome segregation. KNTC2 and Nuf 2 form a contact point for microtubule attachment to the kinetochore complex during mitotic spindle assembly55 and may act as a spindle checkpoint. KNTC2 binds the C-terminus of retinoblastoma protein (Rb) and interacts with the 26S proteasome subunit MSS1 to inhibit degradation of mitotic cyclins during M phase. In the absence of Rb, abnormal expression of spindle checkpoint proteins can lead to uncoupling of mitosis from the cell cycle and aneuploidy. KNTC2 is expressed only in actively proliferating cells and was identified as 1 of 11 genes corresponding to a “Death from Cancer” expression signature. KNTC2 is overexpressed in numerous tumor types, including prostate, breast, lung, ovarian, lymphoma, mesothelioma, medulloblastoma, glioma, and acute myeloid leukemia. Presentation of KNTC2 derived peptides by HLA molecules has not been previously described, but the confirmed expression of KNTC2 by all cell lines is consistent with epitope presentation (FIG. 7). Again, loss of control over a highly regulated system may explain the differential presentation of KNTC2 peptide by the tumorigenic and nontumorigenic cell lines. An IFN-γ response to the KNTC2 peptide indicates this protein is immunogenic.

Thus, in accordance with the presently disclosed and claimed inventive concept(s), there has been provided a method of epitope discovery and comparative ligand mapping that includes methodology for producing and manipulating Class I and Class II MHC molecules from gDNA as well as methodology for directly discovering epitopes unique to infected or tumor cells that fully satisfies the objectives and advantages set forth herein above. Although the inventive concept(s) has been described in conjunction with the specific drawings, experimentation, results and language set forth herein above, it is evident that many alternatives, modifications, and variations will be apparent to those skilled in the art. Accordingly, it is intended to embrace all such alternatives, modifications and variations that fall within the spirit and broad scope of the inventive concept(s).

What is claimed is:

1. A composition, comprising at least one of:
(a) an isolated class I HLA trimolecular complex produced in vitro, the trimolecular complex comprising A*02 heavy chain, beta-2-microglobulin, and a peptide comprising SEQ ID NO:316;
(b) an isolated class I HLA trimolecular complex produced in vitro, the trimolecular complex comprising A*02 heavy chain, beta-2-microglobulin, and a peptide comprising SEQ ID NO:317;
(c) an isolated class I HLA trimolecular complex produced in vitro, the trimolecular complex comprising A*02 heavy chain, beta-2-microglobulin, and a peptide comprising SEQ ID NO:318;
(d) an isolated class I HLA trimolecular complex produced in vitro, the trimolecular complex comprising A*02 heavy chain, beta-2-microglobulin, and a peptide comprising SEQ ID NO:319; and
(e) an isolated class I HLA trimolecular complex produced in vitro, the trimolecular complex comprising A*02 heavy chain, beta-2-microglobulin, and a peptide comprising SEQ ID NO:320.

2. The composition of claim 1, further defined as comprising at least two of (a)-(e).

3. The composition of claim 1, further defined as comprising at least three of (a)-(e).

4. The composition of claim 1, further defined as comprising at least four of (a)-(e).

5. The composition of claim 1, further defined as comprising (a)-(e).

6. The composition of claim 1, wherein the A*02 heavy chain of at least one of (a)-(e) is further defined as a soluble, recombinantly produced A*02 heavy chain.

7. The composition of claim 1, wherein the peptide of at least one of (a)-(e) is a synthetic peptide.

8. The composition of claim 1, wherein the peptide of at least one of (a)-(e) is further defined as a peptide having a length of from 9 to 13 amino acids.

9. The composition of claim 1, wherein the peptides of (a)-(e) are further defined as:
(a) a peptide consisting essentially of a fragment of SEQ ID NO:327 and comprising SEQ ID NO:316;
(b) a peptide consisting essentially of a fragment of SEQ ID NO:328 and comprising SEQ ID NO:317;
(c) a peptide consisting essentially of a fragment of SEQ ID NO:329 and comprising SEQ ID NO:318;
(d) a peptide consisting essentially of a fragment of SEQ ID NO:330 and comprising SEQ ID NO:319; and
(e) a peptide consisting essentially of a fragment of SEQ ID NO:331 and comprising SEQ ID NO:320.

10. A composition, comprising at least one of:
(a) an isolated class I HLA trimolecular complex produced in vitro, the trimolecular complex comprising A*02 heavy chain, beta-2-microglobulin, and a peptide consisting essentially of a fragment of SEQ ID NO:327 and comprising SEQ ID NO:316;
(b) an isolated class I HLA trimolecular complex produced in vitro, the trimolecular complex comprising A*02 heavy chain, beta-2-microglobulin, and a peptide consisting essentially of a fragment of SEQ ID NO:328 and comprising SEQ ID NO:317;
(c) an isolated class I HLA trimolecular complex produced in vitro, the trimolecular complex comprising A*02 heavy chain, beta-2-microglobulin, and a peptide consisting essentially of a fragment of SEQ ID NO:329 and comprising SEQ ID NO:318;
(d) an isolated class I HLA trimolecular complex produced in vitro, the trimolecular complex comprising A*02 heavy chain, beta-2-microglobulin, and a peptide consisting essentially of a fragment of SEQ ID NO:330 and comprising SEQ ID NO:319; and
(e) an isolated class I HLA trimolecular complex produced in vitro, the trimolecular complex comprising A*02 heavy chain, beta-2-microglobulin, and a peptide consisting essentially of a fragment of SEQ ID NO:331 and comprising SEQ ID NO:320.

11. The composition of claim 10, further defined as comprising at least two of (a)-(e).

12. The composition of claim 10, further defined as comprising at least three of (a)-(e).

13. The composition of claim 10, further defined as comprising at least four of (a)-(e).

14. The composition of claim 10, further defined as comprising (a)-(e).

15. The composition of claim 10, wherein the A*02 heavy chain of at least one of (a)-(e) is further defined as a soluble, recombinantly produced A*02 heavy chain.

16. The composition of claim 10, wherein the peptide of at least one of (a)-(e) is a synthetic peptide.

17. The composition of claim 10, wherein the peptide of at least one of (a)-(e) is further defined as a peptide having a length of from 9 to 13 amino acids.

18. A composition comprising an isolated class I HLA trimolecular complex produced in vitro, the trimolecular complex comprising A*02 heavy chain, beta-2-microglobulin, and a peptide consisting essentially of a fragment of SEQ ID NO:330 and comprising SEQ ID NO:319.

19. The composition of claim 18, wherein the A*02 heavy chain of at least one of (a)-(e) is further defined as a soluble, recombinantly produced A*02 heavy chain.

20. The composition of claim 18, wherein the peptide of at least one of (a)-(e) is a synthetic peptide.

21. An isolated composition, comprising:
class I HLA A*02 heavy chain or DNA encoding A*02 heavy chain;
beta-2-microglobulin or DNA encoding beta-2-microglobulin; and
at least one peptide comprising one of SEQ ID NOS:316-320, or DNA encoding the at least one peptide.


SEQUENCE LISTING (339 sequences)

SEQ ID NO: 1 - protein, 10 aa, Homo sapiens
EQMFEDIISL

SEQ ID NO: 2 - protein, 9 aa, Homo sapiens
IPCLLISFL

SEQ ID NO: 3 - protein, 10 aa, Homo sapiens
STTAICATGL

SEQ ID NO: 4 - protein, 8 aa, Homo sapiens
APAQNPEL

SEQ ID NO: 5 - protein, 9 aa, Homo sapiens
LVMAPRTVL

SEQ ID NO: 6 - protein, 9 aa, Homo sapiens
  MISC_FEATURE (5)..(5): N or S
APFIXPADX

SEQ ID NO: 7 - protein, 9 aa, Homo sapiens
TPQSNRPVM

SEQ ID NO: 8 - protein, 9 aa, Homo sapiens
AARPATSTL

SEQ ID NO: 9 - protein, 9 aa, Homo sapiens
MAMMAALMA

SEQ ID NO: 10 - protein, 9 aa, Homo sapiens
IATVDSYVI

SEQ ID NO: 11 - protein, 11 aa, Homo sapiens
SPNQARAQAAL

SEQ ID NO: 12 - protein, 10 aa, Homo sapiens
GPRTAALGLL

SEQ ID NO: 13 - protein, 10 aa, Homo sapiens
NPNQNKNVAL

SEQ ID NO: 14 - protein, 9 aa, Homo sapiens
RPYSNVSNL

SEQ ID NO: 15 - protein, 9 aa, Homo sapiens
LPQANRDTL

SEQ ID NO: 16 - protein, 9 aa, Homo sapiens
QPRYPVNSV

SEQ ID NO: 17 - protein, 8 aa, Homo sapiens
APAYSRAL

SEQ ID NO: 18 - protein, 9 aa, Homo sapiens
APKRPPSAF

SEQ ID NO: 19 - protein, 11 aa, Homo sapiens
AASKERSGVSL

SEQ ID NO: 20 - protein, 9 aa, Homo sapiens
FIISRTQAL

SEQ ID NO: 21 - protein, 9 aa, Homo sapiens
SLAGSLRSV

SEQ ID NO: 22 - protein, 8 aa, Homo sapiens
YGMPRQIL

SEQ ID NO: 23 - protein, 8 aa, Homo sapiens
MIIINKFV

SEQ ID NO: 24 - protein, 12 aa, Homo sapiens
ALWDIETGQQTV

SEQ ID NO: 25 - protein, 9 aa, Homo sapiens
VLMTEDIKL

SEQ ID NO: 26 - protein, 10 aa, Homo sapiens
YIYDKDMEII

SEQ ID NO: 27 - protein, 9 aa, Homo sapiens
ALMPVLNQV

SEQ ID NO: 28 - protein, 9 aa, Homo sapiens
DLIIKGISV

SEQ ID NO: 29 - protein, 9 aa, Homo sapiens
QLVDIIEKV

SEQ ID NO: 30 - protein, 9 aa, Homo sapiens
IMLEALERV

SEQ ID NO: 31 - protein, 8 aa, Homo sapiens
DAYIRIVL

SEQ ID NO: 32 - protein, 9 aa, Homo sapiens
ILDPHVVLL

SEQ ID NO: 33 - protein, 9 aa, Homo sapiens
DAKIRIFDL

SEQ ID NO: 34 - protein, 9 aa, Homo sapiens
ALLDKLYAL

SEQ ID NO: 35 - protein, 10 aa, Homo sapiens
FMFDEKLVTV

SEQ ID NO: 36 - protein, 9 aa, Homo sapiens
SLAQYLINV

SEQ ID NO: 37 - protein, 9 aa, Homo sapiens
SLLQTLYKV

SEQ ID NO: 38 - protein, 9 aa, Homo sapiens
YMAELIERL

SEQ ID NO: 39 - protein, 9 aa, Homo sapiens
FLYLIIISY

SEQ ID NO: 40 - protein, 9 aa, Homo sapiens
SLLENLEKI

SEQ ID NO: 41 - protein, 9 aa, Homo sapiens
FLFNKVVNL

SEQ ID NO: 42 - protein, 9 aa, Homo sapiens
VLWDRTFSL

SEQ ID NO: 43 - protein, 9 aa, Homo sapiens
SLASVFVRL

SEQ ID NO: 44 - protein, 9 aa, Homo sapiens
FLMDFIHQV

SEQ ID NO: 45 - protein, 10 aa, Homo sapiens
FLWDEGFHQL

SEQ ID NO: 46 - protein, 9 aa, Homo sapiens
TALPRIFSL

SEQ ID NO: 47 - protein, 10 aa, Homo sapiens
KLWEMDNMLI

SEQ ID NO: 48 - protein, 9 aa, Homo sapiens
MVDGTLLLL

SEQ ID NO: 49 - protein, 9 aa, Homo sapiens
SLASLHPSV

SEQ ID NO: 50 - protein, 9 aa, Homo sapiens
YLLPAIVHI

SEQ ID NO: 51 - protein, 9 aa, Homo sapiens
SLASLHPSV

SEQ ID NO: 52 - protein, 10 aa, Homo sapiens
KLWDIINVNI

SEQ ID NO: 53 - protein, 9 aa, Homo sapiens
KYPENFFLL

SEQ ID NO: 54 - protein, 13 aa, Homo sapiens
YLLIEEDIRDLAA

SEQ ID NO: 55 - protein, 9 aa, Homo sapiens
DELQQPLEL

SEQ ID NO: 56 - protein, 9 aa, Homo sapiens
DEYEKLQVL

SEQ ID NO: 57 - protein, 9 aa, Homo sapiens
EEYQSLIRY

SEQ ID NO: 58 - protein, 9 aa, Homo sapiens
DDWKVIANY

SEQ ID NO: 59 - protein, 8 aa, Homo sapiens
DELLNKFV

SEQ ID NO: 60 - protein, 8 aa, Homo sapiens
DEFKVVVV

SEQ ID NO: 61 - protein, 8 aa, Homo sapiens
LEGLTVVY

SEQ ID NO: 62 - protein, 9 aa, Homo sapiens
VEEILSVAY

SEQ ID NO: 63 - protein, 9 aa, Homo sapiens
DEDVLRYQF

SEQ ID NO: 64 - protein, 9 aa, Homo sapiens
DEGTAFLVY

SEQ ID NO: 65 - protein, 8 aa, Homo sapiens
MEQVIFKY

SEQ ID NO: 66 - protein, 9 aa, Homo sapiens
NEQAFEEVF

SEQ ID NO: 67 - protein, 8 aa, Homo sapiens
VEEYVYEF

SEQ ID NO: 68 - protein, 8 aa, Homo sapiens
DEIQVPVL

SEQ ID NO: 69 - protein, 9 aa, Homo sapiens
DEYQFVERL

SEQ ID NO: 70 - protein, 10 aa, Homo sapiens
DEYSIFPQTY

SEQ ID NO: 71 - protein, 9 aa, Homo sapiens
DEYSLVREL

SEQ ID NO: 72 - protein, 8 aa, Homo sapiens
EEVETFAF

SEQ ID NO: 73 - protein, 9 aa, Homo sapiens
NENDIRVMF

SEQ ID NO: 74 - protein, 9 aa, Homo sapiens
DEYDFYRSF

SEQ ID NO: 75 - protein, 10 aa, Homo sapiens
DEFQLLQAQY

SEQ ID NO: 76 - protein, 9 aa, Homo sapiens
DEFEFLEKA

SEQ ID NO: 77 - protein, 8 aa, Homo sapiens
DEMKVLVL

SEQ ID NO: 78 - protein, 9 aa, Homo sapiens
DERVFVALY

SEQ ID NO: 79 - protein, 9 aa, Homo sapiens
IENPFGETF

SEQ ID NO: 80 - protein, 9 aa, Homo sapiens
SEFELLRSY

SEQ ID NO: 81 - protein, 9 aa, Homo sapiens
DEGRLVLEF

SEQ ID NO: 82 - protein, 8 aa, Homo sapiens
DEGWFLIL

SEQ ID NO: 83 - protein, 8 aa, Homo sapiens
DEISFVNF

SEQ ID NO: 84 - protein, 8 aa, Homo sapiens
SEVLSWQF

SEQ ID NO: 85 - protein, 11 aa, Homo sapiens
YEILLGKATLY

SEQ ID NO: 86 - protein, 9 aa, Homo sapiens
YENLLAVAF

SEQ ID NO: 87 - protein, 9 aa, Homo sapiens
DETQIFSYF

SEQ ID NO: 88 - protein, 9 aa, Homo sapiens
MEPLRVLEL

SEQ ID NO: 89 - protein, 9 aa, Homo sapiens
MPLGKTLPC

SEQ ID NO: 90 - protein, 9 aa, Homo sapiens
VYMDWYEKF

SEQ ID NO: 91 - protein, 8 aa, Homo sapiens
SELLIHVF

SEQ ID NO: 92 - protein, 8 aa, Homo sapiens
DEHLITFF

SEQ ID NO: 93 - protein, 9 aa, Homo sapiens
DEFKIGELF

SEQ ID NO: 94 - protein, 11 aa, Homo sapiens
DELEIIEGMKF

SEQ ID NO: 95 - protein, 10 aa, Homo sapiens
KYLLSATKLR

SEQ ID NO: 96 - protein, 9 aa, Homo sapiens
SEIELFRVF

SEQ ID NO: 97 - protein, 9 aa, Homo sapiens
LEDVLPLAF

SEQ ID NO: 98 - protein, 7 aa, Homo sapiens
GSHSMRY

SEQ ID NO: 99 - protein, 7 aa, Homo sapiens
NDHFVKL

SEQ ID NO: 100 - protein, 10 aa, Homo sapiens
GLMTTVHAIT

SEQ ID NO: 101 - protein, 9 aa, Homo sapiens
ALNDHFVKL

SEQ ID NO: 102 - protein, 9 aa, Homo sapiens
RLTPKLMEV

SEQ ID NO: 103 - protein, 9 aa, Homo sapiens
KLEEIIHQI

SEQ ID NO: 104 - protein, 12 aa, Homo sapiens
KLLEGEESRISL

SEQ ID NO: 105 - protein, 9 aa, Homo sapiens
ALNEKLVNL

SEQ ID NO: 106 - protein, 9 aa, Homo sapiens
LLDVPTAAV

SEQ ID NO: 107 - protein, 9 aa, Homo sapiens
AVGKVIPEL

SEQ ID NO: 108 - protein, 11 aa, Homo sapiens
GLMTTVHAITA

SEQ ID NO: 109 - protein, 11 aa, Homo sapiens
TLAEVERLKGL

SEQ ID NO: 110 - protein, 13 aa, Homo sapiens
GLMTTVHAITATQ

SEQ ID NO: 111 - protein, 9 aa, Homo sapiens
GVLDNIQAV

SEQ ID NO: 112 - protein, 9 aa, Homo sapiens
ALDKATVLL

SEQ ID NO: 113 - protein, 9 aa, Homo sapiens
KVPEWVDTV

SEQ ID NO: 114 - protein, 9 aa, Homo sapiens
KMLEKLPEL

SEQ ID NO: 115 - protein, 8 aa, Homo sapiens
FLGRINEI

SEQ ID NO: 116 - protein, 9 aa, Homo sapiens
GLIEKNIEL

SEQ ID NO: 117 - protein, 10 aa, Homo sapiens
KVFDPVPVGV

SEQ ID NO: 118 - protein, 12 aa, Homo sapiens
GLMTTVHAITAT

SEQ ID NO: 119 - protein, 9 aa, Homo sapiens
FAITAIKGV

SEQ ID NO: 120 - protein, 9 aa, Homo sapiens
SMTLAIHEI

SEQ ID NO: 121 - protein, 10 aa, Homo sapiens
LLDANLNIKI

SEQ ID NO: 122 - protein, 10 aa, Homo sapiens
TLWDIQKDLK

SEQ ID NO: 123 - protein, 10 aa, Homo sapiens
KMYEEFLSKV

SEQ ID NO: 124 - protein, 13 aa, Homo sapiens
FLASESLIKQIPR

SEQ ID NO: 125 - protein, 13 aa, Homo sapiens
KLFDDDETGKISF

SEQ ID NO: 126 - protein, 9 aa, Homo sapiens
SLDQPTQTV

SEQ ID NO: 127 - protein, 9 aa, Homo sapiens
GIDSSSPEV

SEQ ID NO: 128 - protein, 9 aa, Homo sapiens
KAPPAPLAA

SEQ ID NO: 129 - protein, 9 aa, Homo sapiens
ILDKKVEKV

SEQ ID NO: 130 - protein, 8 aa, Homo sapiens
KLDEGNSL

SEQ ID NO: 131 - protein, 9 aa, Homo sapiens
VVQDGIVKA

SEQ ID NO: 132 - protein, 9 aa, Homo sapiens
VVQDGIVKA

SEQ ID NO: 133 - protein, 9 aa, Homo sapiens
YLEAGGTKV

SEQ ID NO: 134 - protein, 9 aa, Homo sapiens
ALSDGVHKI

SEQ ID NO: 135 - protein, 9 aa, Homo sapiens
GLAEDSPKM

SEQ ID NO: 136 - protein, 9 aa, Homo sapiens
EAAHVAEQL

SEQ ID NO: 137 - protein, 9 aa, Homo sapiens
AQAPDLQRV

SEQ ID NO: 138 - protein, 9 aa, Homo sapiens
GVYGDVHRV

SEQ ID NO: 139 - protein, 9 aa, Homo sapiens
YLTHDSPSV

SEQ ID NO: 140 - protein, 9 aa, Homo sapiens
RLDDVSNDV

SEQ ID NO: 141 - protein, 11 aa, Homo sapiens
KLMELHGEGSS

SEQ ID NO: 142 - protein, 11 aa, Homo sapiens
KMWDPHNDPNA

SEQ ID NO: 143 - protein, 9 aa, Homo sapiens
ALSDGVHKI

SEQ ID NO: 144 - protein, 9 aa, Homo sapiens
KLDPTKTTL

SEQ ID NO: 145 - protein, 9 aa, Homo sapiens
RVPPPPPIA

SEQ ID NO: 146 - protein, 10 aa, Homo sapiens
FIQTQQLHAA

SEQ ID NO: 147 - protein, 9 aa, Homo sapiens
SLTGHISTV

SEQ ID NO: 148 - protein, 9 aa, Homo sapiens
KIAPNTPQL

SEQ ID NO: 149 - protein, 9 aa, Homo sapiens
NLDPAVHEV

SEQ ID NO: 150 - protein, 9 aa, Homo sapiens
NMVAKVDEV

SEQ ID NO: 151 - protein, 9 aa, Homo sapiens
YLEDSGHTL

SEQ ID NO: 152 - protein, 9 aa, Homo sapiens
TLDEYTTRV

SEQ ID NO: 153 - protein, 9 aa, Homo sapiens
TLYEHNNEL

SEQ ID NO: 154 - protein, 9 aa, Homo sapiens
GLATDVQTV

SEQ ID NO: 155 - protein, 9 aa, Homo sapiens
QLLGSAHEV

SEQ ID NO: 156 - protein, 9 aa, Homo sapiens
GLDKQIQEL

SEQ ID NO: 157 - protein, 10 aa, Homo sapiens
YAYDGKDYIA

SEQ ID NO: 158 - protein, 9 aa, Homo sapiens
AVSDGVIKV

SEQ ID NO: 159 - protein, 9 aa, Homo sapiens
VLEDPVHAV

SEQ ID NO: 160 - protein, 9 aa, Homo sapiens
VMDSKIVQV

SEQ ID NO: 161 - protein, 9 aa, Homo sapiens
ILGYTEHQV

SEQ ID NO: 162 - protein, 9 aa, Homo sapiens
SMMDVDHQI

SEQ ID NO: 163 - protein, 9 aa, Homo sapiens
YAYDGKDYI

SEQ ID NO: 164 - protein, 11 aa, Homo sapiens
LMTTVHAITAT

SEQ ID NO: 165 - protein, 9 aa, Homo sapiens
AIVDKVPSV

SEQ ID NO: 166 - protein, 9 aa, Homo sapiens
SLAKIYTEA

SEQ ID NO: 167 - protein, 9 aa, Homo sapiens
SMLEDVQRA

SEQ ID NO: 168 - protein, 11 aa, Homo sapiens
VLLSDSNLHDA

SEQ ID NO: 169 - protein, 9 aa, Homo sapiens
YLDKVRALE

SEQ ID NO: 170 - protein, 8 aa, Homo sapiens
LLDVVHPA

SEQ ID NO: 171 - protein, 9 aa, Homo sapiens
LLDVVHPAA

SEQ ID NO: 172 - protein, 9 aa, Homo sapiens
ALASHLIEA

SEQ ID NO: 173 - protein, 9 aa, Homo sapiens
ALMDEVVKA

SEQ ID NO: 174 - protein, 9 aa, Homo sapiens
ILSGVVTKM

SEQ ID NO: 175 - protein, 9 aa, Homo sapiens
ILMEHIHKL

SEQ ID NO: 176 - protein, 9 aa, Homo sapiens
YMEEIYHRI

SEQ ID NO: 177 - protein, 9 aa, Homo sapiens
FLLEKGYEV

SEQ ID NO: 178 - protein, 10 aa, Homo sapiens
TLLEDGTFKV

SEQ ID NO: 179 - protein, 8 aa, Homo sapiens
GLGPTFKL

SEQ ID NO: 180 - protein, 9 aa, Homo sapiens
GLIDGRLTI

SEQ ID NO: 181 - protein, 9 aa, Homo sapiens
ALDEKLLNI

SEQ ID NO: 182 - protein, 9 aa, Homo sapiens
VLMTEDIKL

SEQ ID NO: 183 - protein, 9 aa, Homo sapiens
SLYEMVSRV

SEQ ID NO: 184 - protein, 10 aa, Homo sapiens
TLAEIAKVEL

SEQ ID NO: 185 - protein, 10 aa, Homo sapiens
GLDIDGIYRV

SEQ ID NO: 186 - protein, 12 aa, Homo sapiens
LLLDVPTAAVQA

SEQ ID NO: 187 - protein, 9 aa, Homo sapiens
AIIGGTFTV

SEQ ID NO: 188 - protein, 9 aa, Homo sapiens
GMASVISRL

SEQ ID NO: 189 - protein, 8 aa, Homo sapiens
TIAQLHAV

SEQ ID NO: 190 - protein, 9 aa, Homo sapiens
RLWPKIQGL

SEQ ID NO: 191 - protein, 10 aa, Homo sapiens
ALQELLSKGL

SEQ ID NO: 192 - protein, 9 aa, Homo sapiens
TLWGIQKEL

SEQ ID NO: 193 - protein, 9 aa, Homo sapiens
TLWPEVQKL

SEQ ID NO: 194 - protein, 9 aa, Homo sapiens
FLFNTENKL

SEQ ID NO: 195 - protein, 9 aa, Homo sapiens
ALLSAVTRL

SEQ ID NO: 196 - protein, 9 aa, Homo sapiens
SLLEKSLGL

SEQ ID NO: 197 - protein, 9 aa, Homo sapiens
KIADFGWSV

SEQ ID NO: 198 - protein, 9 aa, Homo sapiens
KLQEFLQTL

SEQ ID NO: 199 - protein, 11 aa, Homo sapiens
ALWEAKEGGLL

SEQ ID NO: 200 - protein, 11 aa, Homo sapiens
KLIGDPNLEFV

SEQ ID NO: 201 - protein, 9 aa, Homo sapiens
GLIENDALL

SEQ ID NO: 202 - protein, 9 aa, Homo sapiens
GLAKLIADV

SEQ ID NO: 203 - protein, 9 aa, Homo sapiens
TLIGLSIKV

SEQ ID NO: 204 - protein, 10 aa, Homo sapiens
LLLDVPTAAV

SEQ ID NO: 205 - protein, 9 aa, Homo sapiens
IMLEALERV

SEQ ID NO: 206 - protein, 11 aa, Homo sapiens
TLIDLPGITKV

SEQ ID NO: 207 - protein, 11 aa, Homo sapiens
ALLAGSEYLKL

SEQ ID NO: 208 - protein, 11 aa, Homo sapiens
KIIDEDGLLNL

SEQ ID NO: 209 - protein, 11 aa, Homo sapiens
TLQEVFERATF

SEQ ID NO: 210 - protein, 9 aa, Homo sapiens
RLIDLGVGL

SEQ ID NO: 211 - protein, 10 aa, Homo sapiens
GIVEGLMTTV

SEQ ID NO: 212 - protein, 9 aa, Homo sapiens
SMPDFDLHL

SEQ ID NO: 213 - protein, 11 aa, Homo sapiens
VLFDVTGQVRL

SEQ ID NO: 214 - protein, 10 aa, Homo sapiens
FLAEEGFYKF

SEQ ID NO: 215 - protein, 9 aa, Homo sapiens
ALVSSLHLL

SEQ ID NO: 216 - protein, 9 aa, Homo sapiens
ALLDKLYAL

SEQ ID NO: 217 - protein, 9 aa, Homo sapiens
GMYVFLHAV

SEQ ID NO: 218 - protein, 9 aa, Homo sapiens
AMIELVERL

SEQ ID NO: 219 - protein, 10 aa, Homo sapiens
VINDVRDIFL

SEQ ID NO: 220 - protein, 10 aa, Homo sapiens
FMFDEKLVTV

SEQ ID NO: 221 - protein, 11 aa, Homo sapiens
GVAESIHLWEV

SEQ ID NO: 222 - protein, 9 aa, Homo sapiens
GMYIFLHTV

SEQ ID NO: 223 - protein, 10 aa, Homo sapiens
GLLDPSVFHV

SEQ ID NO: 224 - protein, 9 aa, Homo sapiens
GLWDKFSEL

SEQ ID NO: 225 - protein, 11 aa, Homo sapiens
KLLDFGSLSNL

SEQ ID NO: 226 - protein, 10 aa, Homo sapiens
RLYPWGVVEV

SEQ ID NO: 227 - protein, 10 aa, Homo sapiens
KLFPDTPLAL

SEQ ID NO: 228 - protein, 10 aa, Homo sapiens
GLQDFDLLRV

SEQ ID NO: 229 - protein, 10 aa, Homo sapiens
ILYDIPDIRL

SEQ ID NO: 230 - protein, 9 aa, Homo sapiens
LLDVTPLSL

SEQ ID NO: 231 - protein, 9 aa, Homo sapiens
TLAKYLMEL

SEQ ID NO: 232 - protein, 11 aa, Homo sapiens
ALVEIGPRFVL

SEQ ID NO: 233 - protein, 9 aa, Homo sapiens
GIWGFIKGV

SEQ ID NO: 234 - protein, 9 aa, Homo sapiens
ILCPMIFNL

SEQ ID NO: 235 - protein, 9 aa, Homo sapiens
FLPSYIIDV

SEQ ID NO: 236 - protein, 9 aa, Homo sapiens
NLAEDIMRL

SEQ ID NO: 237 - protein, 10 aa, Homo sapiens
YLDIKGLLDV

SEQ ID NO: 238 - protein, 10 aa, Homo sapiens
IIMLEALERV

SEQ ID NO: 239 - protein, 9 aa, Homo sapiens
SIIGRLLEV

SEQ ID NO: 240 - protein, 9 aa, Homo sapiens
SLLDIIEKV

SEQ ID NO: 241 - protein, 11 aa, Homo sapiens
KIFEMGPVFTL

SEQ ID NO: 242 - protein, 10 aa, Homo sapiens
GVIAEILRGV

SEQ ID NO: 243 - protein, 9 aa, Homo sapiens
SLWSIISKV

SEQ ID NO: 244 - protein, 9 aa, Homo sapiens
SLFEGTWYL

SEQ ID NO: 245 - protein, 7 aa, Homo sapiens
RPKANSA

SEQ ID NO: 246 - protein, 8 aa, Homo sapiens
APRPPPKM

SEQ ID NO: 247 - protein, 8 aa, Homo sapiens
KPQDYKKR

SEQ ID NO: 248 - protein, 9 aa, Homo sapiens
RPTGGVGAV

SEQ ID NO: 249 - protein, 7 aa, Homo sapiens
ARPATSL

SEQ ID NO: 250 - protein, 8 aa, Homo sapiens
NLGSPRPL

SEQ ID NO: 251 - protein, 9 aa, Homo sapiens
AARPATSTL

SEQ ID NO: 252 - protein, 8 aa, Homo sapiens
RPGLKNNL

SEQ ID NO: 253 - protein, 9 aa, Homo sapiens
SPGPPTRKL

SEQ ID NO: 254 - protein, 9 aa, Influenza A virus
IPSIQSRGL

SEQ ID NO: 255 - protein, 9 aa, Influenza A virus
LPFDRTTVM

SEQ ID NO: 256 - protein, 10 aa, Homo sapiens
GPPGTGKTAL

SEQ ID NO: 257 - protein, 10 aa, Homo sapiens
APRGTGIVSA

SEQ ID NO: 258 - protein, 9 aa, Homo sapiens
APAGRKVGL

SEQ ID NO: 259 - protein, 9 aa, Homo sapiens
APGAPPRTL

SEQ ID NO: 260 - protein, 9 aa, Homo sapiens
APPPPPKAL

SEQ ID NO: 261 - protein, 9 aa, Homo sapiens
LPSSGRSSL

SEQ ID NO: 262 - protein, 9 aa, Homo sapiens
LPKPPGRGV

SEQ ID NO: 263 - protein, 9 aa, Homo sapiens
NLPLSNLAI

SEQ ID NO: 264 - protein, 9 aa, Homo sapiens
EPRPPHGEL

SEQ ID NO: 265 - protein, 9 aa, Homo sapiens
APNRPPAAL

SEQ ID NO: 266 - protein, 9 aa, Homo sapiens
APKRPPSAF

SEQ ID NO: 267 - protein, 9 aa, Homo sapiens
SPPSKPTVL

SEQ ID NO: 268 - protein, 9 aa, Homo sapiens
APRPVAVAV

SEQ ID NO: 269 - protein, 9 aa, Homo sapiens
RPPPIGAEV

SEQ ID NO: 270 - protein, 10 aa, Homo sapiens
RPAGKGSITI

SEQ ID NO: 271 - protein, 11 aa, Homo sapiens
SPGIPNPGAPL

SEQ ID NO: 272 - protein, 9 aa, Homo sapiens
RPQGGQDIL

SEQ ID NO: 273 - protein, 11 aa, Homo sapiens
PKFEVIEKPQA

SEQ ID NO: 274 - protein, 7 aa, Homo sapiens
VFLKPWI

SEQ ID NO: 275 - protein, 9 aa, Homo sapiens
ITAPPSRVL

SEQ ID NO: 276 - protein, 8 aa, Homo sapiens
TPEQIFQN

SEQ ID NO: 277 - protein, 10 aa, Homo sapiens
LPRGSSPSVL

SEQ ID NO: 278 - protein, 9 aa, Homo sapiens
GPREAFRQL

SEQ ID NO: 279 - protein, 8 aa, Homo sapiens
KPVIKKTL

SEQ ID NO: 280 - protein, 9 aa, Homo sapiens
SPRSGLIRV

SEQ ID NO: 281 - protein, 10 aa, Homo sapiens
LLPGENINLL

SEQ ID NO: 282 - protein, 8 aa, Homo sapiens
HLNEKRRF

SEQ ID NO: 283 - protein, 9 aa, Homo sapiens
TQFVRFDSD

SEQ ID NO: 284 - protein, 9 aa, Homo sapiens
RVEPLRNEL

SEQ ID NO: 285 - protein, 9 aa, Homo sapiens
YQFTGIKKY

SEQ ID NO: 286 - protein, 9 aa, Homo sapiens
GPRSSLRVL

SEQ ID NO: 287 - protein, 7 aa, Homo sapiens
GPYPYTL

SEQ ID NO: 288 - protein, 8 aa, Homo sapiens
SPAKIHVF

SEQ ID NO: 289 - protein, 9 aa, Homo sapiens
DPMKARVVL

SEQ ID NO: 290 - protein, 9 aa, Homo sapiens
SPQEDKEVI

SEQ ID NO: 291 - protein, 9 aa, Homo sapiens
NPASKVIAL

SEQ ID NO: 292 - protein, 10 aa, Homo sapiens
RPSGKGIVEF

SEQ ID NO: 293 - protein, 8 aa, Homo sapiens
SPVPSRPL

SEQ ID NO: 294 - protein, 9 aa, Homo sapiens
APEEHPVLL

SEQ ID NO: 295 - protein, 7 aa, Homo sapiens
SPKIRRL

SEQ ID NO: 296 - protein, 9 aa, Homo sapiens
LVFQPVAEL

SEQ ID NO: 297 - protein, 9 aa, Homo sapiens
GPLDIEWLI

SEQ ID NO: 298 - protein, 9 aa, Homo sapiens
RIVPRFSEL

SEQ ID NO: 299 - protein, 9 aa, Homo sapiens
YPKRPLLGL

SEQ ID NO: 300 - protein, 10 aa, Homo sapiens
YPFKPPKVAF

SEQ ID NO: 301 - protein, 9 aa, Homo sapiens
APKIGPLGL

SEQ ID NO: 302 - protein, 9 aa, Homo sapiens
AVLDELKVA

SEQ ID NO: 303 - protein, 9 aa, Homo sapiens
NLMHISYEA

SEQ ID NO: 304 - protein, 8 aa, Homo sapiens
LLDVPTAA

SEQ ID NO: 305 - protein, 10 aa, Homo sapiens
FLKEPALNEA

SEQ ID NO: 306 - protein, 9 aa, Homo sapiens
SLDQSVTHL

SEQ ID NO: 307 - protein, 9 aa, Homo sapiens
KIVVVTAGV

SEQ ID NO: 308 - protein, 10 aa, Homo sapiens
HLIEQDFPGM

SEQ ID NO: 309 - protein, 10 aa, Homo sapiens
FGVEQDVDMV

SEQ ID NO: 310 - protein, 10 aa, West Nile virus
RLDDDGNFQL

SEQ ID NO: 311 - protein, 9 aa, West Nile virus
ATWAENIQV

SEQ ID NO: 312 - protein, 9 aa, West Nile virus
SVGGVFTSV

SEQ ID NO: 313 - protein, 9 aa, West Nile virus
YTMDGEYRL

SEQ ID NO: 314 - protein, 9 aa, West Nile virus
SLTSINVQA

SEQ ID NO: 315 - protein, 9 aa, West Nile virus
SLFGQRIEN

SEQ ID NO: 316 - protein, 9 aa, Homo sapiens
KIGEGTYGV

SEQ ID NO: 317 - protein, 9 aa, Homo sapiens
ILDQKINEV

SEQ ID NO: 318 - protein, 9 aa, Homo sapiens
GLNEEIARV

SEQ ID NO: 319 - protein, 9 aa, Homo sapiens
FLSELTQQL

SEQ ID NO: 320 - protein, 9 aa, Homo sapiens
ALMPVLNQV

SEQ ID NO: 321 - protein, 9 aa, Homo sapiens
KILDLETQL

SEQ ID NO: 322 - protein, 10 aa, Homo sapiens
AQYEHDLEVA

SEQ ID NO: 323 - protein, 9 aa, Homo sapiens
TLYEAVREV

SEQ ID NO: 324 - protein, 9 aa, Homo sapiens
SLLEKSLGL

SEQ ID NO: 325 - protein, 9 aa, Homo sapiens
SLFGGSVKL

SEQ ID NO: 326 - protein, 9 aa, Homo sapiens
SLFPGKLEV

SEQ ID NO: 327 - protein, 298 aa, Homo sapiens
MENFQKVEKIGEGTYGVVYKARNKLTGEVVALKKIRLDTETEGVPSTAIREISLLKELNHPNIVKLLDVIHTENKLYLVFEFLHQDLKKFMDASALTGIPLPLIKSYLFQLLQGLAFCHSHRVLHRDLKPQNLLINTEGAIKLADFGLARAFGVPVRTYTHEVVTLWYRAPEILLGCKYYSTAVDIWSLGCIFAEMVTRRALFPGDSEIDQLFRIFRTLGTPDEVVWPGVTSMPDYKPSFPKWARQDFSKVVPPLDEDGRSLLSQMLHYDPNKRISAKAALAHPFFQDVTKPVPHLRL

SEQ ID NO: 328 - protein, 461 aa, Homo sapiens
MNNFGNEEFDCHFLDEGFTAKDILDQKINEVSSSDDKDAFYVADLGDILKKHLRWLKALPRVTPFYAVKCNDSKAIVKTLAATGTGFDCASKTEIQLVQSLGVPPERIIYANPCKQVSQIKYAANNGVQMMTFDSEVELMKVARAHPKAKLVLRIATDDSKAVCRLSVKFGATLRTSRLLLERAKELNIDVVGVSFHVGSGCTDPETFVQAISDARCVFDMGAEVGFSMYLLDIGGGFPGSEDVKLKFEEITGVINPALDKYFPSDSGVRIIAEPGRYYVASAFTLAVNIIAKKIVLKEQTGSDDEDESSEQTFMYYVNDGVYGSFNCILYDHAHVKPLLQKRPKPDEKYYSSSIWGPTCDGLDRIVERCDLPEMHVGDWMLFENMGAYTVAAASTFNGFQRPTIYYVMSGPAWQLMQQFQNPDFPPEVEEQDASTLPVSCAWESGMKRHRAACASASINV

SEQ ID NO: 329 - protein, 524 aa, Homo sapiens
MKRSSVSSGGAGRLSMQELRSQDVNKQGLYTPQTKEKPTFGKLSINKPTSERKVSLFGKRTSGHGSRNSQLGIFSSSEKIKDPRPLNDKAFIQQCIRQLCEFLTENGYAHNVSMKSLQAPSVKDFLKIFTFLYGFLCPSYELPDTKFEEEVPRIFKDLGYPFALSKSSMYTVGAPHTWPHIVAALVWLIDCIKIHTAMKESSPLFDDGQPWGEETEDGIMHNKLFLDYTIKCYESFMSGADSFDEMNAELQSKLKDLFNVDAFKLESLEAKNRALNEQIARLEQEREKEPNRLESLRKLKASLQGDVQKYQAYMSNLESHSAILDQKLNGLNEEIARVELECETIKQENTRLQNIIDNQKYSVADIERINHERNELQQTINKLTKDLEAEQQKLWNEELKYARGKEAIETQLAEYHKLARKLKLIPKGAENSKGYDFEIKFNPEAGANCLVKYRAQVYVPLKELLNETEEEINKALNKKMGLEDTLEQLNAMITESKRSVRTLKEEVQKLDDLYQQKKKKKKKK

SEQ ID NO: 330 - protein, 115 aa, Homo sapiens [sequence omitted]

SEQ ID NO: 331 - protein, 272 aa, Homo sapiens
MPGDHRRIRGPEESQPPQLYAADEEEAPGTRDPTRLRPVYARAGLLSQAKGSAYLEAGGTKVLCAVSGPRQAEGGERGGGPAGAGGEAPAALRGRLLCDFRRAPFAGRRRRAPPGGCEERELALALQEALEPAVRLGRYPRAQLEVSALLLEDGGSALAAALTAAALALADAGVEMYDLVVGCGLSLAPGPAPTWLLDPTRLEEERAAAGLTVALMPVLNQVAGLLGSGEGGLTESWAEAVRLGLEGCQRLYPVLQQSLVRAARRRGAAAQP

SEQ ID NO: 332 - protein, 829 aa, Homo sapiens
MSASSSGGSPRFPSCGKNGVTSLTQKKVLRAPCGAPSVTVTKSHKRGMKGDTVNVRRSVRVKTKVPWMPPGKSSARPVGCKWENPPHCLEITPPSSEKLVSVMRLSDLSTEDDDSGHCKMNRYDKKIDSLMNAVGCLKSEVKMQKGERQMAKRFLEERKEELEEVAHELAETEHENTVLRHNIERMKEEKDFTILQKKHLQQEKECLMSKLVEAEMDGAAAAKQVMALKDTIGKLKTEKQMTCTDINTLTRQKELLLQKLSTFEETNRTLRDLLREQHCKEDSERLMEQQGALLKRLAEADSEKARLLLLLQDKDKEVEELLQEIQCEKAQAKTASELSKSMESMRGHLQAQLRSKEAENSRLCMQIKNLERSGNQHKAEVEAIMEQLKELKQKGDRDKESLKKAIRAQKERAEKSEEYAEQLHVQLADKDLYVAEALSTLESWRSRYNQVVKEKGDLELEIIVLNDRVTDLVNQQQTLEEKMREDRDSLVERLHRQTAEYSAFKLENERLKASFAPMEDKLNQAHLEVQQLKASVKNYEGMIDNYKSQVMKTRLEADEVAAQLERCDKENKILKDEMNKEIEAARRQFQSQLADLQQLPDILKITEAKLAECQDQLQGYERKNIDLTAIISDLRSRIEHQGDKLEMAREKHQASQKENKQLSLKVDELERKLEATSAQNIEFLQVIAKREEAIHQSQLRLEEKTRECGTLARQLESAIEDARRQVEQTKEHALSKERAAQNKILDLETQLSRTKTELSQLRRSRDDADRRYQSRLQDLKDRLEQSESTNRSMQNYVQFLKSSYANVFGDGPYSTFLTSSPIRSRSPPA

SEQ ID NO: 333 - protein, 216 aa, Homo sapiens
MAAQGEPQVQFKLVLVGDGGTGKTTFVKRHLTGEFEKKYVATLGVEVHPLVFHTNRGPIKFNVWDTAGQEKFGGLRDGYYIQAQCAIIMFDVTSRVTYKNVPNWHRDLVRVCENIPIVLCGNKVDIKDRKVKAKSIVFHRKKNLQYYDISAKSNYNFEKPFLWLARKLIGDPNLEFVAMPALAPPEVVMDPALAAQYEHDLEVAQTTALPDEDDDL

SEQ ID NO: 334 - protein, 217 aa, Homo sapiens
MSSKVSRDTLYEAVREVLHGNQRKRRKFLETVELQISLKNYDPQKDKRFSGTVRLKSTPRPKFSVCVLGDQQHCDEAKAVDIPHMDIEALKKLNKNKKLVKKLAKKYDAFLASESLIKQIPRILGPGLNKAGKFPSLLTHNENMVAKVDEVKSTIKFQMKKVLCLAVAVGHVKMTDDELVYNIHLAVNFLVSLLKKNWQNVRALYIKSTMGKPQRLY

SEQ ID NO: 335 - protein, 174 aa, Homo sapiens
  misc_feature (42)..(42): Xaa can be any naturally occurring amino acid
MAAAAELSLLEKSLGLSKGNKYSAQGERQIPVLQTNNGPSLXGLTTIAAHLVKQANKEYLLGSTAEEKAXVQQWLEYRVTQVDGHSSKNDIHTLLXDLNSYLEDKVYLTGYNFTLADILLYYGLHRFIVDLTVQEKEKYLNVSRWFCHIQHYPGIRQHLSSVVFIKNRLYTNSH

SEQ ID NO: 336 - protein, 873 aa, Homo sapiens
MATFISMQLKKTSEVDLAKPLVKFIQQTYPSGGEEQAQYCRAAEELSKLRRAAVGRPLDKHEGALETLLRYYDQICSIEPKFPFSENQICLTFTWKDAFDKGSLFGGSVKLALASLGYEKSCVLFNCAALASQIAAEQNLDNDEGLKIAAKHYQFASGAFLHIKETVLSALSREPTVDISPDTVGTLSLIMLAQAQEVFFLKATRDKMKDAIIAKLANQAADYFGDAFKQCQYKDTLPKYFYFQEVFPVLAAKHCIMQANAEYHQSILAKQQKKFGEEIARLQHAAELIKTVASRYDEYVNVKDFSDKINRALAAAKKDNDFIYHDRVPDLKDLDPIGKATLVKSTPVNVPISQKFTDLFEKMVPVSVQQSLAAYNQRKADLINRSIAQMREATTLANGVLASLNLPAAIEDVSGDTVPQSILTKSRSVIEQGGIQTVDQLIKELPELLQRNREILDESLRLLDEEEATDNDLRAKFKERWQRTPSNELYKPLRAEGTNFRTVLDKAVQADGQVKECYQSHRDTIVLLCKPEPELNAAIPSANPAKTMQGSEVVNVLKSLLSNLDEVKKEREGLENDLKSVNFDMTSKFLTALAQDGVINEEALSVTELDRVYGGLTTKVQESLKKQEGLLKNIQVSHQEFSKMKQSNNEANLREEVLKNLATAYDNFVELVANLKEGTKFYNELTEILVRFQNKCSDIVFARKTERDELLKDLQQSIAREPSAPSIPTPAYQSSPAGGHAPTPPTPAPRTMPPTKPQPPARPPPPVLPANRAPSATAPSPVGAGTAAPAPSQTPGSAPPPQAQGPPYPTYPGYPGYCQMPMPMGYNPYAYGQYNMPYPPVYHQSPGQAPYPGPQQPSYPFPQPPQQSYYPQQ

SEQ ID NO: 337 - protein, 1269 aa, Homo sapiens
MEATGVLPFVRGVDLSGNDFKGGYFPENVKAMTSLRWLKLNRTGLCYLPEELAALQKLEHLSVSHNNLTTLHGELSSLPSLRAIVARANSLKNSGVPDDIFKLDDLSVLDLSHNQLTECPRELENAKNMLVLNLSHNSIDTIPNQLFINLTDLLYLDLSENRLESLPPQMRRLVHLQTLVLNGNPLLHAQLRQLPAMTALQTLHLRSTQRTQSNLPTSLEGLSNLADVDLSCNDLTRVPECLYTLPSLRRLNLSSNQITELSLCIDQWVHVETLNLSRNQLTSLPSAICKLSKLKKLYLNSNKLDFDGLPSGIGKLTNLEEFMAANNNLELVPESLCRCPKLRKLVLNKNHLVTLPEAIHFLTEIEVLDVRENPNLVMPPKPADRAAEWYNIDFSLQNQLRLAGASPATVAAAAAAGSGPKDPMARKMRLRRRKDSAQDDQAKQVLKGMSDVAQEKNKKQEESADARAPSGKVRRWDQGLEKPRLDYSEFFTEDVGQLPGLTIWQIENFVPVLVEEAFHGKFYEADCYIVLKTFLDDSGSLNWEIYYWIGGEATLDKKACSAIHAVNLRNYLGAECRTVREEMGDESEEFLQVFDNDISYIEGGTASGFYTVEDTHYVTRMYRVYGKKNIKLEPVPLKGTSLDPRFVFLLDRGLDIYVWRGAQATLSSTTKARLFAEKINKNERKGKAEITLLVQGQELPEFWEALGGEPSEIKKHVPEDFWPPQPKLYKVGLGLGYLELPQINYKLSVEHKQRPKVELMPRMRLLQSLLDTRCVYILDCWSDVFIWLGRKSPRLVRAAALKLGQELCGMLHRPRHATVSRSLEGTEAQVFKAKFKNWDDVLTVDYTRNAEAVLQSPGLSGKVKRDAEKKDQMKADLTALFLPRQPPMSLAEAEQLMEEWNEDLDGMEGFVLEGKKFARLPEEEFGHFYTQDCYVFLCRYWVPVEYEEEEKKEDKEEKAEGKEGEEATAEAEEKQPEEDFQCIVYFWQGREASNMGWLTFTFSLQKKFESLFPGKLEVVRMTQQQENPKFLSHFKRKFIIHRGKRKAVQGAQQPSLYQIRTNGSALCTRCIQINTDSSLLNSEFCFILKVPFESEDNQGIVYAWVGRASDPDEAKLAEDILNTMFDTSYSKQVINEGEEPENFFWVGIGAQKPYDDDAEYMKHTRLFRCSNEKGYFAVTEKCSDFCQDDLADDDIMLLDNGQEVYMWVGTQTSQVEIKLSLKACQVYIQHMRSKEHERPRRLRLVRKGNEQHAFTRCFHAWSAFCKALA

SEQ ID NO: 338 - protein, 9 aa, Homo sapiens
GLIEKNIEL

SEQ ID NO: 339 - protein, 9 aa, Homo sapiens
LLQEVEHQL